(19) United States
(12) Patent Application Publication
(10) Pub. No.: US 2026/0085046 A1
(43) Pub. Date: Mar. 26, 2026

(54) Dual Function Compounds For NADPH Oxidase Inhibition And Histone Deacetylase Inhibition

(71) Applicant: TaiwanJ Pharmaceuticals Co., Ltd., Hsinchu County (TW)

(72) Inventors: On LEE, Hsinchu City (TW); Sheng-Chieh LIEN, Hsinchu County (TW); Chia-Chueng CHEN, Hsinchu County (TW)

(21) Appl. No.: 19/293,394

(22) Filed: Aug. 7, 2025

Related U.S. Application Data
(60) Provisional application No. 63/699,432, filed on Sep. 26, 2024.

(51) Int. Cl. C07D 215/22; A61K 47/55

(52) U.S. Cl. CPC C07D 215/22; A61K 47/55

(57) ABSTRACT

Disclosed are compounds of Formula (I) below and pharmaceutically acceptable salts thereof:

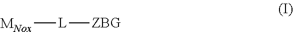

in which each of variables M_{Nox}, L and ZBG is defined herein. Also disclosed is a method for treating a cancer with a compound of Formula (I) or a pharmaceutically acceptable salt thereof and a pharmaceutical composition containing the same.

### CROSS-REFERENCE TO RELATED APPLICATIONS

This application claims the benefit of filing date of U.S. Provisional Application Ser. No. 63/699,432, filed Sep. 26, 2024 under 35 USC § 119(e)(1).

### BACKGROUND

### Field

The field of the disclosure relates compounds or pharmaceutically acceptable salts thereof which are inhibitors of the activity or function of the nicotinamide adenine dinucleotide phosphate (NADPH) oxidases (NOXs) or histone deacetylases (HDACs).

### Description of Related Art

Reactive oxygen species (ROS) are a group of short-lived intermediates produced by redox reactions or by electronic excitation of oxygen, such as free radicals (i.e., the superoxide anion and hydroxyl radical), as well as nonradical oxidant species (i.e., hydrogen peroxide, H₂O₂). The pivotal role of ROS in different biological processes, spanning from cell homeostasis to inhibition and activation of proteins together with gene transcription, is well established. Several cellular defense systems, such as enzymes that remove oxidants or oxidant scavengers, balance the formation and the reactions of these intermediates. Nevertheless, the increased production of oxidants, coupled with the failure of defense systems, causes an alteration of the proper equilibrium of the cellular redox state leading to the so-called “oxidative stress”. In this scenario, a cascade of several events lead to different human diseases including fibrosis, cancer, and cardiovascular and neurodegenerative disorders. Consequently, increasing attention has been paid to endogenous sources of ROS, including the mitochondrial respiratory chain, xanthine oxidase, lipoxygenases, and monoamine oxidase but mainly to nicotinamide adenine dinucleotide phosphate (NADPH) oxidases (NOXs). (Alessandra Cipriano et al., J. Med. Chem., 2023, 66, 11632-11655).

To date, seven membrane-crossing enzymes have been identified, namely, Nox1-5 and dual oxidases 1 and 2 (Duox1 and Duox2, respectively). The gp91^{phox} (renamed as Nox2) was the first identified isozyme, and it was in phagocytic cells. Subsequently, six additional homologues have been isolated in non-phagocytic cells (Nox1, Nox3, Nox4, Nox5, Duox1 and Duox2). (Sae Rom Lee et al., Biomol Ther, 2020, 28(1), 25-33).

Nox1-generated ROS play a vital role in the proliferation and invasions of colon cancer cells. An interesting study demonstrated the correlation between expressions of the Nox regulatory subunits and BRCA1 gene in ovarian cystadenocarcinoma, lung adenocarcinoma, and breast invasive carcinoma, suggesting that the high expression of regulatory subunits of Nox1 and Nox4 are related to the downregulation of BRCA1 gene expression, and these events may be associated with the progression of malignancy. In fact, the oxidative stress induced by Nox1 is responsible for the downregulation of the antiapoptotic protein surviving and for the subsequent initiation of ER+ breast tumor formation. Nox4 is the most frequently expressed Nox isoform in several malignancies such as neuroepithelial tumors, human melanomas, and lung, renal, colorectal, gastric, pancreatic, and ovarian cancers. (Alessandra Cipriano et al., J. Med. Chem., 2023, 66, 11632-11655).

Activation of protein kinase C (PKC) is involved in hyperglycemia-dependent nephropathy. High glucose (HG)-induced translocation of PKCα to renal membranes stimulates Nox2 activation. Nox2 activation by PKCa is the key downstream event of the advanced glycation end product (AGE)-receptor signaling in diabetic nephropathy (DN). HG, angiotensin II or TGFβ stimulate Nox4-derived ROS generation, resulting in fibrotic response in mesangial cells, podocytes and tubular cells. (Sae Rom Lee et al., Biomol Ther, 2020, 28(1), 25-33). Glomerular basement membrane thickening, mesangial expansion, overexpression of extracellular matrix (ECM) proteins, tubulointerstitial fibrosis, and glomerulosclerosis can contribute to the progression of DN. Deletion of Nox4 can attenuate mesangial hypertrophy and ECM accumulation in diabetic conditions. (Hye Eun Lee et al., Kidney Res Clin Pract, 2022, 41 (Suppl 2), S89-S98).

Lung fibrosis has diverse aetiologies including drugs, chemical insults, radiation, occupational exposures and connective tissue diseases; alternatively, it may be ‘idiopathic’ in nature. Damage-induced alveolar epithelial cell death causes the release of profibrotic mediators, including TGF-β, which activate resident lung fibroblasts and trigger their differentiation into myofibroblasts, resulting in heightened ECM synthetic capacity and resistance to apoptosis. Importantly, TGF-β induces Nox4-mediated ROS production in lung fibroblasts, which sustains myofibroblast differentiation and drives fibrosis progression in lung disease. (Victor J. Thannickal et al., J Cell Mol Med., 2023, 27, 471-481). The protein expression of Nox isoforms Nox1, Nox2, Nox4, and Nox5 was significantly upregulated in lung tissue sections of patients with end-stage COPD. In mice exposed to acute cigarette smoke (ACS) using a standard TE-10 smoking machine, the protein expression of Nox1, Nox2, and Nox4 in lung tissues was significantly upregulated. (Xinjing Wang et al., Antioxidants, 2022, 11, 1539).

A dual Nox1/4 inhibitor GKT137831 (brand name: Setanaxib), a pyrazolopyridine compound, was developed by Genkyotexn (Stockholm, Sweden). In October 2013, GKT137831 entered a clinical trial to evaluate its efficacy in oral administration in type 2 diabetes patients with maximal inhibition of the renin-angiotensin-aldosterone system and residual albuminuria. The study concluded in March 2015, but the results are still not available. Currently, GKT137831 is being evaluated in two different clinical trials in patients with primary biliary cholangitis (PBC) and liver stiffness, as well as in patients with idiopathic pulmonary fibrosis. (Alessandra Cipriano et al., J. Med. Chem., 2023, 66, 11632-11655). The Ewha-18278 compound was first developed for osteoporosis treatment by Joo et al. The compound was transferred to AptaBio Corp. (Suwon, Korea) and is now referred to as APX-115. (Hye Eun Lee et al., Kidney Res Clin Pract, 2022, 41 (Suppl 2), S89-S98). APX-115 is a first-in-class pan Nox inhibitor. APX-115 suppressed urinary albumin excretion and preserved creatinine clearance in db/db mice. In diabetic kidneys, APX-115 significantly suppressed mesangial expansion. Comparative study showed that APX-115 is more effective than dual Nox1/4 inhibitor GKT137831. (Sae Rom Lee et al., Biomol Ther, 2020, 28(1), 25-33). However, no Nox inhibitors have been approved so far.

Epigenetics refers to a reversible or inheritable process that regulates gene expression without altering DNA nucleotide sequence. There are several primary epigenetic mechanisms: DNA/RNA methylation, RNA editing, noncoding RNA-mediated regulation, histone variant, histone modification and chromatin remodeling. Histone modification, especially acetylation/deacetylation on histone mediated by histone deacetylases (HDACs) and histone acetyltransferases (HATs), is a major epigenetic mechanism. (Tao Liang et al., Acta Pharmaceutica Sinica B, 2023, 13(6), 2425-2463). Epigenetics can turn on/off gene expression and thus plays a crucial role in tumorigenesis and cancer progression. Abnormal epigenetic alterations and destroyed epigenetic integrity are common characteristics of tumor cells. Among all epigenetic mechanisms, histone modifications have been shown to be important in carcinogenesis. (Rihan Hai et al., Front Oncol., 2021, 11, 700947). HDACs deacetylate the ε-NH₂ group of lysine in nucleosome histone, which tightens the charged association of histone tails with DNA and prevents the binding of transcription co-factors to DNA, thereby repressing gene transcription. Conversely, HATs acetylate the ε-NH₂ group and relax the charged interaction, followed by greater accesses of transcription co-factors to DNA, thus promoting gene transcription. (Tao Liang et al., Acta Pharmaceutica Sinica B, 2023, 13(6), 2425-2463).

In addition to histones, HDAC can modulate the function of many other proteins involved in the regulation of cell survival and proliferation, angiogenesis, inflammation, and immunity. Moreover, since HDACs play an important role in the regulation of gene expression, it is no surprise that deregulated HDAC activity is associated with many different diseases, especially, cancers, inflammation, and depression and is thus an attractive drug target.

The human HDAC family counts 18 HDACs, which are further classified into two categories based on their catalytic mechanism: 11 of them are zinc-dependent metalloenzymes (classes I, II, IV) and the remaining 7 (class III, also named sirtuins) require nicotinamide adenine dinucleotide as a co-factor for their enzymatic activity. The four classes of HDACs are also divided according to their cellular localization, mechanism of catalysis, structure and expression pattern. Class I HDACs are located in the nucleus and consist of HDACs 1, 2, 3, and 8. Class II HDACs, which are localized in both the nucleus and cytoplasm, comprise HDACs 4, 5, 6, 7, 9, and 10, and are further subdivided into classes IIA (HDACs 4, 5, 7, and 9) and IIB (HDACs 6 and 10). Class III HDACs are found in the nucleus, cytoplasm and mitochondria. HDAC11 belongs to class IV and is present in the nucleus and cytoplasm. (Angelica Ferro et al., Med Res Rev., 2023, 43, 2177-2236).

Zinc ion-dependent HDACs possess a highly conserved and homologous catalytic domain: a catalytic channel, a zinc cation and some secondary pockets. To achieve good inhibition of HDACs, various HDAC inhibitors were designed according to the unique structure of the target enzymes. HDAC inhibitor generally possesses three common pharmacophore models: surface binding region (cap), linker and zinc binding group (ZBG), whereas there are also a few HDAC inhibitors with only two functional fragments of linker and ZBG. (Tao Liang et al., Acta Pharmaceutica Sinica B, 2023, 13(6), 2425-2463).

Cap group binds to the pockets in the rim of HDACs channel, which can increase the affinity of HDAC inhibitor for target enzyme and block the entry of substrate into HDAC catalytic channel. The linker is responsible for delivering ZBG to chelate zinc cation. A suitable linker is capable of helping HDAC inhibitor achieve good inhibitory activity. A short linker cannot allow ZBG to interact with zinc ion well, resulting in poor binding, while a long linker may affect the interaction of the cap group with the pocket in the rim of the catalytic channel and reduce the affinity of HDAC inhibitor for target enzyme. ZBG, another key group for HDAC inhibitor, can chelate zinc ion in the catalytic center and inhibit the catalytic activity of HDACs. (Tao Liang et al., Acta Pharmaceutica Sinica B, 2023, 13(6), 2425-2463). HDAC inhibitors can be divided in 4 main chemical classes, based on their zinc-binding group or cap group type: alkanoic acids, hydroxamic acids, depsipeptides (with thiol zinc-binding group), or macrocycles and ortho-aminoanilides. The ortho-aminoanilide family of HDAC inhibitor is also broadly referred to as the benzamide family; however, the term benzamide describes only a subcategory of the larger ortho-aminoanilide family. (Florence F. Wagner et al., Neurotherapeutics, 2013, 10(4), 589-604).

To date, decades of efforts have caused five approved HDAC inhibitors, including vorinostat, romidepsin, belinostat, panobinostat and chidamide. Currently traditional HDAC inhibitors, although effective in approved indications, display severe adverse effects, off-target toxicities and poor therapeutic effects on solid tumors, which hamper their clinical applications. Therefore, it is urgent to develop the next-generation novel HDAC inhibitor, including isozyme-specific HDAC inhibitor, combination therapy, multitarget agent and HDAC PROTAC. Isozyme-specific HDAC inhibitor, an alternative to pan-HDAC inhibitor, will turn into very valuable drugs since they can be used as probes for the functional investigation of individual HDAC isozymes or as effective anticancer agents with superior therapeutic efficacy and safety profile. However, it is challenging for the development of isozyme-specific HDAC inhibitor due to the highly conserved catalytic domain, lack of crystal structure and little known about the enzymatic activities, biological functions of several HDAC isoforms. Combination therapy and multitarget agent are able to well address the poor therapeutic efficacy on solid tumors and drug resistance via synergistic or additive effects, which make them the promising trends for solid tumor therapy, especially difficult-to-treat solid tumors. (Tao Liang et al., Acta Pharmaceutica Sinica B, 2023, 13(6), 2425-2463).

In studies of many different types of tumors and other diseases, the combination of proteasome inhibitors and histone deacetylase inhibitors has shown strong synergistic effect. Although combination drugs can effectively overcome the problems of insensitivity and drug resistance to single-target drugs caused by tumor cell heterogeneity and adaptability, there are also problems such as differences in pharmacokinetic properties between different drugs, adverse drug-drug interactions, and poor patient compliance. The development of multi-target anti-cancer drugs is expected to overcome the above-mentioned shortcomings of drug combination while solving the problem of tumor insensitivity and drug resistance to single-target drugs.

### SUMMARY

The present disclosure relates to a compound or a pharmaceutically acceptable salt thereof as an inhibitor of NOXs or HDACs.

An aspect of this disclosure is drawn to the compound of the following Formula (I) or a pharmaceutically acceptable salt thereof: M_{Nox}-L-ZBG (I).

In this formula, ZBG is a hydroxamic acid group, an ortho-aminoanilide group or a derivative thereof;
M_{Nox} is or comprises a binding moiety that binds to Nox, and has a formula derived from a compound having the following Formula (II):

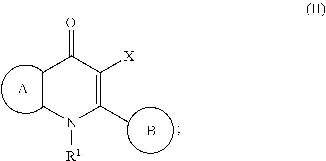

and
L is a bond or a linker covalently coupling M_{Nox} and ZBG, and is covalently linked to any location on the M_{Nox},
wherein,
A is an aminobenzene moiety, an ortho-dimethoxybenzene moiety, or a benzene moiety;
B is a phenyl group substituted with one or more nitro, methyl, CF₃, halogen, pyrrolidine, OR², NR³R⁴, or SO₂R³;
R¹ is H or C₁-C₁₀ alkyl;
R² is methyl, ethyl, n-propyl, or CF₃;
R³ is methyl, ethyl, n-propyl, or CF₃;
R⁴ is H; and
X is OH or NH₂.

Another aspect of this disclosure is a pharmaceutical composition for treating a cancer.

The pharmaceutical composition contains one of the compounds of Formula (I) described above or its pharmaceutically acceptable salt and at least one pharmaceutically acceptable carrier.

This disclosure also covers use of such a composition for the manufacture of a medicament for treating a cancer.

Still within the scope of the present disclosure is a method of treating a cancer.

The method includes administering to a subject in need thereof an effective amount of a compound of formula (I) or a pharmaceutically acceptable salt thereof.

Other novel features of the disclosure will become more apparent from the following detailed description when taken in conjunction with the accompanying drawings.

### DETAILED DESCRIPTION

One embodiment of the present disclosure is the compound of Formula (I) or a pharmaceutically acceptable salt thereof,

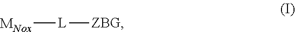

in which each of variables each of variables M_{Nox}, L and ZBG is defined as in the SUMMARY section.

In some embodiments of the present disclosure, Nox is selected from Nox1, Nox2, and Nox4. In some embodiments of the present disclosure, inhibitors of Nox that bind and/or inhibit Nox are disclosed in the prior art and include, but are not limited to compounds disclosed in U.S. Publication No. 2021/0276957, the content of which is incorporated herein by reference in its entirety.

Another embodiment of the present disclosure is the compound of the aforesaid embodiment, wherein M_{Nox} is derived from a compound of the following Formula (IIa):

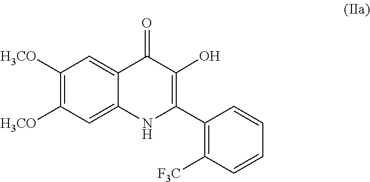

wherein L is covalently linked to any location on the M_{Nox}, or a pharmaceutically acceptable salt thereof.

Another embodiment of the present disclosure is the compound of the aforesaid embodiment, wherein M_{Nox} is derived from a compound of the following Formula (IIb):

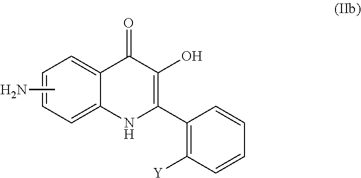

wherein Y is halogen, methyl, CF₃, OCH₃, OCH₂CH₃, OCH₂CH₂CH₃ or OCF₃, and L is covalently linked to any location on the M_{Nox}, or a pharmaceutically acceptable salt thereof. In some embodiments, Y is CF₃, F, CH₃, or OCH₃.

Another embodiment of the present disclosure is the compound of the aforesaid embodiment, wherein M_{Nox} is derived from a compound of the following Formula (IIc):

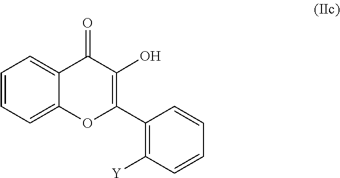

wherein Y is halogen, methyl, CF₃, OCH₃, OCH₂CH₃, OCH₂CH₂CH₃ or OCF₃, and L is covalently linked to any location on the M_{Nox},
or a pharmaceutically acceptable salt thereof. In some embodiments, Y is CF₃, F, CH₃, or OCH₃.

Another embodiment of the present disclosure is the compound of any one of the aforesaid embodiments, wherein ZBG is

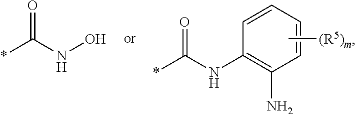

wherein each R⁵ is independently hydrogen or halogen; and m is 1, 2, 3 or 4.

Another embodiment of the present disclosure is the compound of any one of the aforesaid embodiments, wherein ZBG is

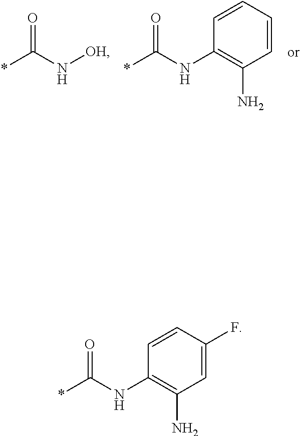

Another embodiment of the present disclosure is the compound of any one of the aforesaid embodiments, wherein L is selected from the group consisting of: *—(CH₂)ₙ—*, *—CH₂C(O)NH(CH₂)ₙ—*, *—C(O)—(CH₂)ₙ—*, *—NH—C(O)—(CH₂)ₙ—*, *—CH═CH—*,

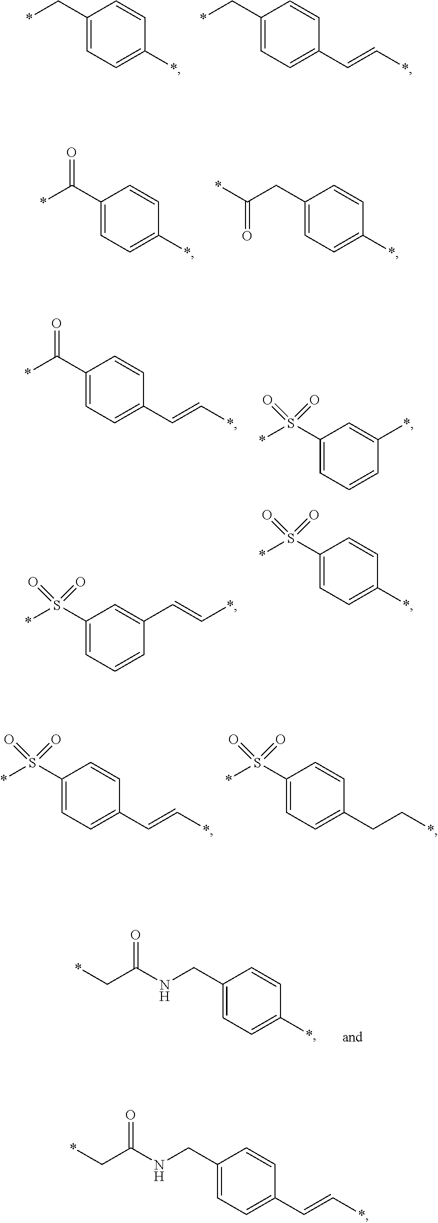

wherein n is 1, 2, 3, 4, 5, 6, 7, 8, 9, or 10.

Another embodiment of the present disclosure is the compound of any one of the aforesaid embodiments, wherein the compound has the following Formula (Ia):

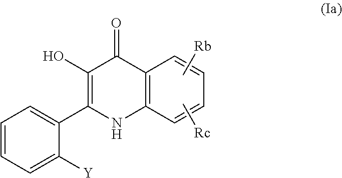

wherein one of Rb and Rc is *—O-L-ZBG, *—NH-L-ZBG, or *-L-ZBG; the other of Rb and Rc is H or OCH₃; and Y is halogen, methyl, CF₃, OCH₃, OCH₂CH₃, OCH₂CH₂CH₃ or OCF₃, or a pharmaceutically acceptable salt thereof. In some embodiments, Y is CF₃, F, CH₃, or OCH₃.

Another embodiment of the present disclosure is the compound of any one of the aforesaid embodiments, wherein the compound has the following Formula (III):

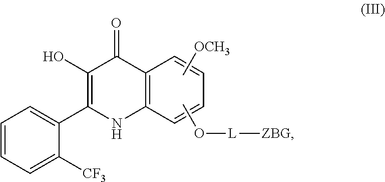

or a pharmaceutically acceptable salt thereof.

Another embodiment of the present disclosure is the compound of any one of the aforesaid embodiments, wherein the compound has the following Formula (IIIa):

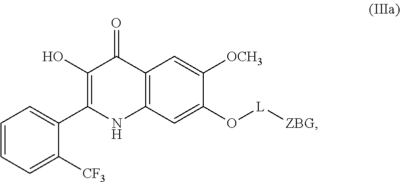

or a pharmaceutically acceptable salt thereof.

Another embodiment of the present disclosure is the compound of any one of the aforesaid embodiments, wherein the compound has the following Formula (IIIb):

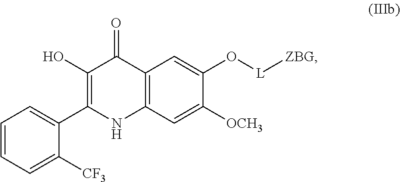

or a pharmaceutically acceptable salt thereof.

Another embodiment of the present disclosure is the compound of any one of the aforesaid embodiments, wherein the compound has the following Formula (IV):

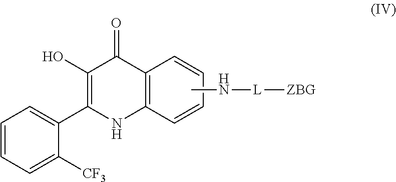

wherein Y is halogen, methyl, CF₃, OCH₃, OCH₂CH₃, OCH₂CH₂CH₃ or OCF₃,
or a pharmaceutically acceptable salt thereof. In some embodiments, Y is CF₃, F, CH₃, or OCH₃.

Another embodiment of the present disclosure is the compound of any one of the aforesaid embodiments, wherein the compound has the following Formula (IVa):

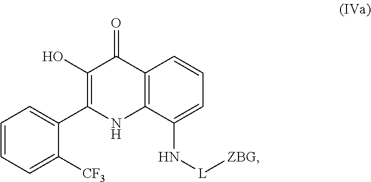

or a pharmaceutically acceptable salt thereof.

Another embodiment of the present disclosure is the compound of any one of the aforesaid embodiments, wherein the compound has the following Formula (IVb):

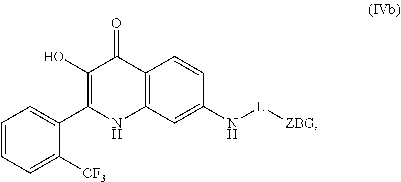

or a pharmaceutically acceptable salt thereof.

Another embodiment of the present disclosure is the compound of any one of the aforesaid embodiments, wherein the compound has the following Formula (IVc):

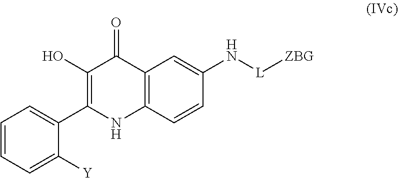

wherein Y is CF₃, F, CH₃, or OCH₃, or a pharmaceutically acceptable salt thereof.

Another embodiment of the present disclosure is the compound of any one of the aforesaid embodiments, wherein the compound has the following Formula (IVd):

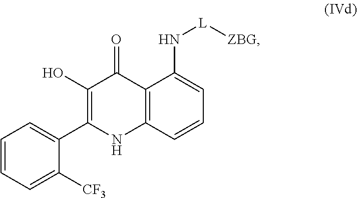

or a pharmaceutically acceptable salt thereof.

Another embodiment of the present disclosure is the compound of any one of the aforesaid embodiments, wherein the compound has the following Formula (V):

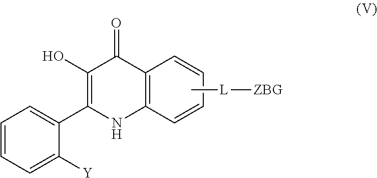

wherein Y is halogen, methyl, CF₃, OCH₃, OCH₂CH₃, OCH₂CH₂CH₃ or OCF₃,
or a pharmaceutically acceptable salt thereof. In some embodiments, Y is CF₃, F, CH₃, or OCH₃.

In some embodiments of the present disclosure, the compound of Formula (IIIa) is any one selected from the group consisting of:
N-hydroxy-2-((3-hydroxy-6-methoxy-4-oxo-2-(2-(trifluoromethyl)phenyl)-1,4-dihydroquinolin-7-yl)oxy)acetamide (Cpd007),
N-hydroxy-5-(2-((3-hydroxy-6-methoxy-4-oxo-2-(2-(trifluoromethyl)phenyl)-1,4-dihydroquinolin-7-yl)oxy)acetamido)pentanamide (Cpd009),
N-hydroxy-6-(2-((3-hydroxy-6-methoxy-4-oxo-2-(2-(trifluoromethyl)phenyl)-1,4-dihydroquinolin-7-yl)oxy)acetamido)hexanamide (Cpd010),
N-hydroxy-7-(2-((3-hydroxy-6-methoxy-4-oxo-2-(2-(trifluoromethyl)phenyl)-1,4-dihydroquinolin-7-yl)oxy)acetamido)heptanamide (Cpd011),
N-hydroxy-8-(2-((3-hydroxy-6-methoxy-4-oxo-2-(2-(trifluoromethyl)phenyl)-1,4-dihydroquinolin-7-yl)oxy)acetamido)octanamide (Cpd012),
N-(2-aminophenyl)-2-((3-hydroxy-6-methoxy-4-oxo-2-(2-(trifluoromethyl)phenyl)-1,4-dihydroquinolin-7-yl)oxy)acetamide (Cpd019), and
N-(2-amino-4-fluorophenyl)-2-((3-hydroxy-6-methoxy-4-oxo-2-(2-(trifluoromethyl)phenyl)-1,4-dihydroquinolin-7-yl)oxy)acetamide (Cpd020),
or a pharmaceutically acceptable salt thereof.

In some embodiments of the present disclosure, the compound of Formula (IIIb) is any one selected from the group consisting of:
N-hydroxy-2-((3-hydroxy-7-methoxy-4-oxo-2-(2-(trifluoromethyl)phenyl)-1,4-dihydroquinolin-6-yl)oxy)acetamide (Cpd008),
N-hydroxy-3-(2-((3-hydroxy-7-methoxy-4-oxo-2-(2-(trifluoromethyl)phenyl)-1,4-dihydroquinolin-6-yl)oxy)acetamido)propanamide (Cpd013),
N-hydroxy-4-(2-((3-hydroxy-7-methoxy-4-oxo-2-(2-(trifluoromethyl)phenyl)-1,4-dihydroquinolin-6-yl)oxy)acetamido)butanamide (Cpd014),
N-hydroxy-4-(2-((3-hydroxy-7-methoxy-4-oxo-2-(2-(trifluoromethyl)phenyl)-1,4-dihydroquinolin-6-yl)oxy)acetamido)pentanamide (Cpd015),
N-hydroxy-4-(2-((3-hydroxy-7-methoxy-4-oxo-2-(2-(trifluoromethyl)phenyl)-1,4-dihydroquinolin-6-yl)oxy)acetamido)hexanamide (Cpd016),
N-hydroxy-4-(2-((3-hydroxy-7-methoxy-4-oxo-2-(2-(trifluoromethyl)phenyl)-1,4-dihydroquinolin-6-yl)oxy)acetamido)heptanamide (Cpd017),
N-hydroxy-4-(2-((3-hydroxy-7-methoxy-4-oxo-2-(2-(trifluoromethyl)phenyl)-1,4-dihydroquinolin-6-yl)oxy)acetamido)octanamide (Cpd018),
N-(2-aminophenyl)-2-((3-hydroxy-7-methoxy-4-oxo-2-(2-(trifluoromethyl)phenyl)-1,4-dihydroquinolin-6-yl)oxy)acetamide (Cpd021),
N-(2-amino-4-fluorophenyl)-2-((3-hydroxy-7-methoxy-4-oxo-2-(2-(trifluoromethyl)phenyl)-1,4-dihydroquinolin-6-yl)oxy)acetamide (Cpd022),
N-(2-aminophenyl)-4-((2-((3-hydroxy-7-methoxy-4-oxo-2-(2-(trifluoromethyl)phenyl)-1,4-dihydroquinolin-6-yl)oxy)acetamido)methyl)benzamide (Cpd023),
N-(2-amino-4-fluorophenyl)-4-((2-((3-hydroxy-7-methoxy-4-oxo-2-(2-(trifluoromethyl)phenyl)-1,4-dihydroquinolin-6-yl)oxy)acetamido)methyl)benzamide (Cpd024),
N-hydroxy-4-((2-((3-hydroxy-7-methoxy-4-oxo-2-(2-(trifluoromethyl)phenyl)-1,4-dihydroquinolin-6-yl)oxy)acetamido)methyl)benzamide (Cpd025),
(E)-N-(2-aminophenyl)-3-(4-((2-((3-hydroxy-7-methoxy-4-oxo-2-(2-(trifluoromethyl)phenyl)-1,4-dihydroquinolin-6-yl)oxy)acetamido)methyl)phenyl)acrylamide (Cpd026), and
(E)-N-hydroxy-3-(4-((2-((3-hydroxy-7-methoxy-4-oxo-2-(2-(trifluoromethyl)phenyl)-1,4-dihydroquinolin-6-yl)oxy)acetamido)methyl)phenyl)acrylamide (Cpd027),
or a pharmaceutically acceptable salt thereof.

In some embodiments of the present disclosure, the compound of Formula (IVa) is any one selected from the group consisting of:
N¹-hydroxy-N⁶-(3-hydroxy-4-oxo-2-(2-(trifluoromethyl)phenyl)-1,4-dihydroquinolin-8-yl)adipamide (Cpd040),
N¹-hydroxy-N⁷-(3-hydroxy-4-oxo-2-(2-(trifluoromethyl)phenyl)-1,4-dihydroquinolin-8-yl)heptanediamide (Cpd041),
N¹-hydroxy-N¹-(3-hydroxy-4-oxo-2-(2-(trifluoromethyl)phenyl)-1,4-dihydroquinolin-8-yl)octanediamide (Cpd042),
N¹-hydroxy-N⁹-(3-hydroxy-4-oxo-2-(2-(trifluoromethyl)phenyl)-1,4-dihydroquinolin-8-yl)nonanediamide (Cpd043),
N-hydroxy-4-(2-((3-hydroxy-4-oxo-2-(2-(trifluoromethyl)phenyl)-1,4-dihydroquinolin-8-yl)amino)-2-oxoethyl)benzamide (Cpd047),
N-hydroxy-4-(((3-hydroxy-4-oxo-2-(2-(trifluoromethyl)phenyl)-1,4-dihydroquinolin-8-yl)amino)methyl)benzamide (Cpd050),
(E)-N-hydroxy-3-(4-(((3-hydroxy-4-oxo-2-(2-(trifluoromethyl)phenyl)-1,4-dihydroquinolin-8-yl)amino)methyl)phenyl)acrylamide (Cpd053), and
(E)-N-hydroxy-3-(3-(N-(3-hydroxy-4-oxo-2-(2-(trifluoromethyl)phenyl)-1,4-dihydroquinolin-8-yl)sulfamoyl)phenyl)acrylamide (Cpd057),
or a pharmaceutically acceptable salt thereof.

In some embodiments of the present disclosure, the compound of Formula (IVb) is any one selected from the group consisting of:
N¹-hydroxy-N⁶-(3-hydroxy-4-oxo-2-(2-(trifluoromethyl)phenyl)-1,4-dihydroquinolin-7-yl)adipamide (Cpd036),
N¹-hydroxy-N⁷-(3-hydroxy-4-oxo-2-(2-(trifluoromethyl)phenyl)-1,4-dihydroquinolin-7-yl)heptanediamide (Cpd037),
N¹-hydroxy-N¹-(3-hydroxy-4-oxo-2-(2-(trifluoromethyl)phenyl)-1,4-dihydroquinolin-7-yl)octanediamide (Cpd038),
N¹-hydroxy-N⁹-(3-hydroxy-4-oxo-2-(2-(trifluoromethyl)phenyl)-1,4-dihydroquinolin-7-yl)nonanediamide (Cpd039),
N-hydroxy-4-(2-((3-hydroxy-4-oxo-2-(2-(trifluoromethyl)phenyl)-1,4-dihydroquinolin-7-yl)amino)-2-oxoethyl)benzamide (Cpd046),
N-hydroxy-4-(((3-hydroxy-4-oxo-2-(2-(trifluoromethyl)phenyl)-1,4-dihydroquinolin-7-yl)amino)methyl)benzamide (Cpd049),
(E)-N-hydroxy-3-(4-(((3-hydroxy-4-oxo-2-(2-(trifluoromethyl)phenyl)-1,4-dihydroquinolin-7-yl)amino)methyl)phenyl)acrylamide (Cpd052),
(E)-N-hydroxy-3-(3-(N-(3-hydroxy-4-oxo-2-(2-(trifluoromethyl)phenyl)-1,4-dihydroquinolin-7-yl)sulfamoyl)phenyl)acrylamide (Cpd056),
(E)-N-hydroxy-3-(4-(N-(3-hydroxy-4-oxo-2-(2-(trifluoromethyl)phenyl)-1,4-dihydroquinolin-7-yl)sulfamoyl)phenyl)acrylamide (Cpd058),
N-hydroxy-3-(4-(N-(3-hydroxy-4-oxo-2-(2-(trifluoromethyl)phenyl)-1,4-dihydroquinolin-7-yl)sulfamoyl)phenyl)propanamide (Cpd059),
N-hydroxy-3-(N-(3-hydroxy-4-oxo-2-(2-(trifluoromethyl)phenyl)-1,4-dihydroquinolin-7-yl)sulfamoyl)benzamide (Cpd060),
N-hydroxy-4-(N-(3-hydroxy-4-oxo-2-(2-(trifluoromethyl)phenyl)-1,4-dihydroquinolin-7-yl)sulfamoyl)benzamide (Cpd061),
N¹-hydroxy-N⁴-(3-hydroxy-4-oxo-2-(2-(trifluoromethyl)phenyl)-1,4-dihydroquinolin-7-yl)terephthalamide (Cpd062), and
(E)-N-(3-hydroxy-4-oxo-2-(2-(trifluoromethyl)phenyl)-1,4-dihydroquinolin-7-yl)-4-(3-(hydroxyamino)-3-oxoprop-1-en-1-yl)benzamide (Cpd063),
or a pharmaceutically acceptable salt thereof.

In some embodiments of the present disclosure, the compound of Formula (IVc) is any one selected from the group consisting of:
N¹-hydroxy-N⁶-(3-hydroxy-4-oxo-2-(2-(trifluoromethyl)phenyl)-1,4-dihydroquinolin-6-yl)adipamide (Cpd032),
N¹-hydroxy-N⁷-(3-hydroxy-4-oxo-2-(2-(trifluoromethyl)phenyl)-1,4-dihydroquinolin-6-yl)heptanediamide (Cpd033),
N¹-hydroxy-N¹-(3-hydroxy-4-oxo-2-(2-(trifluoromethyl)phenyl)-1,4-dihydroquinolin-6-yl)octanediamide (Cpd034),
N¹-hydroxy-N⁹-(3-hydroxy-4-oxo-2-(2-(trifluoromethyl)phenyl)-1,4-dihydroquinolin-6-yl)nonanediamide (Cpd035),
N-hydroxy-4-(2-((3-hydroxy-4-oxo-2-(2-(trifluoromethyl)phenyl)-1,4-dihydroquinolin-6-yl)amino)-2-oxoethyl)benzamide (Cpd045),
N-hydroxy-4-(((3-hydroxy-4-oxo-2-(2-(trifluoromethyl)phenyl)-1,4-dihydroquinolin-6-yl)amino)methyl)benzamide (Cpd048),
(E)-N-hydroxy-3-(4-(((3-hydroxy-4-oxo-2-(2-(trifluoromethyl)phenyl)-1,4-dihydroquinolin-6-yl)amino)methyl)phenyl)acrylamide (Cpd051),
(E)-N-hydroxy-3-(3-(N-(3-hydroxy-4-oxo-2-(2-(trifluoromethyl)phenyl)-1,4-dihydroquinolin-6-yl)sulfamoyl)phenyl)acrylamide (Cpd055),
N¹-(2-(2-fluorophenyl)-3-hydroxy-4-oxo-1,4-dihydroquinolin-6-yl)-N¹-hydroxyoctanediamide (Cpd069), and
N¹-hydroxy-N¹-(3-hydroxy-4-oxo-2-(o-tolyl)-1,4-dihydroquinolin-6-yl)octanediamide (Cpd070),
or a pharmaceutically acceptable salt thereof.

In some embodiments of the present disclosure, the compound of Formula (IVd) is any one selected from the group consisting of:
N¹-hydroxy-N⁶-(3-hydroxy-4-oxo-2-(2-(trifluoromethyl)phenyl)-1,4-dihydroquinolin-5-yl)adipamide (Cpd028),
N¹-hydroxy-N⁷-(3-hydroxy-4-oxo-2-(2-(trifluoromethyl)phenyl)-1,4-dihydroquinolin-5-yl)heptanediamide (Cpd029),
N¹-hydroxy-N¹-(3-hydroxy-4-oxo-2-(2-(trifluoromethyl)phenyl)-1,4-dihydroquinolin-5-yl)octanediamide (Cpd030),
N¹-hydroxy-N⁹-(3-hydroxy-4-oxo-2-(2-(trifluoromethyl)phenyl)-1,4-dihydroquinolin-5-yl)nonanediamide (Cpd031),
N-hydroxy-4-(2-((3-hydroxy-4-oxo-2-(2-(trifluoromethyl)phenyl)-1,4-dihydroquinolin-5-yl)amino)-2-oxoethyl)benzamide (Cpd044), and
(E)-N-hydroxy-3-(3-(N-(3-hydroxy-4-oxo-2-(2-(trifluoromethyl)phenyl)-1,4-dihydroquinolin-5-yl)sulfamoyl)phenyl)acrylamide (Cpd054),
or a pharmaceutically acceptable salt thereof.

In some embodiments of the present disclosure, the compound of Formula (V) is any one selected from the group consisting of:
(E)-N-hydroxy-3-(3-hydroxy-4-oxo-2-(2-(trifluoromethyl)phenyl)-1,4-dihydroquinolin-6-yl)acrylamide (Cpd064),
(E)-N-hydroxy-3-(3-hydroxy-4-oxo-2-(2-(trifluoromethyl)phenyl)-1,4-dihydroquinolin-7-yl)acrylamide (Cpd065),
(E)-3-(2-(2-fluorophenyl)-3-hydroxy-4-oxo-1,4-dihydroquinolin-6-yl)-N-hydroxyacrylamide (Cpd066),
(E)-N-hydroxy-3-(3-hydroxy-2-(2-methoxyphenyl)-4-oxo-1,4-dihydroquinolin-6-yl)acrylamide (Cpd067), and
(E)-N-hydroxy-3-(3-hydroxy-4-oxo-2-(o-tolyl)-1,4-dihydroquinolin-6-yl)acrylamide (Cpd068),
or a pharmaceutically acceptable salt thereof.

One embodiment of the present disclosure is the compound of Formula (IIb) or a pharmaceutically acceptable salt thereof,

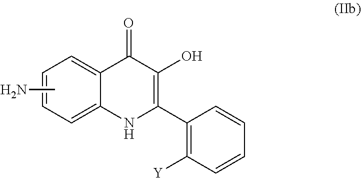

wherein Y is halogen, methyl, CF₃, OCH₃, OCH₂CH₃, OCH₂CH₂CH₃ or OCF₃. In some embodiments, Y is CF₃, F, CH₃, or OCH₃.

In some embodiments of the present disclosure, the compound of Formula (IIb) is any one selected from the group consisting of:
5-amino-3-hydroxy-2-(2-(trifluoromethyl)phenyl)quinolin-4(1H)-one (Cpd003),
6-amino-3-hydroxy-2-(2-(trifluoromethyl)phenyl)quinolin-4(1H)-one (Cpd004),
7-amino-3-hydroxy-2-(2-(trifluoromethyl)phenyl)quinolin-4(1H)-one (Cpd005),
8-amino-3-hydroxy-2-(2-(trifluoromethyl)phenyl)quinolin-4(1H)-one (Cpd006),
6-amino-2-(2-fluorophenyl)-3-hydroxyquinolin-4(1H)-one (58a), and
6-amino-3-hydroxy-2-(o-tolyl)quinolin-4(1H)-one (58b),
or a pharmaceutically acceptable salt thereof.

Also within the present disclosure is a pharmaceutical composition, comprising: (1) the compound of the present disclosure, or a pharmaceutically acceptable salt thereof; and (2) at least one pharmaceutically acceptable carrier.

The “carrier” helps for introducing the compound into cells or tissues. It includes, but is not limited to, stabilizers, diluents, suspending agents, thickeners, and/or excipients.

The “pharmaceutical compositions” herein may be prepared in a manner well known in the pharmaceutical field, and they may be administered or applied by various routes, depending on whether local or systemic treatment is required and the area to be treated. It may be topically administered (for example, transdermal, skin, eye and mucous membranes including intranasal, vaginal and rectal delivery), pulmonary administered (for example, by inhalation or insufflation of powder or aerosol, including through sprayers; intratracheal, intranasal), orally or parenterally administered. Parenteral administration includes intravenous, intraarterial, subcutaneous, intraperitoneal or intramuscular injection or infusion; or intracranial, such as intrathecal or intracerebroventricular administration. It may be administered parenterally in a single bolus dose, or it may be administered by, for example, a continuous infusion vacuum. The pharmaceutical composition herein includes but is not limited to the following forms: tablets, pills, powders, lozenges, sachets, cachets, elixirs, suspensions, emulsions, solutions, syrups, aerosols (solid or dissolved in a liquid vehicle); for example, ointments, soft and hard gelatin capsules, suppositories, sterile injection solutions and sterile packaged powders.

Also within the present disclosure is a method for treating a cancer, which includes the step of administering to the subject in need thereof an effective amount of the compound of the present disclosure or a pharmaceutically acceptable salt thereof.

Further covered by the present disclosure a method of inhibiting a growth of tumor cells, which includes the step of administering to a subject in need thereof an effective amount of the compound of the present disclosure or a pharmaceutically acceptable salt thereof.

In the present disclosure, the compound of the present disclosure or a pharmaceutically acceptable salt thereof can inhibit the growth of tumor cells to achieve the purpose of treating a cancer. Examples of the cancer include, but are not limited to, gastric cancer, colon cancer, colorectal cancer, breast cancer, lung cancer, prostate cancer, bladder cancer, pancreatic cancer, liver cancer, uterine cancer, cervical cancer, endometrial cancer, esophageal cancer, leukemia, lymphoma, kidney cancer, osteosarcoma, ovarian cancer, skin cancer, small intestine cancer, thymus cancer, thyroid cancer, nervous system cancers, bone cancer, brain cancer, or head and neck cancer.

As used herein, the term “compound” is meant to include all stereoisomers, geometric isomers, tautomers, and isotopes of the structures depicted. Compounds herein identified by name or structure as one particular tautomeric form are intended to include other tautomeric forms unless otherwise specified.

As used herein, the term “moiety” refers to a specific segment or functional group of a molecule. Chemical moieties are often recognized chemical entities embedded in or appended to a molecule. Typically, the term is used to describe the larger and characteristic parts of organic molecules.

As used herein, the term “alkyl” refers to a straight or branched hydrocarbon chain radical consisting solely of carbon and hydrogen atoms, containing no unsaturation, having from one to ten carbon atoms (e.g., C₁₋₁₀ alkyl, C₁₋₆ alkyl or C₁₋₃ alkyl). Whenever it appears herein, a numerical range such as “1 to 10” refers to each integer in the given range—e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl, tertiary butyl, pentyl, isopentyl, neopentyl, hexyl, heptyl, octyl, nonyl and decyl.

As used herein, the term “alkoxy” refers to an —O-alkyl group. Examples include methoxy, ethoxy, propoxy, and isopropoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, an asterisk “*” is used to designate the point of attachment for any radical group or substituent group.

As used herein, the term “ortho-aminoanilide” refers to a moiety having the structure

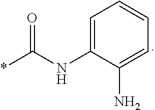 [SMILES: *C(=O)Nc1ccccc1N]

As used herein, the term “composition” or “pharmaceutical composition” refers to a mixture of at least one compound useful within the invention with a pharmaceutically acceptable carrier or excipient. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system.

An appropriate therapeutic amount in any individual case may be determined by one of ordinary skill in the art using routine experimentation. Effective amounts vary, as recognized by those skilled in the art, depending on route of administration, excipient usage, and the possibility of co-usage with other therapeutic treatments such as use of other active agents.

As used herein, the term “pharmaceutically acceptable” refers to a material, such as a carrier or excipient, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of the disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

The term “subject” as used herein refers to a mammal (e.g., human, monkey, dog, feline, rabbit, rat, mouse). Preferably, the subject is a human.

The term “treating” refers to application or administration of the compound to a subject with the purpose to cure, alleviate, relieve, alter, remedy, improve, or affect the disease, the symptom, or the predisposition.

The following embodiments are made to clearly exhibit the above-mentioned and other technical contents, features and/or effects of the present disclosure. Through the exposition by means of the specific embodiments, people would further understand the technical means and effects the present disclosure adopts to achieve the above-indicated objectives. Moreover, as the contents disclosed herein should be readily understood and can be implemented by a person skilled in the art, all equivalent changes or modifications which do not depart from the concept of the present disclosure should be encompassed by the appended claims.

### EXAMPLE

Without further elaboration, it is believed that one skilled in the art can, based on the above description, utilize the present disclosure to its fullest extent. The following specific examples are therefore to be construed as merely illustrative, and not limitative of the remainder of the disclosure in any way whatsoever. All publications cited herein are incorporated by reference in their entirety.

Described below are the procedures used to synthesize the exemplary compounds of the present disclosure.

### Example 1

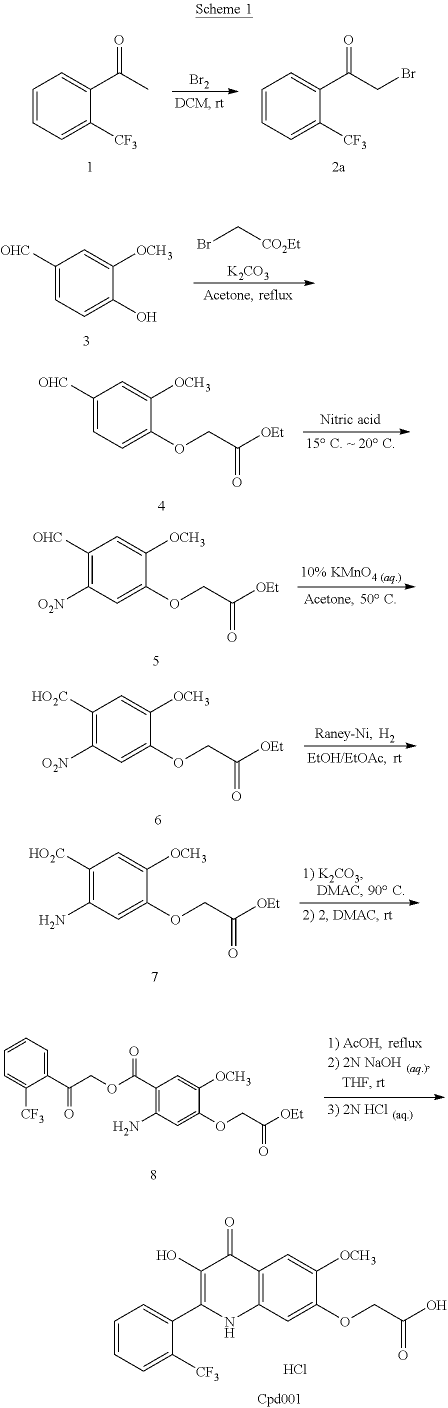

### Preparation of 2-bromo-1-(2-(trifluoromethyl)phenyl)ethan-1-one (2a)

To a stirred solution of 1-(2-(trifluoromethyl)phenyl)ethan-1-one (1, 9.0 g, 48 mmol) in 60 mL of DCM (dichloromethane) was added dropwise a solution of bromine (2.5 mL, 48 mmol) in 20 mL of DCM at 20° C. The reaction mixture was allowed to stir for 1 h at 20° C. Upon completion, the reaction was quenched by addition of 1 M Na₂SO₃ aqueous solution. The organic layer was separated, washed with saturated NaHCO₃ aqueous solution, brine, dried over anhydrous MgSO₄, filtered, and concentrated under reduced pressure. The crude product was obtained as a clear oil (12.3 g) containing the desired 2-bromo-1-(2-(trifluoromethyl)phenyl)ethan-1-one (2a) along with unreacted starting material and over-brominated products. This crude mixture was used directly in subsequent reactions without further purification.

### Preparation of ethyl 2-(4-formyl-2-methoxyphenoxy)acetate (4)

A mixture of vanillin (3, 20.0 g, 0.13 mol), ethyl bromoacetate (15 mL, 0.14 mol) and K₂CO₃ (37.0 g, 0.26 mol) in 200 mL of acetone was refluxed for 24 hours. The reaction mixture was then allowed to cool to room temperature and filtered. The filtrate was concentrated under reduced pressure, and the residue was dissolved in EtOAc (ethyl acetate). The organic layer was washed with water, brine, dried over anhydrous MgSO₄, filtered, and concentrated under reduced pressure. The crude product was washed with hexanes to afford the desired product (4) as a pale yellow solid without further purification (30.6 g, 98% yield).

### Preparation of ethyl 2-(4-formyl-2-methoxy-5-nitrophenoxy)acetate (5)

To a chilled 70% nitric acid (150 mL) in an ice bath, ethyl 2-(4-formyl-2-methoxyphenoxy)acetate (4, 30.6 g, 0.13 mol) was added portionwise. The reaction solution was then stirred at 15-20° C. for 30 minutes. Upon completion, the reaction mixture was poured into 1.5 L of ice-cold water. The resulting precipitate was collected by filtration, rinsed with deionized water, and dried under vacuum. The brown solid was recrystallized from hot EtOAc to afford the desired product (5) as a yellow solid (18.1 g, 50% yield).

### Preparation of 4-(2-ethoxy-2-oxoethoxy)-5-methoxy-2-nitrobenzoic acid (6)

To a stirred solution of ethyl 2-(4-formyl-2-methoxy-5-nitrophenoxy)acetate (5, 9.0 g, 32 mmol) in 150 mL of acetone was dropwise added 10% KMnO₄ aqueous solution (100 mL, 64 mmol) at room temperature. The reaction mixture was stirred at 50° C. for 1 h, and then the reaction mixture was filtered through a Celite® pad. The pad was washed with 400 mL of hot water. The filtrate was concentrated under reduced pressure to remove acetone, and the resulting aqueous solution was treated with NaHSO₃ and acidified to pH 3 with 2 N HCl aqueous solution. The precipitated product was collected by filtration, washed with deionized water, and dried under vacuum to afford the desired product (6) as a beige solid (13.1 g, 69% yield).

### Preparation of 2-amino-4-(2-ethoxy-2-oxoethoxy)-5-methoxybenzoic acid (7)

A suspension of 4-(2-ethoxy-2-oxoethoxy)-5-methoxy-2-nitrobenzoic acid (6, 13.1 g, 44 mmol) and Raney nickel (13 g) in a 1:1 mixture of EtOH and EtOAc (300 mL) was stirred at room temperature for 24 hours under hydrogen atmosphere. The reaction mixture was filtered through a Celite® pad, and the filtrate was concentrated under reduced pressure to afford the desired product (7) as a brown solid (10.3 g, 87% yield).

### Preparation of 2-oxo-2-(2-(trifluoromethyl)phenyl)ethyl 2-amino-4-(2-ethoxy-2-oxoethoxy)-5-methoxybenzoate (8)

A mixture of 2-amino-4-(2-ethoxy-2-oxoethoxy)-5-methoxybenzoic acid (7, 10.3 g, 38 mmol) and finely ground K₂CO₃ (5.6 g, 40 mmol) in 60 mL of DMAC (N,N-dimethylacetamide) was stirred at 90° C. for 1 h. After cooling to 0° C., 2-bromo-1-(2-(trifluoromethyl)phenyl)ethan-1-one (2, 12.8 g, 48 mmol) was added. The reaction mixture was stirred at room temperature for 1 h and then poured into ice-cold water. The resulting precipitate was collected by filtration, washed with deionized water, 20% EtOAc in hexanes, and dried under vacuum to afford the desired product (8) as a brown solid (15.5 g, 89% yield).

### Preparation of 2-((3-hydroxy-6-methoxy-4-oxo-2-(2-(trifluoromethyl)phenyl)-1,4-dihydroquinolin-7-yl)oxy)acetic acid hydrochloride (Cpd001)

A solution of 2-oxo-2-(2-(trifluoromethyl)phenyl)ethyl 2-amino-4-(2-ethoxy-2-oxoethoxy)-5-methoxybenzoate (8, 15.5 g, 34 mmol) in 120 mL of glacial acetic acid was refluxed for 24 hours. Upon completion, the reaction mixture was concentrated under reduced pressure to remove acetic acid. The residue was dissolved into 20 mL of THF (tetrahydrofuran), and 40 mL of 2 N NaOH aqueous solution was added dropwise at an ice bath. The mixture was stirred at room temperature for 24 h and then concentrated under reduced pressure to remove THF. The resulting suspension was acidified to pH 2 by dropwise addition of 2 N HCl aqueous solution at an ice bath. The precipitate was collected by filtration, washed with deionized water, and dried under vacuum. The residue was dispersed in EtOAc, filtered, and dried under vacuum to afford the desired product (Cpd001) as a beige solid (9.1 g, 60% yield).

### Example 2

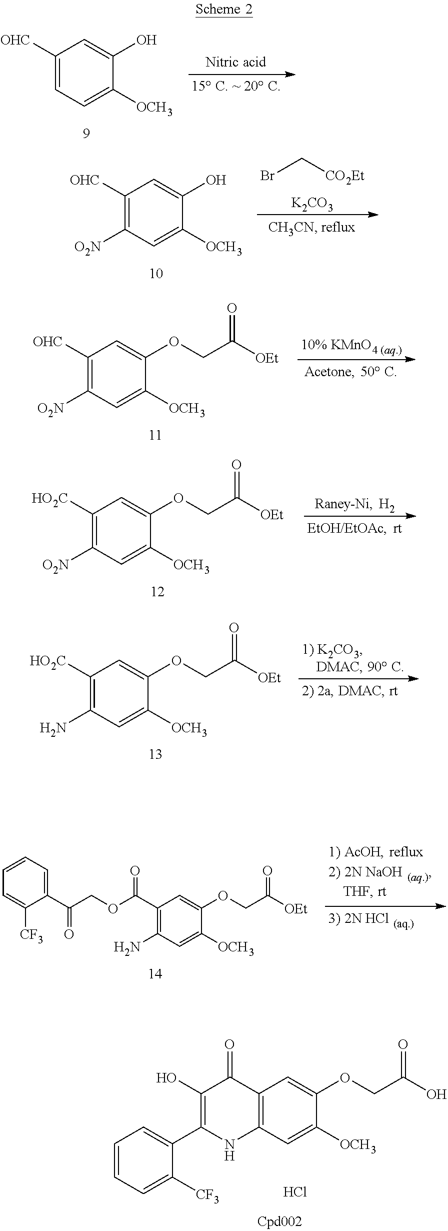

### Preparation of 5-hydroxy-4-methoxy-2-nitrobenzaldehyde (10)

To a stirred mixture of 3-hydroxy-4-methoxybenzaldehyde (9, 40.0 g, 0.26 mol) and 40 μL of concentrated H₂SO₄ in 140 mL of glacial acetic acid was added dropwise 11.5 mL (0.26 mol) of fuming nitric acid over 40 minutes, maintaining the temperature below 20° C. The reaction mixture was stirred at 15-20° C. for 1 h. Upon completion, the reaction mixture was poured into 1.5 L of ice-cold water. The resulting precipitate was collected by filtration, rinsed with deionized water, and dried under vacuum. The brown solid was purified by flash chromatography (40% acetone in hexanes, R_{f}=0.5), followed by recrystallization from hot EtOAc to afford the desired product (10) as a yellow solid (12.5 g, 24% yield). The orange solid byproduct, 3-hydroxy-4-methoxy-2-nitrobenzaldehyde, was also isolated (17.5 g, 34% yield).

### Preparation of ethyl 2-(5-formyl-2-methoxy-4-nitrophenoxy)acetate (11)

A mixture of 5-hydroxy-4-methoxy-2-nitrobenzaldehyde (10, 13.4 g, 68 mmol), ethyl bromoacetate (7.9 mL, 71 mmol) and K₂CO₃ (18.8 g, 136 mmol) in 200 mL of acetonitrile was refluxed for 24 hours. The reaction mixture was then allowed to cool to room temperature and filtered. The filtrate was concentrated under reduced pressure, and the residue was dissolved in EtOAc. The organic layer was washed with water, brine, dried over anhydrous MgSO₄, filtered, and concentrated under reduced pressure. The crude product was washed with hexanes to afford the desired product (11) as a yellow solid without further purification (18.5 g, 96% yield).

### Preparation of 5-(2-ethoxy-2-oxoethoxy)-4-methoxy-2-nitrobenzoic acid (12)

To a stirred solution of ethyl 2-(5-formyl-2-methoxy-4-nitrophenoxy)acetate (11, 9.0 g, 32 mmol) in 150 mL of acetone was dropwise added 10% KMnO₄ aqueous solution (100 mL, 64 mmol) at room temperature. The reaction mixture was stirred at 50° C. for 1 h, and then the reaction mixture was filtered through a Celite® pad. The pad was washed with 400 mL of hot water. The filtrate was concentrated under reduced pressure to remove acetone, and the resulting aqueous solution was treated with NaHSO₃ and acidified to pH 3 with 2 N HCl aqueous solution. The precipitated product was collected by filtration, washed with deionized water, and dried under vacuum to afford the desired product (12) as a white solid (7.4 g, 78% yield).

### Preparation of 2-amino-5-(2-ethoxy-2-oxoethoxy)-4-methoxybenzoic acid (13)

A suspension of 5-(2-ethoxy-2-oxoethoxy)-4-methoxy-2-nitrobenzoic acid (12, 14.6 g, 49 mmol) and Raney nickel (15 g) in a 4:1 mixture of EtOH and EtOAc (500 mL) was stirred at room temperature for 24 hours under hydrogen atmosphere. The reaction mixture was filtered through a Celite® pad, and the filtrate was concentrated under reduced pressure to afford the desired product (13) as a brown solid (11.0 g, 84% yield).

### Preparation of 2-oxo-2-(2-(trifluoromethyl)phenyl)ethyl 2-amino-5-(2-ethoxy-2-oxoethoxy)-4-methoxybenzoate (14)

A mixture of 2-amino-5-(2-ethoxy-2-oxoethoxy)-4-methoxybenzoic acid (13, 11.0 g, 41 mmol) and finely ground K₂CO₃ (5.8 g, 43 mmol) in 60 mL of DMAC was stirred at 90° C. for 1 h. After cooling to 0° C., 2-bromo-1-(2-(trifluoromethyl)phenyl)ethan-1-one (2a, 13.7 g, 51 mmol) was added. The reaction mixture was stirred at room temperature for 1 h and then poured into ice-cold water. The resulting precipitate was collected by filtration, washed with deionized water, 20% EtOAc in hexanes, and dried under vacuum to afford the desired product (14) as a black oil which was used directly in subsequent reactions without further purification (20.7 g, >99% yield).

### Preparation of 2-((3-hydroxy-7-methoxy-4-oxo-2-(2-(trifluoromethyl)phenyl)-1,4-dihydroquinolin-6-yl)oxy)acetic acid hydrochloride (Cpd002)

A solution of 2-oxo-2-(2-(trifluoromethyl)phenyl)ethyl 2-amino-5-(2-ethoxy-2-oxoethoxy)-4-methoxybenzoate (14, 20.0 g, 44 mmol) in 150 mL of glacial acetic acid was refluxed for 24 hours. Upon completion, the reaction mixture was concentrated under reduced pressure to remove acetic acid. The residue was dissolved into 20 mL of THF, and 40 mL of 2 N NaOH aqueous solution was added dropwise at an ice bath. The mixture was stirred at room temperature for 5 h and then concentrated under reduced pressure to remove THF. The resulting suspension was acidified to pH 2 by dropwise addition of 2 N HCl aqueous solution at an ice bath. The precipitate was collected by filtration, washed with deionized water, and dried under vacuum. The residue was dispersed in EtOAc, filtered, and dried under vacuum to afford the desired product (Cpd002) as a beige solid (8.6 g, 47% yield).

### Example 3

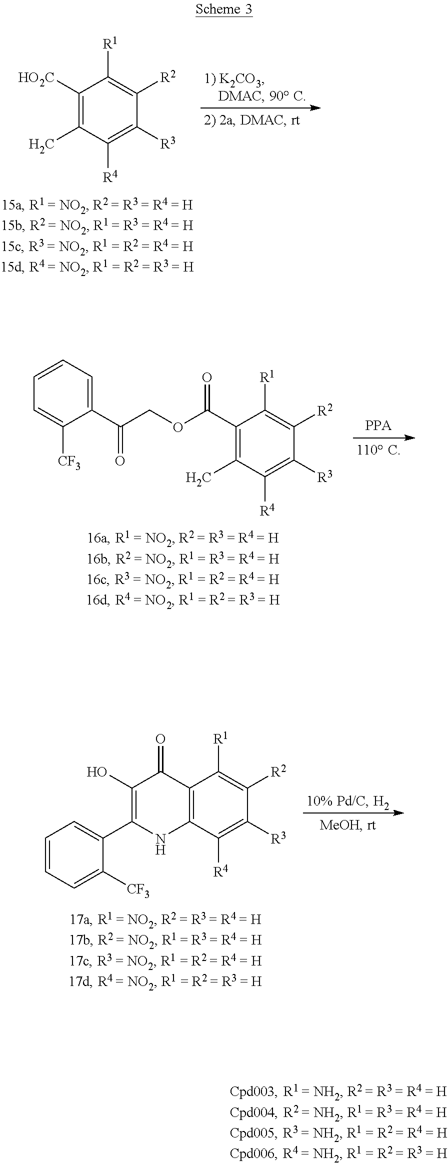

### Preparation of 2-oxo-2-(2-(trifluoromethyl)phenyl)ethyl 2-amino-6-nitrobenzoate (16a)

A mixture of 2-amino-6-nitrobenzoic acid (15a, 2.0 g, 11 mmol) and finely ground K₂CO₃ (1.6 g, 12 mmol) in 20 mL of DMAC was stirred at 90° C. for 1 h. After cooling to 0° C., 2-bromo-1-(2-(trifluoromethyl)phenyl)ethan-1-one (2a, 3.7 g, 14 mmol) was added. The reaction mixture was stirred at room temperature for 1 h and then poured into ice-cold water. The resulting precipitate was collected by filtration and dissolved in EtOAc. The organic layer was washed with deionized water, brine, dried over anhydrous MgSO₄, filtered, and concentrated under reduced pressure to afford the desired product (16a) as a yellow solid which was used directly in subsequent reactions without further purification (3.8 g, 94% yield).

### Preparation of 2-oxo-2-(2-(trifluoromethyl)phenyl)ethyl 2-amino-5-nitrobenzoate (16b)

The procedure for 16b was identical to that described for 16a, except that 2-amino-5-nitrobenzoic acid (15b, 3.0 g, 16 mmol) was used as the starting material. The desired product (16b) was obtained as a yellow solid (5.5 g, 91% yield).

### Preparation of 2-oxo-2-(2-(trifluoromethyl)phenyl)ethyl 2-amino-4-nitrobenzoate (16c)

The procedure for 16c was identical to that described for 16a, except that 2-amino-4-nitrobenzoic acid (15c, 3.0 g, 16 mmol) was used as the starting material. The desired product (16c) was obtained as an orange solid (6.0 g, 99% yield).

### Preparation of 2-oxo-2-(2-(trifluoromethyl)phenyl)ethyl 2-amino-3-nitrobenzoate (16d)

The procedure for 16d was identical to that described for 16a, except that 2-amino-3-nitrobenzoic acid (15d, 3.0 g, 16 mmol) was used as the starting material. The desired product (16d) was obtained as a yellow solid (3.7 g, 91% yield).

### Preparation of 3-hydroxy-5-nitro-2-(2-(trifluoromethyl)phenyl)quinolin-4(1H)-one (17a)

A mixture of 2-oxo-2-(2-(trifluoromethyl)phenyl)ethyl 2-amino-6-nitrobenzoate (16a, 3.8 g, 10 mmol) and 20 mL of PPA (polyphosphoric acid) was stirred at 110° C. for 3 hours. Upon completion, the reaction mixture was poured into 200 mL of ice-cold water and the precipitate was collected by filtration. The solid was rinsed with deionized water and dried under reduced pressure to afford the desired product (17a) as a brown solid (3.4 g, 94% yield).

### Preparation of 3-hydroxy-6-nitro-2-(2-(trifluoromethyl)phenyl)quinolin-4(1H)-one (17b)

The procedure for 17b was identical to that described for 17a, except that 2-oxo-2-(2-(trifluoromethyl)phenyl)ethyl 2-amino-5-nitrobenzoate (16b, 5.5 g, 15 mmol) was used as the starting material. The desired product (17b) was obtained as a yellow solid (5.0 g, 96% yield).

### Preparation of 3-hydroxy-7-nitro-2-(2-(trifluoromethyl)phenyl)quinolin-4(1H)-one (17c)

The procedure for 17c was identical to that described for 17a, except that 2-oxo-2-(2-(trifluoromethyl)phenyl)ethyl 2-amino-4-nitrobenzoate (16c, 3.0 g, 8 mmol) was used as the starting material. The desired product (17c) was obtained as a yellow solid (2.7 g, 95% yield).

### Preparation of 3-hydroxy-7-nitro-2-(2-(trifluoromethyl)phenyl)quinolin-4(1H)-one (17d)

The procedure for 17d was identical to that described for 17a, except that 2-oxo-2-(2-(trifluoromethyl)phenyl)ethyl 2-amino-3-nitrobenzoate (16d, 3.7 g, 10 mmol) was used as the starting material. The desired product (17d) was obtained as a brown solid (3.4 g, 99% yield).

### Preparation of 5-amino-3-hydroxy-2-(2-(trifluoromethyl)phenyl)quinolin-4(1H)-one (Cpd003)

A suspension of 3-hydroxy-5-nitro-2-(2-(trifluoromethyl)phenyl)quinolin-4(1H)-one (17a, 3.4 g, 10 mmol) and 10% Pd/C (680 mg, 50% wet) in 250 mL of MeOH was stirred at room temperature for 3 hours under a H₂ atmosphere. Upon completion, the reaction mixture was filtered through a Celite® pad. The filtrate was concentrated under reduced pressure to afford the desired product (Cpd003) as a brown solid (3.1 g, 99% yield).

### Preparation of 6-amino-3-hydroxy-2-(2-(trifluoromethyl)phenyl)quinolin-4(1H)-one (Cpd004)

The procedure for Cpd004 was identical to that described for Cpd003, except that 3-hydroxy-6-nitro-2-(2-(trifluoromethyl)phenyl)quinolin-4(1H)-one (17b, 2.0 g, 6 mmol) was used as the starting material. The desired product (Cpd004) was obtained as a gray solid (1.8 g, 99% yield).

### Preparation of 7-amino-3-hydroxy-2-(2-(trifluoromethyl)phenyl)quinolin-4(1H)-one (Cpd005)

The procedure for Cpd005 was identical to that described for Cpd003, except that 3-hydroxy-7-nitro-2-(2-(trifluoromethyl)phenyl)quinolin-4(1H)-one (17c, 2.7 g, 8 mmol) was used as the starting material. The desired product (Cpd005) was obtained as a gray solid (2.5 g, 99% yield).

### Preparation of 8-amino-3-hydroxy-2-(2-(trifluoromethyl)phenyl)quinolin-4(1H)-one (Cpd006)

The procedure for Cpd006 was identical to that described for Cpd003, except that 3-hydroxy-8-nitro-2-(2-(trifluoromethyl)phenyl)quinolin-4(1H)-one (17d, 3.4 g, 10 mmol) was used as the starting material. The desired product (Cpd006) was obtained as a gray solid (3.0 g, 96% yield).

### Example 4

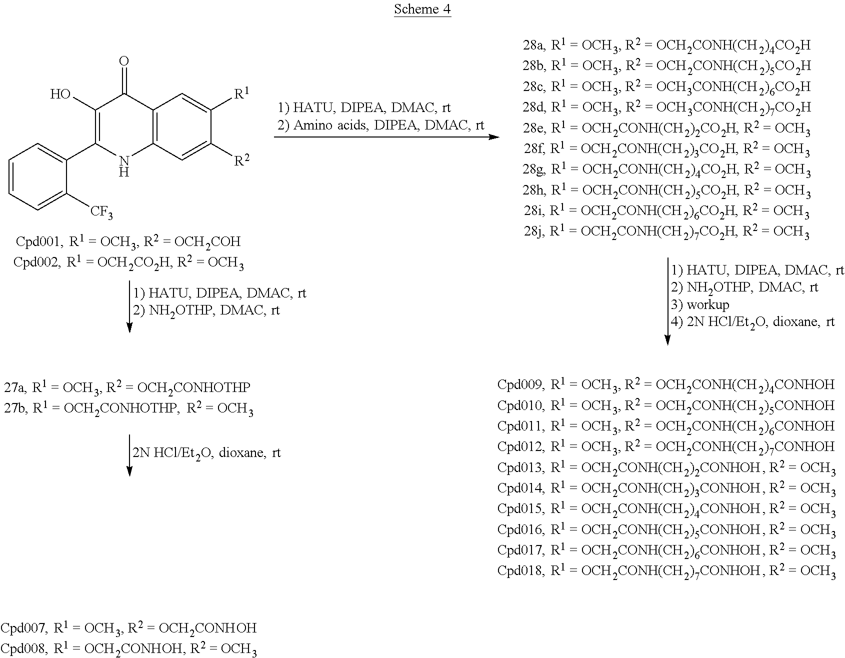

### Preparation of 2-((3-hydroxy-6-methoxy-4-oxo-2-(2-(trifluoromethyl)phenyl)-1,4-dihydroquinolin-7-yl)oxy)-N-((tetrahydro-2H-pyran-2-yl)oxy)acetamide (27a)

A mixture of 2-((3-hydroxy-6-methoxy-4-oxo-2-(2-(trifluoromethyl)phenyl)-1,4-dihydroquinolin-7-yl)oxy)acetic acid hydrochloride (Cpd001, 100 mg, 0.22 mmol), DIPEA (N,N-diisopropylethylamine)(38 μL, 0.22 mmol) and HATU (Hexafluorophosphate Azabenzotriazole Tetramethyl Uronium)(94 mg, 0.25 mmol) in 1 mL of DMAC was stirred at room temperature for 30 minutes. Subsequently, a solution of NH₂OTHP (32 mg, 0.27 mmol) in 1 mL of DMAC was added. The mixture was stirred at room temperature for 5 hours. Upon completion, the reaction mixture was poured into 20 mL of cold water. The resulting white precipitate was collected by filtration, rinsed with deionized water, and dried under vacuum. The crude product was purified by flash chromatography using a gradient elution of 0% to 20% MeOH in DCM. The desired product (27a) was obtained as a beige solid (34 mg, 30% yield).

### Preparation of 2-((3-hydroxy-7-methoxy-4-oxo-2-(2-(trifluoromethyl)phenyl)-1,4-dihydroquinolin-6-yl)oxy)-N-((tetrahydro-2H-pyran-2-yl)oxy)acetamide (27b)

The procedure for 27b was identical to that described for 27a, except that Cpd002 (100 mg, 0.22 mmol) was used as the starting material. The desired product (27b) was obtained as a brown solid (21 mg, 18% yield).

### Preparation of N-hydroxy-2-((3-hydroxy-6-methoxy-4-oxo-2-(2-(trifluoromethyl)phenyl)-1,4-dihydroquinolin-7-yl)oxy)acetamide (Cpd007)

To a stirred solution of 27a (34 mg, 0.07 mmol) in 2 mL of 1,4-dioxane was added 2 mL of 2 N HCl in dimethyl ether solution dropwise at room temperature. The reaction mixture was stirred at room temperature for 1 h. Upon completion, the mixture was concentrated under reduced pressure and the residue was washed with EtOAc to afford the desired product (Cpd007) as a white solid (27 mg, 95% yield). ¹H NMR (500 MHz, DMSO-d6) δ 13.41-12.57 (m, 2H), 10.90 (s, 1H), 8.91 (br s, 1H), 7.96 (d, J=7.9 Hz, 1H), 7.86 (d, J=7.4 Hz, 1H), 7.82 (d, J=7.3 Hz, 1H), 7.68 (d, J=7.0 Hz, 1H), 7.60 (s, 1H), 7.06 (s, 1H), 4.59 (s, 2H), 3.93 (s, 3H).

### Preparation of N-hydroxy-2-((3-hydroxy-7-methoxy-4-oxo-2-(2-(trifluoromethyl)phenyl)-1,4-dihydroquinolin-6-yl)oxy)acetamide (Cpd008)

The procedure for Cpd008 was identical to that described for Cpd007, except that 27b (21 mg, 0.04 mmol) was used as the starting material. The desired product (Cpd008) was obtained as an white solid (17 mg, 89% yield). ¹H NMR (500 MHz, DMSO-d6) δ 12.55-12.29 (m, 2H), 10.87 (s, 1H), 8.58 (br s, 1H), 7.95 (d, J=7.8 Hz, 1H), 7.86 (t, J=7.6 Hz, 1H), 7.79 (t, J=7.7 Hz, 1H), 7.66 (d, J=7.4 Hz, 1H), 7.50 (s, 1H), 7.05 (s, 1H), 4.57 (s, 2H), 3.87 (s, 3H).

### Preparation of 5-(2-((3-hydroxy-6-methoxy-4-oxo-2-(2-(trifluoromethyl)phenyl)-1,4-dihydroquinolin-7-yl)oxy)acetamido)pentanoic acid (28a)

A mixture of 2-((3-hydroxy-6-methoxy-4-oxo-2-(2-(trifluoromethyl)phenyl)-1,4-dihydroquinolin-7-yl)oxy)acetic acid hydrochloride (Cpd001, 100 mg, 0.22 mmol), DIPEA (40 μL, 0.22 mmol) and HATU (94 mg, 0.25 mmol) in 1 mL of DMAC was stirred at room temperature for 30 minutes. Subsequently, a mixture of 5-aminopentanoic acid (79 mg, 0.67 mmol) and DIPEA (115 μL, 0.67 mmol) in 1 mL of DMAC was added. The mixture was stirred at room temperature for 16 hours. Upon completion, the reaction mixture was poured into 20 mL of cold water and acidified with 2 N HCl aqueous solution to pH 4. The resulting white precipitate was collected by filtration, rinsed with deionized water, and dried under vacuum to afford the desired product (28a) as a beige solid (82 mg, 67% yield).

### Preparation of 6-(2-((3-hydroxy-6-methoxy-4-oxo-2-(2-(trifluoromethyl)phenyl)-1,4-dihydroquinolin-7-yl)oxy)acetamido)hexanoic acid (28b)

The procedure for 28b was identical to that described for 28a, except that 6-aminohexanoic acid (88 mg, 0.67 mmol) was used as the starting material. The desired product (28b) was obtained as a beige solid (62 mg, 49% yield).

### Preparation of 7-(2-((3-hydroxy-6-methoxy-4-oxo-2-(2-(trifluoromethyl)phenyl)-1,4-dihydroquinolin-7-yl)oxy)acetamido)heptanoic acid (28c)

The procedure for 28c was identical to that described for 28a, except that 7-aminoheptanoic acid (98 mg, 0.67 mmol) was used as the starting material. The desired product (28c) was obtained as a beige solid (103 mg, 80% yield).

### Preparation of 8-(2-((3-hydroxy-6-methoxy-4-oxo-2-(2-(trifluoromethyl)phenyl)-1,4-dihydroquinolin-7-yl)oxy)acetamido)octanoic acid (28d)

The procedure for 28d was identical to that described for 28a, except that 8-aminooctanoic acid (107 mg, 0.67 mmol) was used as the starting material. The desired product (28d) was obtained as a beige solid (110 mg, 84% yield).

### Preparation of 3-(2-((3-hydroxy-7-methoxy-4-oxo-2-(2-(trifluoromethyl)phenyl)-1,4-dihydroquinolin-6-yl)oxy)acetamido)propanoic acid (28e)

The procedure for 28e was identical to that described for 28a, except that Cpd002 (100 mg, 0.22 mmol) and O-alanine (22 mg, 0.22 mmol) were used as the starting materials. The desired product (28e) was obtained as a beige solid (63 mg, 54% yield).

### Preparation of 4-(2-((3-hydroxy-7-methoxy-4-oxo-2-(2-(trifluoromethyl)phenyl)-1,4-dihydroquinolin-6-yl)oxy)acetamido)butanoic acid (28f)

The procedure for 28f was identical to that described for 28a, except that Cpd002 (100 mg, 0.22 mmol) and γ-aminobutyric acid (25 mg, 0.22 mmol) were used as the starting materials. The desired product (28f) was obtained as a beige solid (64 mg, 54% yield).

### Preparation of 5-(2-((3-hydroxy-7-methoxy-4-oxo-2-(2-(trifluoromethyl)phenyl)-1,4-dihydroquinolin-6-yl)oxy)acetamido)pentanoic acid (28g)

The procedure for 28g was identical to that described for 28a, except that Cpd002 (100 mg, 0.22 mmol) and 5-aminopentanoic acid (53 mg, 0.44 mmol) were used as the starting materials. The desired product (28g) was obtained as a beige solid (39 mg, 32% yield).

### Preparation of 6-(2-((3-hydroxy-7-methoxy-4-oxo-2-(2-(trifluoromethyl)phenyl)-1,4-dihydroquinolin-6-yl)oxy)acetamido)hexanoic acid (28h)

The procedure for 28h was identical to that described for 28a, except that Cpd002 (100 mg, 0.22 mmol) and 6-aminohexanoic acid (32 mg, 0.22 mmol) were used as the starting materials. The desired product (28h) was obtained as a beige solid (76 mg, 61% yield).

### Preparation of 7-(2-((3-hydroxy-7-methoxy-4-oxo-2-(2-(trifluoromethyl)phenyl)-1,4-dihydroquinolin-6-yl)oxy)acetamido)heptanoic acid (28i)

The procedure for 28i was identical to that described for 28a, except that Cpd002 (100 mg, 0.22 mmol) and 7-aminoheptanoic acid (65 mg, 0.44 mmol) were used as the starting materials. The desired product (28i) was obtained as a beige solid (102 mg, 79% yield).

### Preparation of 8-(2-((3-hydroxy-7-methoxy-4-oxo-2-(2-(trifluoromethyl)phenyl)-1,4-dihydroquinolin-6-yl)oxy)acetamido)octanoic acid (28j)

The procedure for 28j was identical to that described for 28a, except that Cpd002 (100 mg, 0.22 mmol) and 8-aminooctanoic acid (39 mg, 0.22 mmol) were used as the starting materials. The desired product (28j) was obtained as a beige solid (86 mg, 65% yield).

### Preparation of N-hydroxy-5-(2-((3-hydroxy-6-methoxy-4-oxo-2-(2-(trifluoromethyl)phenyl)-1,4-dihydroquinolin-7-yl)oxy)acetamido)pentanamide (Cpd009)

A mixture of 5-(2-((3-hydroxy-6-methoxy-4-oxo-2-(2-(trifluoromethyl)phenyl)-1,4-dihydroquinolin-7-yl)oxy)acetamido)pentanoic acid (28a, 82 mg, 0.15 mmol), DIPEA (26 μL, 0.15 mmol) and HATU (63 mg, 0.17 mmol) in 1 mL of DMAC was stirred at room temperature for 30 minutes. Subsequently, a solution of NH₂OTHP (53 mg, 0.45 mmol) in 1 mL of DMAC was added. The mixture was stirred at room temperature for 16 hours. Upon completion, the reaction mixture was poured into 20 mL of cold water. The resulting white precipitate was collected by filtration, rinsed with deionized water, and dried under vacuum. The crude product was purified by flash chromatography using a gradient elution of 0% to 20% MeOH in DCM. This intermediate was then dissolved in 2 mL of 1,4-dioxane and 2 mL of 2 N HCl in dimethyl ether solution was added dropwise at room temperature. The reaction mixture was stirred at room temperature for 1 h. Upon completion, the mixture was concentrated under reduced pressure to afford the desired product (Cpd009) as a brown solid (7 mg, 8% yield). ¹H NMR (500 MHz, DMSO-d6) δ 13.02-12.70 (m, 2H), 10.36 (br s, 1H), 8.91 (br s, 1H), 8.12 (t, J=5.7 Hz, 1H), 7.95 (d, J=7.7 Hz, 1H), 7.85 (t, J=7.3 Hz, 1H), 7.80 (t, J=7.6 Hz, 1H), 7.68 (d, J=7.4 Hz, 1H), 7.58 (s, 1H), 6.98 (s, 1H), 4.61 (d, J=2.7 Hz, 2H), 3.94 (s, 3H), 3.09 (dd, J=12.6, 6.3 Hz, 2H), 1.96-1.86 (m, 2H), 1.51-1.32 (m, 4H).

### Preparation of N-hydroxy-6-(2-((3-hydroxy-6-methoxy-4-oxo-2-(2-(trifluoromethyl)phenyl)-1,4-dihydroquinolin-7-yl)oxy)acetamido)hexanamide (Cpd010)

The procedure for Cpd010 was identical to that described for Cpd009, except that 6-(2-((3-hydroxy-6-methoxy-4-oxo-2-(2-(trifluoromethyl)phenyl)-1,4-dihydroquinolin-7-yl)oxy)acetamido)hexanoic acid (28b, 62 mg, 0.11 mmol) was used as the starting material. The desired product (Cpd010) was obtained as a brown solid (8 mg, 9% yield). ¹H NMR (500 MHz, DMSO-d6) δ 12.96-12.62 (m, 2H), 10.21 (s, 1H), 8.87 (br s, 1H), 8.09 (t, J=5.6 Hz, 1H), 7.95 (d, J=7.8 Hz, 1H), 7.85 (t, J=7.5 Hz, 1H), 7.79 (t, J=7.6 Hz, 1H), 7.66 (d, J=7.4 Hz, 1H), 7.58 (s, 1H), 6.97 (s, 1H), 4.60 (s, 2H), 3.93 (s, 3H), 3.08 (dd, J=12.5, 6.3 Hz, 2H), 2.13 (t, J=7.4 Hz, 1H), 1.93-1.85 (m, 1H), 1.50-1.34 (m, 4H), 1.25-1.13 (m, 2H).

### Preparation of N-hydroxy-7-(2-((3-hydroxy-6-methoxy-4-oxo-2-(2-(trifluoromethyl)phenyl)-1,4-dihydroquinolin-7-yl)oxy)acetamido)heptanamide (Cpd011)

The procedure for Cpd011 was identical to that described for Cpd009, except that 6-(2-((3-hydroxy-6-methoxy-4-oxo-2-(2-(trifluoromethyl)phenyl)-1,4-dihydroquinolin-7-yl)oxy)acetamido)heptanoic acid (28c, 103 mg, 0.18 mmol) was used as the starting material. The desired product (Cpd011) was obtained as a beige solid (43 mg, 41% yield). ¹H NMR (500 MHz, DMSO-d6) δ 12.96-12.32 (m, 2H), 10.34 (br s, 1H), 8.76 (br s, 1H), 8.07 (s, 1H), 7.95 (d, J=7.9 Hz, 1H), 7.85 (t, J=7.5 Hz, 1H), 7.79 (t, J=7.6 Hz, 1H), 7.66 (d, J=6.8 Hz, 1H), 7.56 (s, 1H), 6.96 (s, 1H), 4.60 (s, 2H), 3.93 (s, 3H), 3.08 (dd, J=12.9, 6.7 Hz, 2H), 1.94-1.84 (m, 2H), 1.49-1.33 (m, 4H), 1.26-1.12 (m, 4H).

### Preparation of N-hydroxy-8-(2-((3-hydroxy-6-methoxy-4-oxo-2-(2-(trifluoromethyl)phenyl)-1,4-dihydroquinolin-7-yl)oxy)acetamido)octanamide (Cpd012)

The procedure for Cpd012 was identical to that described for Cpd009, except that 6-(2-((3-hydroxy-6-methoxy-4-oxo-2-(2-(trifluoromethyl)phenyl)-1,4-dihydroquinolin-7-yl)oxy)acetamido)octanoic acid (28d, 110 mg, 0.19 mmol) was used as the starting material. The desired product (Cpd012) was obtained as a beige solid (43 mg, 38% yield). ¹H NMR (500 MHz, DMSO-d6) δ 13.05-12.49 (m, 2H), 10.33 (br s, 1H), 8.80 (br s, 1H), 8.07 (s, 1H), 7.95 (d, J=7.9 Hz, 1H), 7.86 (t, J=7.5 Hz, 1H), 7.80 (t, J=7.4 Hz, 1H), 7.66 (d, J=6.6 Hz, 1H), 7.58 (s, 1H), 6.98 (s, 1H), 4.60 (s, 2H), 3.93 (s, 3H), 3.08 (dd, J=13.0, 6.8 Hz, 2H), 1.89 (t, J=7.1 Hz, 2H), 1.48-1.32 (m, 4H), 1.25-1.11 (m, 6H).

### Preparation of N-hydroxy-3-(2-((3-hydroxy-7-methoxy-4-oxo-2-(2-(trifluoromethyl)phenyl)-1,4-dihydroquinolin-6-yl)oxy)acetamido)propanamide (Cpd013)

The procedure for Cpd013 was identical to that described for Cpd009, except that 6-(2-((3-hydroxy-6-methoxy-4-oxo-2-(2-(trifluoromethyl)phenyl)-1,4-dihydroquinolin-7-yl)oxy)acetamido)propanoic acid (28e, 63 mg, 0.12 mmol) was used as the starting material. The desired product (Cpd013) was obtained as a beige solid (10 mg, 10% yield). ¹H NMR (500 MHz, DMSO-d6) δ 13.25-12.90 (m, 2H), 10.52 (s, 1H), 8.92 (br s, 1H), 8.19 (t, J=5.7 Hz, 1H), 7.97 (d, J=7.7 Hz, 1H), 7.87 (t, J=7.4 Hz, 1H), 7.81 (t, J=7.6 Hz, 1H), 7.68 (d, J=7.4 Hz, 1H), 7.53 (s, 1H), 7.14 (s, 1H), 4.63 (s, 2H), 3.90 (s, 3H), 3.40-3.30 (m, 2H), 2.20 (t, J=7.1 Hz, 2H).

### Preparation of N-hydroxy-4-(2-((3-hydroxy-7-methoxy-4-oxo-2-(2-(trifluoromethyl)phenyl)-1,4-dihydroquinolin-6-yl)oxy)acetamido)butanamide (Cpd014)

The procedure for Cpd014 was identical to that described for Cpd009, except that 6-(2-((3-hydroxy-6-methoxy-4-oxo-2-(2-(trifluoromethyl)phenyl)-1,4-dihydroquinolin-7-yl)oxy)acetamido)butanoic acid (28f, 64 mg, 0.12 mmol) was used as the starting material. The desired product (Cpd014) was obtained as a beige solid (44 mg, 67% yield). ¹H NMR (500 MHz, DMSO-d6) δ 13.51-12.95 (m, 2H), 10.42 (s, 1H), 8.95 (br s, 1H), 8.20 (s, 1H), 7.97 (d, J=7.8 Hz, 1H), 7.88 (t, J=7.5 Hz, 1H), 7.82 (t, J=7.6 Hz, 1H), 7.69 (d, J=7.4 Hz, 1H), 7.54 (s, 1H), 7.16 (s, 1H), 4.65 (s, 2H), 3.91 (s, 3H), 3.20-3.07 (m, 2H), 1.98 (t, J=7.5 Hz, 2H), 1.72-1.62 (m, 2H).

### Preparation of N-hydroxy-4-(2-((3-hydroxy-7-methoxy-4-oxo-2-(2-(trifluoromethyl)phenyl)-1,4-dihydroquinolin-6-yl)oxy)acetamido)pentanamide (Cpd015)

The procedure for Cpd015 was identical to that described for Cpd009, except that 6-(2-((3-hydroxy-6-methoxy-4-oxo-2-(2-(trifluoromethyl)phenyl)-1,4-dihydroquinolin-7-yl)oxy)acetamido)pentanoic acid (28 g, 121 mg, 0.22 mmol) was used as the starting material. The desired product (Cpd015) was obtained as a beige solid (18 mg, 14% yield). ¹H NMR (500 MHz, DMSO-d6) δ 13.10-12.57 (m, 2H), 10.37 (br s, 1H), 8.89 (br s, 1H), 8.12 (s, 1H), 7.96 (d, J=7.8 Hz, 1H), 7.87 (t, J=7.4 Hz, 1H), 7.81 (t, J=7.5 Hz, 1H), 7.67 (d, J=7.3 Hz, 1H), 7.51 (s, 1H), 7.10 (s, 1H), 4.62 (s, 2H), 3.90 (s, 3H), 3.19-3.06 (m, 2H), 1.95 (t, J=7.2 Hz, 2H), 1.56-1.35 (m, 4H).

### Preparation of N-hydroxy-4-(2-((3-hydroxy-7-methoxy-4-oxo-2-(2-(trifluoromethyl)phenyl)-1,4-dihydroquinolin-6-yl)oxy)acetamido)hexanamide (Cpd016)

The procedure for Cpd016 was identical to that described for Cpd009, except that 6-(2-((3-hydroxy-6-methoxy-4-oxo-2-(2-(trifluoromethyl)phenyl)-1,4-dihydroquinolin-7-yl)oxy)acetamido)hexanoic acid (28h, 86 mg, 0.15 mmol) was used as the starting material. The desired product (Cpd016) was obtained as a beige solid (33 mg, 37% yield). ¹H NMR (500 MHz, DMSO-d6) δ 13.14-12.47 (m, 2H), 10.36 (br s, 1H), 8.77 (br s, 1H), 8.10 (s, 1H), 7.96 (d, J=7.9 Hz, 1H), 7.87 (t, J=7.5 Hz, 1H), 7.81 (t, J=7.5 Hz, 1H), 7.67 (d, J=7.1 Hz, 1H), 7.51 (s, 1H), 7.11 (s, 1H), 4.62 (s, 2H), 3.90 (s, 3H), 3.12 (dd, J=12.8, 6.2 Hz, 2H), 1.93 (t, J=7.4 Hz, 2H), 1.52-1.38 (m, 4H), 1.28-1.19 (m, 2H).

### Preparation of N-hydroxy-4-(2-((3-hydroxy-7-methoxy-4-oxo-2-(2-(trifluoromethyl)phenyl)-1,4-dihydroquinolin-6-yl)oxy)acetamido)heptanamide (Cpd017)

The procedure for Cpd017 was identical to that described for Cpd009, except that 6-(2-((3-hydroxy-6-methoxy-4-oxo-2-(2-(trifluoromethyl)phenyl)-1,4-dihydroquinolin-7-yl)oxy)acetamido)heptanoic acid (28i, 102 mg, 0.18 mmol) was used as the starting material. The desired product (Cpd017) was obtained as a beige solid (38 mg, 37% yield). ¹H NMR (500 MHz, DMSO-d6) δ 13.27-12.38 (m, 2H), 10.36 (br s, 1H), 8.94 (br s, 1H), 8.14-8.05 (m, 1H), 7.97 (d, J=7.7 Hz, 1H), 7.87 (t, J=7.5 Hz, 1H), 7.81 (t, J=7.4 Hz, 1H), 7.68 (d, J=7.2 Hz, 1H), 7.55-7.46 (m, 1H), 7.16-7.04 (m, 1H), 4.63 (s, 2H), 3.90 (s, 3H), 3.12 (dd, J=12.9, 6.3 Hz, 2H), 1.92 (t, J=7.3 Hz, 2H), 1.50-1.37 (m, 4H), 1.28-1.18 (m, 4H).

### Preparation of N-hydroxy-4-(2-((3-hydroxy-7-methoxy-4-oxo-2-(2-(trifluoromethyl)phenyl)-1,4-dihydroquinolin-6-yl)oxy)acetamido)octanamide (Cpd018)

The procedure for Cpd018 was identical to that described for Cpd009, except that 6-(2-((3-hydroxy-6-methoxy-4-oxo-2-(2-(trifluoromethyl)phenyl)-1,4-dihydroquinolin-7-yl)oxy)acetamido)octanoic acid (28j, 86 mg, 0.15 mmol) was used as the starting material. The desired product (Cpd018) was obtained as a brown solid (39 mg, 44% yield). ¹H NMR (500 MHz, DMSO-d6) δ 13.40-12.84 (m, 2H), 10.36 (br s, 1H), 8.97 (br s, 1H), 8.14-8.06 (m, 1H), 7.97 (d, J=7.8 Hz, 1H), 7.88 (t, J=7.5 Hz, 1H), 7.82 (t, J=7.5 Hz, 1H), 7.69 (d, J=7.1 Hz, 1H), 7.57-7.49 (m, 1H), 7.18-7.09 (m, 1H), 4.64 (s, 2H), 3.91 (s, 3H), 3.12 (dd, J=13.0, 6.6 Hz, 2H), 1.96-1.88 (m, 2H), 1.50-1.36 (m, 4H), 1.27-1.13 (m, 6H).

### Example 5

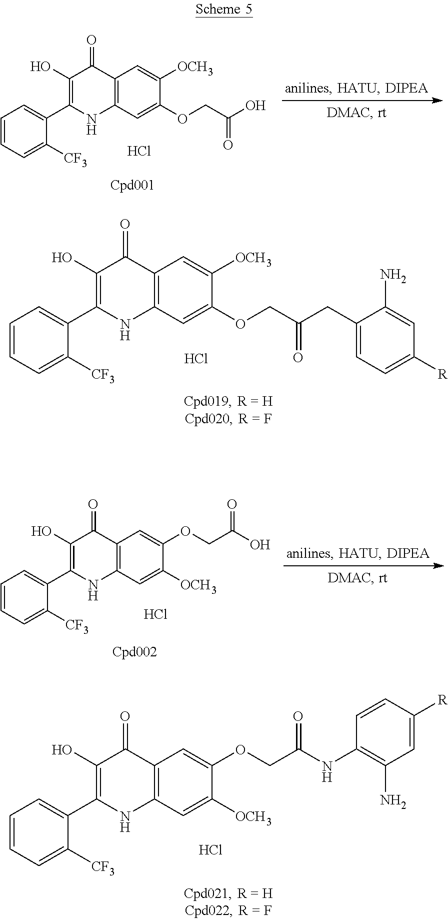

### Preparation of N-(2-aminophenyl)-2-((3-hydroxy-6-methoxy-4-oxo-2-(2-(trifluoromethyl)phenyl)-1,4-dihydroquinolin-7-yl)oxy)acetamide (Cpd019)

To a mixture of 2-((3-hydroxy-6-methoxy-4-oxo-2-(2-(trifluoromethyl)phenyl)-1,4-dihydroquinolin-7-yl)oxy)acetic acid hydrochloride (Cpd001, 100 mg, 0.22 mmol), 1,2-phenylenediamine dihydrochloride (45 mg, 0.25 mmol) in 2 mL of DMAC was added DIPEA (120 μL, 0.68 mmol) and HATU (94 mg, 0.25 mmol) on an ice bath. The mixture was stirred at room temperature for 18 hours. Upon completion, the reaction mixture was poured into 20 mL of cold water. The resulting brown precipitate was collected by filtration, rinsed with deionized water, and dried under vacuum. The crude product was purified by flash chromatography using a gradient elution of 0% to 20% MeOH in DCM. The desired product (Cpd019) was obtained as a brown solid (62 mg, 55% yield). ¹H NMR (500 MHz, DMSO-d6) δ 11.69 (s, 1H), 9.31 (s, 1H), 8.11 (br s, 1H), 7.92 (d, J=7.8 Hz, 1H), 7.82 (t, J=7.5 Hz, 1H), 7.75 (t, J=7.4 Hz, 1H), 7.62 (d, J=7.4 Hz, 1H), 7.49 (s, 1H), 7.19 (d, J=7.8 Hz, 1H), 6.95 (s, 1H), 6.91 (t, J=7.6 Hz, 1H), 6.71 (d, J=7.9 Hz, 1H), 6.53 (t, J=7.5 Hz, 1H), 4.92 (s, 2H), 4.80 (s, 2H), 3.90 (s, 3H).

### Preparation of N-(2-amino-4-fluorophenyl)-2-((3-hydroxy-6-methoxy-4-oxo-2-(2-(trifluoromethyl)phenyl)-1,4-dihydroquinolin-7-yl)oxy)acetamide (Cpd020)

To a mixture of 2-((3-hydroxy-6-methoxy-4-oxo-2-(2-(trifluoromethyl)phenyl)-1,4-dihydroquinolin-7-yl)oxy)acetic acid hydrochloride (Cpd001, 100 mg, 0.22 mmol), 4-fluoro-1,2-phenylenediamine (28 mg, 0.22 mmol) in 2 mL of DMAC was added DIPEA (39 μL, 0.22 mmol) and HATU (94 mg, 0.25 mmol) on an ice bath. The mixture was stirred at room temperature for 18 hours. Upon completion, the reaction mixture was poured into 20 mL of cold water. The resulting brown precipitate was collected by filtration, rinsed with deionized water, and dried under vacuum. The crude product was purified by flash chromatography using a gradient elution of 0% to 20% MeOH in DCM. The desired product (Cpd020) was obtained as a brown solid (91 mg, 78% yield). ¹H NMR (500 MHz, DMSO-d6) δ 11.67 (s, 1H), 9.28 (s, 1H), 8.10 (br s, 1H), 7.92 (d, J=7.8 Hz, 1H), 7.83 (t, J=7.5 Hz, 1H), 7.76 (t, J=7.6 Hz, 1H), 7.62 (d, J=7.5 Hz, 1H), 7.49 (s, 1H), 7.09 (dd, J=8.6, 6.4 Hz, 1H), 6.94 (s, 1H), 6.47 (dd, J=11.2, 2.8 Hz, 1H), 6.29 (td, J=8.5, 2.8 Hz, 1H), 5.26 (s, 2H), 4.79 (d, J=1.8 Hz, 2H), 3.90 (s, 3H).

### Preparation of N-(2-aminophenyl)-2-((3-hydroxy-7-methoxy-4-oxo-2-(2-(trifluoromethyl)phenyl)-1,4-dihydroquinolin-6-yl)oxy)acetamide (Cpd021)

The procedure for Cpd021 was identical to that described for Cpd019, except that Cpd002 (100 mg, 0.22 mmol) was used as the starting material. The desired product (Cpd021) was obtained as a brown solid (28 mg, 31% yield). ¹H NMR (500 MHz, DMSO-d6) δ 11.63 (s, 1H), 9.38 (s, 1H), 8.10 (br s, 1H), 7.93 (d, J=7.9 Hz, 1H), 7.83 (t, J=7.6 Hz, 1H), 7.76 (t, J=7.6 Hz, 1H), 7.61 (d, J=7.5 Hz, 1H), 7.56 (s, 1H), 7.20 (d, J=7.6 Hz, 1H), 6.97 (s, 1H), 6.93 (t, J=7.6 Hz, 1H), 6.74 (d, J=8.0 Hz, 1H), 6.55 (t, J=7.5 Hz, 1H), 4.94 (s, 2H), 4.82 (s, 2H), 3.86 (s, 3H).

### Preparation of N-(2-amino-4-fluorophenyl)-2-((3-hydroxy-7-methoxy-4-oxo-2-(2-(trifluoromethyl)phenyl)-1,4-dihydroquinolin-6-yl)oxy)acetamide (Cpd022)

The procedure for Cpd022 was identical to that described for Cpd020, except that Cpd002 (100 mg, 0.22 mmol) was used as the starting material. The crude product was washed with MeOH to obtain the desired product (Cpd022) as a beige solid (47 mg, 51% yield). ¹H NMR (500 MHz, DMSO-d6) δ 11.64 (s, 1H), 9.36 (s, 1H), 8.11 (br s, 1H), 7.93 (d, J=7.9 Hz, 1H), 7.83 (t, J=7.5 Hz, 1H), 7.76 (t, J=7.7 Hz, 1H), 7.61 (d, J=7.5 Hz, 1H), 7.55 (s, 1H), 7.11 (dd, J=8.6, 6.3 Hz, 1H), 6.97 (s, 1H), 6.50 (dd, J=11.2, 2.9 Hz, 1H), 6.32 (td, J=8.5, 2.9 Hz, 1H), 5.28 (s, 2H), 4.80 (s, 2H), 3.86 (s, 3H).

### Example 6

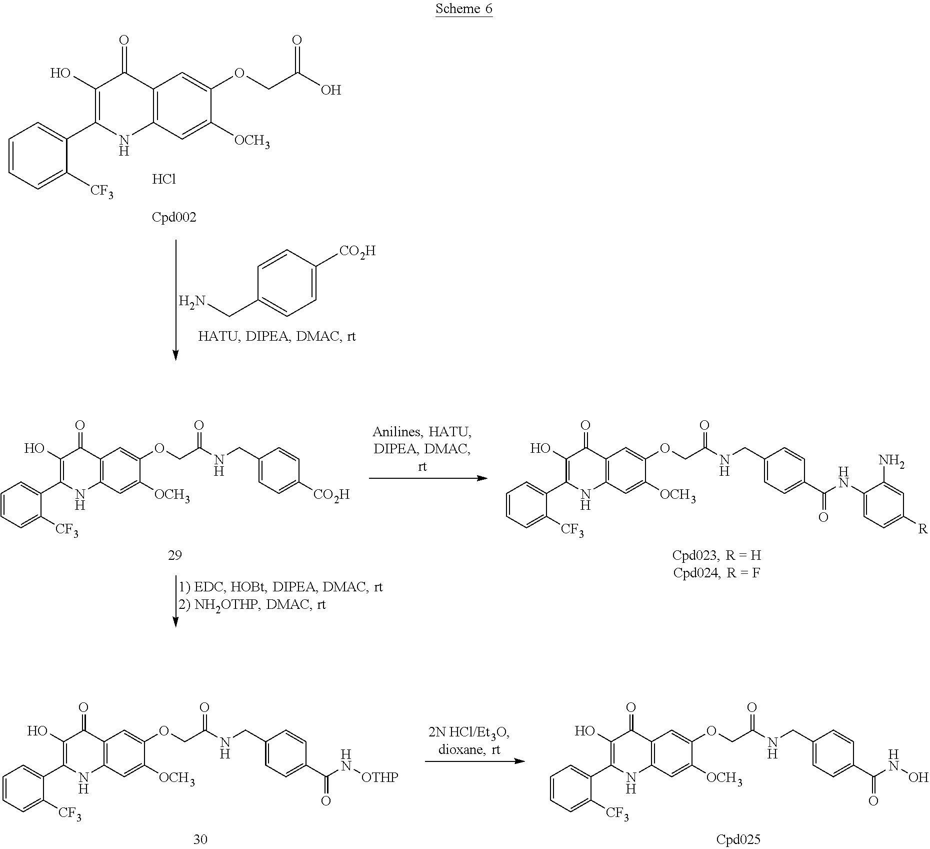

### 4-((2-((3-hydroxy-7-methoxy-4-oxo-2-(2-(trifluoromethyl)phenyl)-1,4-dihydroquinolin-6-yl)oxy)acetamido)methyl)benzoic acid (29)

To a solution of Cpd002 (200 mg, 0.45 mmol) in 2 mL of DMAC was added DIPEA (76 μL, 0.45 mmol) and HATU (188 mg, 0.49 mmol) at room temperature. This solution (solution A) was stirred at room temperature for 45 mins. Separately, a mixture of 4-(aminomethyl)benzoic acid (102 mg, 0.67 mmol) and DIPEA (116 μL, 0.67 mmol) in 2 mL of DMAC was stirred at room temperature for 45 mins (solution B). Solution A was added to solution B and the resulting mixture was stirred at room temperature for 16 h. Upon completion, the reaction mixture was poured into 20 mL of cold water and acidified with 2N HCl aqueous solution to pH=1. The resulting white precipitate was collected by filtration, rinsed with deionized water, and dried under vacuum to afford the desired product (29) as a white solid (170 mg, 65% yield).

### Preparation of N-(2-aminophenyl)-4-((2-((3-hydroxy-7-methoxy-4-oxo-2-(2-(trifluoromethyl)phenyl)-1,4-dihydroquinolin-6-yl)oxy)acetamido)methyl)benzamide (Cpd023)

The procedure for Cpd023 was identical to that described for Cpd019, except that compound 29 (85 mg, 0.15 mmol) was used as the starting material. The crude product was washed with MeOH to obtain the desired product (Cpd023) as a beige solid (44 mg, 47% yield). ¹H NMR (500 MHz, DMSO-d6) δ 11.64 (s, 1H), 9.64 (s, 1H), 8.72 (t, J=6.0 Hz, 1H), 8.11 (br s, 1H), 7.95-7.90 (m, 3H), 7.82 (t, J=7.4 Hz, 1H), 7.76 (t, J=7.6 Hz, 1H), 7.61 (d, J=7.3 Hz, 1H), 7.52 (s, 1H), 7.42 (d, J=8.0 Hz, 2H), 7.16 (d, J=7.7 Hz, 1H), 6.98-6.95 (m, 2H), 6.79 (d, J=8.0 Hz, 1H), 6.60 (t, J=7.4 Hz, 1H), 4.96 (br s, 2H), 4.70 (s, 2H), 4.49-4.37 (m, 2H), 3.85 (s, 3H).

### Preparation of N-(2-amino-4-fluorophenyl)-4-((2-((3-hydroxy-7-methoxy-4-oxo-2-(2-(trifluoromethyl)phenyl)-1,4-dihydroquinolin-6-yl)oxy)acetamido)methyl)benzamide (Cpd024)

The procedure for Cpd024 was identical to that described for Cpd020, except that compound 29 (85 mg, 0.15 mmol) was used as the starting material. The crude product was washed with MeOH to obtain the desired product (Cpd024) was obtained as a beige solid (61 mg, 64% yield). ¹H NMR (500 MHz, DMSO-d6) δ 11.63 (s, 1H), 9.56 (s, 1H), 8.71 (t, J=6.0 Hz, 1H), 8.10 (br s, 1H), 7.95-7.88 (m, 3H), 7.82 (t, J=7.4 Hz, 1H), 7.76 (t, J=7.7 Hz, 1H), 7.61 (d, J=7.5 Hz, 1H), 7.52 (s, 1H), 7.41 (d, J=8.1 Hz, 2H), 7.10 (dd, J=8.5, 6.4 Hz, 1H), 6.96 (s, 1H), 6.53 (dd, J=11.2, 2.9 Hz, 1H), 6.35 (td, J=8.5, 2.8 Hz, 1H), 5.23 (s, 2H), 4.69 (s, 2H), 4.50-4.38 (m, 2H), 3.85 (s, 3H).

### Preparation of 4-((2-((3-hydroxy-7-methoxy-4-oxo-2-(2-(trifluoromethyl)phenyl)-1,4-dihydroquinolin-6-yl)oxy)acetamido)methyl)-N-((tetrahydro-2H-pyran-2-yl)oxy)benzamide (30)

A mixture of 29 (84 mg, 0.15 mmol), EDC (1-ethyl-3-(3-dimethylaminopropyl)carbodiimide hydrochloride) (31 mg, 0.16 mmol), HOBt (27 mg, 0.16 mmol) and DIPEA (25 μL, 0.15 mmol) in 1 mL of DMAC was stirred at room temperature for 30 mins. The reaction solution was added to a solution of NH₂OTHP (19 mg, 0.16 mmol) in 1 mL of DMAC. The resulting mixture was stirred at room temperature for 16 h. Upon completion, the reaction mixture was poured into 20 mL of cold water. The resulting beige precipitate was collected by filtration, rinsed with deionized water, and dried under vacuum. The crude product was purified by flash chromatography using a gradient elution of 0% to 25% MeOH in DCM. The desired product (30) was obtained as a beige solid (26 mg, 28% yield).

### Preparation of N-hydroxy-4-((2-((3-hydroxy-7-methoxy-4-oxo-2-(2-(trifluoromethyl)phenyl)-1,4-dihydroquinolin-6-yl)oxy)acetamido)methyl)benzamide hydrochloride (Cpd025)

To a stirred solution of 30 (26 mg, 0.04 mmol) in 1 mL of 1,4-dioxane was added 2 mL of 2 N HCl in dimethyl ether solution dropwise at room temperature. The reaction mixture was stirred at room temperature for 3 h. Upon completion, the mixture was concentrated under reduced pressure and the residue was washed with EtOAc to afford the desired product (Cpd025) as a white solid (24 mg, 99% yield). ¹H NMR (500 MHz, DMSO-d6) δ 12.28-12.08 (m, 2H), 11.18 (s, 1H), 8.69 (t, J=6.0 Hz, 1H), 8.47 (br s, 1H), 7.95 (d, J=7.9 Hz, 1H), 7.85 (t, J=7.5 Hz, 1H), 7.79 (t, J=7.7 Hz, 1H), 7.70 (d, J=8.1 Hz, 2H), 7.65 (d, J=7.7 Hz, 1H), 7.53 (s, 1H), 7.36 (d, J=8.1 Hz, 2H), 7.02 (s, 1H), 4.71 (s, 2H), 4.40 (d, J=3.1 Hz, 2H), 3.87 (s, 3H).

### Example 7

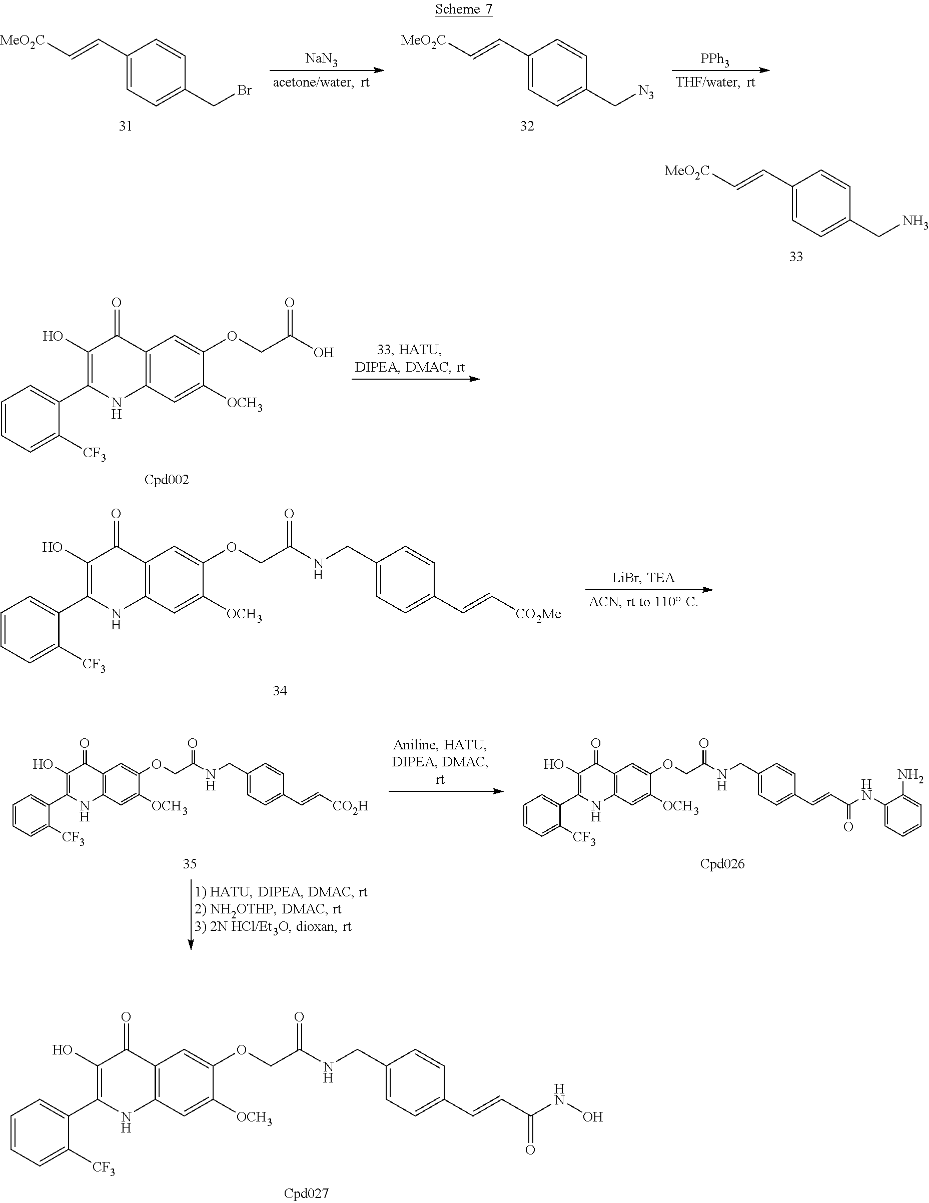

### Preparation of methyl 3-(4-(azidomethyl)phenyl)acrylate (32)

To a mixture of methyl 3-(4-bromomethyl) cinnamate (31, 1 g, 3.92 mmol) and sodium azide (306 mg, 4.70 mmol) in 20 mL of 5:1 acetone/water was stirred at room temperature for 18 h. The reaction mixture was diluted with brine and extracted with EtOAc. The organic layer was dried over anhydrous MgSO₄, filtered, and concentrated under reduced pressure. The crude product was purified by flash chromatography using a gradient elution of 0% to 10% EtOAc in hexanes. The desired product (32) was obtained as a clear crystal (785 mg, 92% yield).

### Preparation of methyl 3-(4-(aminomethyl)phenyl)acrylate (33)

Compound 32 (785 mg, 3.61 mmol) was dissolved in 8 mL of THF and cooled on an ice bath. Triphenylphosphine (1.4 g, 5.42 mmol) was added portionwise to the solution. The reaction mixture was stirred at rt for 3 h until gas generation subsided. To the reaction mixture, 3 mL of water was added and stirred at room temperature for 15 h. The reaction mixture was concentrated to remove the majority of THF. The residue was dissolved in 1N HCl aqueous solution and washed with EtOAc. The aqueous layer was adjusted to pH 10 with 1N NaOH aqueous solution and further extracted with EtOAc. The combined organic layers were washed with brine, dried over anhydrous MgSO₄, filtered, and concentrated under reduced pressure. The crude product was purified by flash chromatography using an isocratic elution of 2:1 EtOAc/MeOH. The desired product (33) was obtained as a pale yellow solid (178 mg, 26% yield).

### Preparation of methyl 3-(4-((2-((3-hydroxy-7-methoxy-4-oxo-2-(2-(trifluoromethyl)phenyl)-1,4-dihydroquinolin-6-yl)oxy)acetamido)methyl)phenyl)acrylate (34)

A mixture of Cpd002 (400 mg, 0.90 mmol), DIPEA (153 μL, 0.90 mmol) and HATU (375 mg, 0.99 mmol) in 6 mL of DMAC was stirred at room temperature for 30 minutes. Subsequently, compound 33 (178 mg, 0.90 mmol) was added to the reaction and the mixture was stirred at room temperature for 16 h. Upon completion, the reaction mixture was poured into 60 mL of cold water. The resulting precipitate was collected by filtration, rinsed with deionized water, and dried under vacuum. The desired product (34) was obtained as a white solid (516 mg, 99% yield).

### Preparation of 3-(4-((2-((3-hydroxy-7-methoxy-4-oxo-2-(2-(trifluoromethyl)phenyl)-1,4-dihydroquinolin-6-yl)oxy)acetamido)methyl)phenyl)acrylic acid (35)

A mixture of 34 (260 mg, 0.45 mmol), LiBr (776 mg, 8.93 mmol) and lithium bromide (187 μL, 1.34 mmol) in 3 mL of wet ACN containing 60 μL of water was stirred at 110° C. for 24 h in a sealed tube. After cooling to room temperature, the solvent was removed under reduced pressure and the residue was suspended in water. The suspension was acidified to pH 1 using 2N HCl aqueous solution. The resulting precipitate was collected by filtration, rinsed with deionized water, and dried under vacuum. The crude product was purified by flash chromatography using a gradient elution of 2% to 30% MeOH in DCM. The desired product (35) was obtained as a brown solid (63 mg, 25% yield).

### Preparation of (E)-N-(2-aminophenyl)-3-(4-((2-((3-hydroxy-7-methoxy-4-oxo-2-(2-(trifluoromethyl)phenyl)-1,4-dihydroquinolin-6-yl)oxy)acetamido)methyl)phenyl)acrylamide (Cpd026)

The procedure for Cpd026 was identical to that described for Cpd019, except that compound 35 (19 mg, 0.03 mmol) was used as the starting material. The crude product was purified by flash chromatography using a gradient elution of 0% to 25% MeOH in DCM. The desired product (Cpd026) was obtained as a brown solid (3 mg, 14% yield). ¹H NMR (500 MHz, DMSO-d6) δ 11.63 (s, 1H), 9.36 (s, 1H), 8.66 (d, J=6.0 Hz, 1H), 8.10 (br s, 1H), 7.93 (d, J=7.9 Hz, 1H), 7.86-7.79 (m, 1H), 7.79-7.72 (m, 1H), 7.61 (d, J=7.5 Hz, 1H), 7.56 (d, J=8.0 Hz, 2H), 7.52 (t, J=7.8 Hz, 2H), 7.39-7.30 (m, 3H), 6.96 (s, 1H), 6.94-6.84 (m, 2H), 6.74 (d, J=7.9 Hz, 1H), 6.57 (t, J=7.6 Hz, 1H), 4.95 (s, 2H), 4.69 (s, 2H), 4.39 (d, J=5.9 Hz, 2H), 3.85 (s, 3H).

### Preparation of (E)-N-hydroxy-3-(4-((2-((3-hydroxy-7-methoxy-4-oxo-2-(2-(trifluoromethyl)phenyl)-1,4-dihydroquinolin-6-yl)oxy)acetamido)methyl)phenyl)acrylamide hydrochloride (Cpd027)

A mixture of compound 35 (63 mg, 0.11 mmol), DIPEA (20 μL, 0.11 mmol) and HATU (46 mg, 0.12 mmol) in 1 mL of DMAC was stirred at room temperature for 30 minutes. Subsequently, a solution of NH₂OTHP (39 mg, 0.33 mmol) in 1 mL of DMAC was added to the reaction and the mixture was stirred at room temperature for 16 h. Upon completion, the reaction mixture was poured into 20 mL of cold water. The resulting precipitate was collected by filtration, rinsed with deionized water, and dried under vacuum. The crude product was purified by flash chromatography using a gradient elution of 0% to 25% MeOH in DCM. This intermediate was then dissolved in 2 mL of 1,4-dioxane and 2 mL of 2 N HCl in dimethyl ether solution was added dropwise at room temperature. The reaction mixture was stirred at room temperature for 1 h. Upon completion, the mixture was concentrated under reduced pressure to afford the desired product (Cpd027) as a brown solid (20 mg, 29% yield). ¹H NMR (500 MHz, DMSO-d6) δ 12.70-12.22 (m, 2H), 10.75 (br s, 1H), 8.68 (t, J=5.5 Hz, 1H), 7.96 (d, J=7.9 Hz, 1H), 7.86 (t, J=7.5 Hz, 1H), 7.80 (t, J=7.7 Hz, 1H), 7.66 (d, J=7.5 Hz, 1H), 7.54 (s, 1H), 7.50 (d, J=8.1 Hz, 2H), 7.42 (d, J=15.8 Hz, 1H), 7.32 (d, J=8.0 Hz, 2H), 7.05 (s, 1H), 6.44 (d, J=15.8 Hz, 1H), 4.73 (s, 2H), 4.37 (d, J=5.8 Hz, 2H), 3.87 (s, 3H).

### Example 8

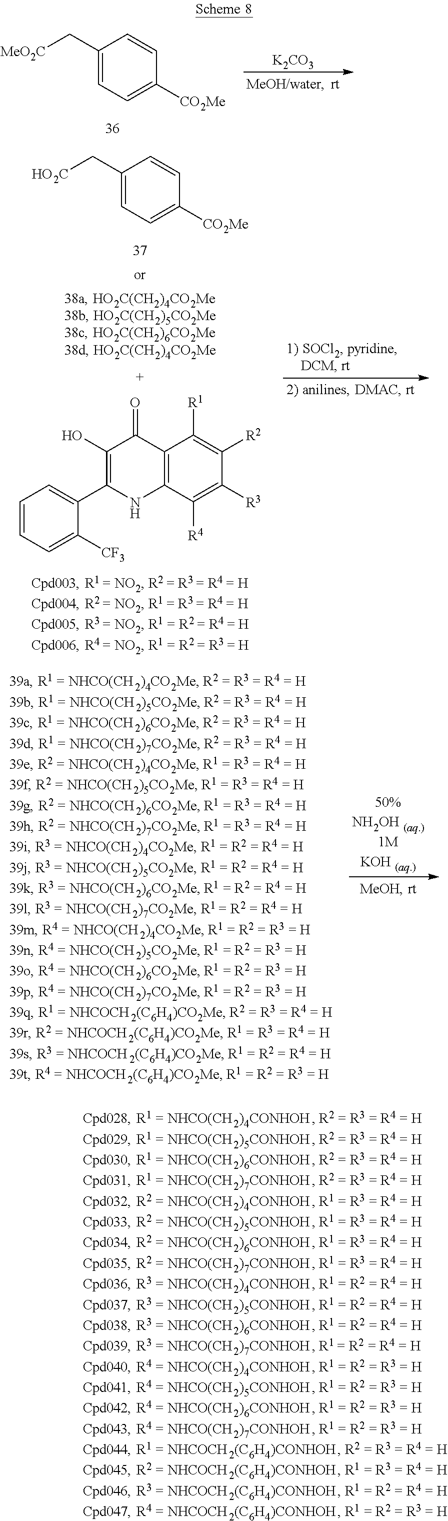

### Preparation of 2-(4-(methoxycarbonyl)phenyl)acetic acid (37)

A mixture of compound 36 (800 mg, 3.84 mmol) and potassium carbonate (929 mg, 5.76 mmol) in 20 mL of 1:1 MeOH/water was stirred at room temperature for 16 h. Upon completion, the reaction mixture was concentrated to 5 mL and diluted with 20 mL of water. The aqueous layer was washed with DCM and acidified to pH 2 with 12M HCl aqueous solution. The aqueous layer was then extracted with EtOAc. The organic layer was dried over anhydrous MgSO₄, filtered, and concentrated under reduced pressure. The desired product (37) was obtained as a white solid (675 mg, 90% yield).

### General Procedure for the Preparation of Compounds 39a-t

To a mixture of carboxylic acid 37 or 38a-d (1.5 equiv.) and pyridine (1.5 equiv.) in DCM (2.0 M) was added SOCl₂ (1.5 equiv.) dropwise on an ice bath. The reaction solution was stirred at room temperature for 30 minutes. Subsequently, a solution of anilines (1.0 equiv.) in DMAC (0.4 M) was added to the reaction on an ice bath and the mixture was stirred at room temperature for 4 h. Upon completion, DCM was evaporated under reduced pressure and the residue was poured into cold water. The resulting precipitate was collected by filtration, rinsed with deionized water, and dried under vacuum. The crude product was purified by flash chromatography using a gradient elution of 1% to 10% MeOH in DCM. The desired product (39a-t) was obtained as a beige to brown solids, often mixed with di-substituted products.

### Preparation of methyl 6-((3-hydroxy-4-oxo-2-(2-(trifluoromethyl)phenyl)-1,4-dihydroquinolin-5-yl)amino)-6-oxohexanoate (39a)

Compound 39a was prepared following the general procedure described above using Cpd003 (150 mg, 0.47 mmol) and 38a (113 mg, 0.70 mmol) as starting materials. The desired product (39a) was obtained as a brown solid (124 mg). ESI-MS: 463.6 [M+H]⁺.

### Preparation of methyl 7-((3-hydroxy-4-oxo-2-(2-(trifluoromethyl)phenyl)-1,4-dihydroquinolin-5-yl)amino)-7-oxoheptanoate (39b)

Compound 39b was prepared following the general procedure described above using Cpd003 (150 mg, 0.47 mmol) and 38b (122 mg, 0.70 mmol) as starting materials. The desired product (39b) was obtained as a brown solid (137 mg). ESI-MS: 477.4 [M+H]⁺.

### Preparation of methyl 8-((3-hydroxy-4-oxo-2-(2-(trifluoromethyl)phenyl)-1,4-dihydroquinolin-5-yl)amino)-8-oxooctanoate (39c)

Compound 39c was prepared following the general procedure described above using Cpd003 (200 mg, 0.62 mmol) and 38c (235 mg, 1.24 mmol) as starting materials. The desired product (39c) was obtained as a brown solid (109 mg). ESI-MS: 491.5 [M+H]⁺.

### Preparation of methyl 9-((3-hydroxy-4-oxo-2-(2-(trifluoromethyl)phenyl)-1,4-dihydroquinolin-5-yl)amino)-9-oxononanoate (39d)

Compound 39d was prepared following the general procedure described above using Cpd003 (150 mg, 0.47 mmol) and 38d (142 mg, 0.70 mmol) as starting materials. The desired product (39d) was obtained as a brown solid (145 mg). ESI-MS: 505.3 [M+H]⁺.

### Preparation of methyl 6-((3-hydroxy-4-oxo-2-(2-(trifluoromethyl)phenyl)-1,4-dihydroquinolin-6-yl)amino)-6-oxohexanoate (39e)

Compound 39e was prepared following the general procedure described above using Cpd004 (150 mg, 0.47 mmol) and 38a (113 mg, 0.70 mmol) as starting materials. The desired product (39e) was obtained as a brown solid (131 mg). ESI-MS: 463.0 [M+H]⁺.

### Preparation of methyl 7-((3-hydroxy-4-oxo-2-(2-(trifluoromethyl)phenyl)-1,4-dihydroquinolin-6-yl)amino)-7-oxoheptanoate (39f)

Compound 39f was prepared following the general procedure described above using Cpd004 (150 mg, 0.47 mmol) and 38b (122 mg, 0.70 mmol) as starting materials. The desired product (39f) was obtained as a brown solid (123 mg). ESI-MS: 477.1 [M+H]⁺.

### Preparation of methyl 8-((3-hydroxy-4-oxo-2-(2-(trifluoromethyl)phenyl)-1,4-dihydroquinolin-6-yl)amino)-8-oxooctanoate (39g)

Compound 39g was prepared following the general procedure described above using Cpd004 (150 mg, 0.47 mmol) and 38c (132 mg, 0.70 mmol) as starting materials. The desired product (39g) was obtained as a beige solid (134 mg). ESI-MS: 491.2 [M+H]⁺.

### Preparation of methyl 9-((3-hydroxy-4-oxo-2-(2-(trifluoromethyl)phenyl)-1,4-dihydroquinolin-6-yl)amino)-9-oxononanoate (39h)

Compound 39h was prepared following the general procedure described above using Cpd004 (150 mg, 0.47 mmol) and 38d (142 mg, 0.70 mmol) as starting materials. The desired product (39h) was obtained as a brown solid (195 mg). ESI-MS: 505.1 [M+H]⁺.

### Preparation of methyl 6-((3-hydroxy-4-oxo-2-(2-(trifluoromethyl)phenyl)-1,4-dihydroquinolin-7-yl)amino)-6-oxohexanoate (39i)

Compound 39i was prepared following the general procedure described above using Cpd005 (150 mg, 0.47 mmol) and 38a (113 mg, 0.70 mmol) as starting materials. The desired product (39i) was obtained as a brown solid (147 mg). ESI-MS: 463.0 [M+H]⁺.

### Preparation of methyl 7-((3-hydroxy-4-oxo-2-(2-(trifluoromethyl)phenyl)-1,4-dihydroquinolin-7-yl)amino)-7-oxoheptanoate (39j)

Compound 39j was prepared following the general procedure described above using Cpd005 (1.0 g, 3.12 mmol) and 38b (707 mg, 4.06 mmol) as starting materials. The desired product (39j) was obtained as a brown solid (1.2 g). ESI-MS: 477.1 [M+H]⁺.

### Preparation of methyl 8-((3-hydroxy-4-oxo-2-(2-(trifluoromethyl)phenyl)-1,4-dihydroquinolin-7-yl)amino)-8-oxooctanoate (39k)

Compound 39k was prepared following the general procedure described above using Cpd005 (150 mg, 0.47 mmol) and 38c (132 mg, 0.70 mmol) as starting materials. The desired product (39k) was obtained as a brown solid (50 mg). ESI-MS: 491.0 [M+H]⁺.

### Preparation of methyl 9-((3-hydroxy-4-oxo-2-(2-(trifluoromethyl)phenyl)-1,4-dihydroquinolin-7-yl)amino)-9-oxononanoate (39l)

Compound 39l was prepared following the general procedure described above using Cpd005 (150 mg, 0.47 mmol) and 38d (142 mg, 0.70 mmol) as starting materials. The desired product (39l) was obtained as a brown solid (185 mg). ESI-MS: 505.0 [M+H]⁺.

### Preparation of methyl 6-((3-hydroxy-4-oxo-2-(2-(trifluoromethyl)phenyl)-1,4-dihydroquinolin-8-yl)amino)-6-oxohexanoate (39m)

Compound 39m was prepared following the general procedure described above using Cpd006 (150 mg, 0.47 mmol) and 38a (113 mg, 0.70 mmol) as starting materials. The desired product (39m) was obtained as a brown solid (179 mg). ESI-MS: 463.0 [M+H]⁺.

### Preparation of methyl 7-((3-hydroxy-4-oxo-2-(2-(trifluoromethyl)phenyl)-1,4-dihydroquinolin-8-yl)amino)-7-oxoheptanoate (39n)

Compound 39n was prepared following the general procedure described above using Cpd006 (150 mg, 0.47 mmol) and 38b (122 mg, 0.70 mmol) as starting materials. The desired product (39n) was obtained as a brown solid (170 mg). ESI-MS: 477.1 [M+H]⁺.

### Preparation of methyl 8-((3-hydroxy-4-oxo-2-(2-(trifluoromethyl)phenyl)-1,4-dihydroquinolin-8-yl)amino)-8-oxooctanoate (39o)

Compound 39o was prepared following the general procedure described above using Cpd006 (200 mg, 0.62 mmol) and 38c (118 mg, 0.62 mmol) as starting materials. The desired product (39o) was obtained as a brown solid (83 mg). ESI-MS: 491.2 [M+H]⁺.

### Preparation of methyl 9-((3-hydroxy-4-oxo-2-(2-(trifluoromethyl)phenyl)-1,4-dihydroquinolin-8-yl)amino)-9-oxononanoate (39p)

Compound 39p was prepared following the general procedure described above using Cpd006 (150 mg, 0.47 mmol) and 38d (142 mg, 0.70 mmol) as starting materials. The desired product (39p) was obtained as a brown solid (176 mg). ESI-MS: 505.1 [M+H]⁺.

### Preparation of methyl 4-(2-((3-hydroxy-4-oxo-2-(2-(trifluoromethyl)phenyl)-1,4-dihydroquinolin-5-yl)amino)-2-oxoethyl)benzoate (39q)

Compound 39q was prepared following the general procedure described above using Cpd003 (100 mg, 0.31 mmol) and 37 (91 mg, 0.47 mmol) as starting materials. The desired product (39q) was obtained as a brown solid (119 mg). ESI-MS: 497.5 [M+H]⁺.

### Preparation of methyl 4-(2-((3-hydroxy-4-oxo-2-(2-(trifluoromethyl)phenyl)-1,4-dihydroquinolin-6-yl)amino)-2-oxoethyl)benzoate (39r)

Compound 39r was prepared following the general procedure described above using Cpd004 (100 mg, 0.31 mmol) and 37 (91 mg, 0.47 mmol) as starting materials. The desired product (39r) was obtained as a grey solid (144 mg). ESI-MS: 497.0 [M+H]⁺.

### Preparation of methyl 4-(2-((3-hydroxy-4-oxo-2-(2-(trifluoromethyl)phenyl)-1,4-dihydroquinolin-7-yl)amino)-2-oxoethyl)benzoate (39s)

Compound 39s was prepared following the general procedure described above using Cpd005 (100 mg, 0.31 mmol) and 37 (91 mg, 0.47 mmol) as starting materials. The desired product (39s) was obtained as a grey solid (130 mg). ESI-MS: 497.0 [M+H]⁺.

### Preparation of methyl 4-(2-((3-hydroxy-4-oxo-2-(2-(trifluoromethyl)phenyl)-1,4-dihydroquinolin-8-yl)amino)-2-oxoethyl)benzoate (39t)

Compound 39t was prepared following the general procedure described above using Cpd006 (100 mg, 0.31 mmol) and 37 (91 mg, 0.47 mmol) as starting materials. The desired product (39t) was obtained as a grey solid (160 mg). ESI-MS: 497.0 [M+H]⁺.

General procedure for the preparation of hydroxamic acids from esters

To a suspension of methyl esters (100 mg) and 50% NH₂OH aqueous solution (1 mL) in MeOH (1 mL) was added 1M KOH aqueous solution (1 mL) dropwise on an ice bath. The reaction solution was stirred at rt for 2-5 h. Upon completion, the solvent was filtered through a 0.22 μm membrane and MeOH and NH₂OH were removed under reduced pressure. The aqueous solution was cooled on ice and acidified to pH 6 with 2N HCl aqueous solution. The precipitate was collected by filtration, rinsed with deionized water, and dried under vacuum. The desired product was obtained as a beige to brown solids.

### Preparation of N¹-hydroxy-N⁶-(3-hydroxy-4-oxo-2-(2-(trifluoromethyl)phenyl)-1,4-dihydroquinolin-5-yl)adipamide (Cpd028)

Cpd028 was prepared following the general procedure described above using 39a (124 mg) as starting material. The desired product (Cpd028) was obtained as a brown solid (91 mg, 42% yield over 2 steps). ¹H NMR (500 MHz, DMSO-d6) δ 13.71 (s, 1H), 12.26 (s, 1H), 10.38 (s, 1H), 8.70 (s, 1H), 8.63 (s, 1H), 8.33 (d, J=7.9 Hz, 1H), 7.96 (d, J=7.9 Hz, 1H), 7.86 (t, J=7.5 Hz, 1H), 7.79 (t, J=7.7 Hz, 1H), 7.67 (d, J=7.5 Hz, 1H), 7.49 (t, J=8.2 Hz, 1H), 7.19 (d, J=8.5 Hz, 1H), 2.41 (t, J=7.3 Hz, 2H), 1.99 (t, J=7.2 Hz, 2H), 1.73-1.61 (m, 2H), 1.61-1.51 (m, 2H). ESI-MS: 464.6 [M+H]⁺.

### Preparation of N¹-hydroxy-N⁷-(3-hydroxy-4-oxo-2-(2-(trifluoromethyl)phenyl)-1,4-dihydroquinolin-5-yl)heptanediamide (Cpd029)

Cpd029 was prepared following the general procedure described above using 39b (137 mg) as starting material. The desired product (Cpd029) was obtained as a brown solid (124 mg, 55% yield over 2 steps). ¹H NMR (500 MHz, DMSO-d6) δ 13.71 (s, 1H), 12.25 (s, 1H), 10.35 (s, 1H), 8.68 (s, 1H), 8.64 (s, 1H), 8.33 (d, J=7.9 Hz, 1H), 7.95 (d, J=7.8 Hz, 1H), 7.86 (t, J=7.5 Hz, 1H), 7.79 (t, J=7.7 Hz, 1H), 7.67 (d, J=7.5 Hz, 1H), 7.49 (t, J=8.2 Hz, 1H), 7.19 (d, J=8.5 Hz, 1H), 2.39 (t, J=7.5 Hz, 2H), 1.95 (t, J=7.3 Hz, 2H), 1.75-1.62 (m, 2H), 1.59-1.47 (m, 2H), 1.39-1.25 (m, 2H). ESI-MS: 478.5 [M+H]⁺.

### Preparation of N¹-hydroxy-N⁸-(3-hydroxy-4-oxo-2-(2-(trifluoromethyl)phenyl)-1,4-dihydroquinolin-5-yl)octanediamide (Cpd030)

Cpd030 was prepared following the general procedure described above using 39c (109 mg) as starting material. The desired product (Cpd030) was obtained as a brown solid (74 mg, 24% yield over 2 steps). ¹H NMR (500 MHz, DMSO-d6) δ 13.70 (s, 1H), 12.25 (s, 1H), 10.34 (s, 1H), 8.68 (s, 1H), 8.62 (s, 1H), 8.33 (d, J=7.9 Hz, 1H), 7.95 (d, J=7.8 Hz, 1H), 7.86 (t, J=7.5 Hz, 1H), 7.79 (t, J=7.7 Hz, 1H), 7.67 (d, J=7.5 Hz, 1H), 7.49 (t, J=8.2 Hz, 1H), 7.19 (d, J=8.5 Hz, 1H), 2.39 (t, J=7.5 Hz, 2H), 1.94 (t, J=7.3 Hz, 2H), 1.74-1.62 (m, 2H), 1.55-1.44 (m, 2H), 1.39-1.20 (m, 4H). ESI-MS: 492.2 [M+H]⁺.

### Preparation of N¹-hydroxy-N⁹-(3-hydroxy-4-oxo-2-(2-(trifluoromethyl)phenyl)-1,4-dihydroquinolin-5-yl)nonanediamide (Cpd031)

Cpd031 was prepared following the general procedure described above using 39d (145 mg) as starting material. The desired product (Cpd031) was obtained as a brown solid (121 mg, 51% yield over 2 steps). ¹H NMR (500 MHz, DMSO-d6) δ 13.70 (s, 1H), 12.25 (s, 1H), 10.33 (s, 1H), 8.67 (s, 1H), 8.61 (s, 1H), 8.33 (d, J=7.9 Hz, 1H), 7.95 (d, J=7.9 Hz, 1H), 7.86 (t, J=7.5 Hz, 1H), 7.79 (t, J=7.7 Hz, 1H), 7.67 (d, J=7.5 Hz, 1H), 7.49 (t, J=8.2 Hz, 1H), 7.19 (d, J=8.5 Hz, 1H), 2.39 (t, J=7.5 Hz, 2H), 1.93 (t, J=7.3 Hz, 2H), 1.73-1.63 (m, 2H), 1.52-1.43 (m, 2H), 1.38-1.18 (m, 6H). ESI-MS: 506.5 [M+H]⁺.

### Preparation of N¹-hydroxy-N⁶-(3-hydroxy-4-oxo-2-(2-(trifluoromethyl)phenyl)-1,4-dihydroquinolin-6-yl)adipamide (Cpd032)

Cpd032 was prepared following the general procedure described above using 39e (131 mg) as starting material. The desired product (Cpd032) was obtained as a beige solid (62 mg, 29% yield over 2 steps). ¹H NMR (500 MHz, DMSO-d6) δ 11.81 (s, 1H), 10.38 (s, 1H), 10.06 (s, 1H), 8.70 (br s, 1H), 8.45 (d, J=2.2 Hz, 1H), 8.27 (br s, 1H), 7.93 (d, J=7.9 Hz, 1H), 7.83 (t, J=7.5 Hz, 1H), 7.81-7.73 (m, 2H), 7.64 (d, J=7.5 Hz, 1H), 7.48 (d, J=9.0 Hz, 1H), 2.33 (t, J=7.0 Hz, 2H), 1.99 (t, J=7.0 Hz, 2H), 1.67-1.48 (m, 4H). ESI-MS: 464.2 [M+H]⁺.

### Preparation of N¹-hydroxy-N⁷-(3-hydroxy-4-oxo-2-(2-(trifluoromethyl)phenyl)-1,4-dihydroquinolin-6-yl)heptanediamide (Cpd033)

Cpd033 was prepared following the general procedure described above using 39f (123 mg) as starting material. The desired product (Cpd033) was obtained as a white solid (103 mg, 46% yield over 2 steps). ¹H NMR (500 MHz, DMSO-d6) δ 11.81 (s, 1H), 10.35 (s, 1H), 10.06 (s, 1H), 8.68 (s, 1H), 8.45 (d, J=2.2 Hz, 1H), 8.26 (br s, 1H), 7.93 (d, J=7.9 Hz, 1H), 7.84 (t, J=7.5 Hz, 1H), 7.80-7.73 (m, 2H), 7.64 (d, J=7.5 Hz, 1H), 7.48 (d, J=9.0 Hz, 1H), 2.32 (t, J=7.4 Hz, 2H), 1.96 (t, J=7.3 Hz, 2H), 1.67-1.57 (m, 2H), 1.57-1.48 (m, 2H), 1.35-1.24 (m, 2H). ESI-MS: 478.9 [M+H]⁺.

### Preparation of N¹-hydroxy-N⁸-(3-hydroxy-4-oxo-2-(2-(trifluoromethyl)phenyl)-1,4-dihydroquinolin-6-yl)octanediamide (Cpd034)

Cpd034 was prepared following the general procedure described above using 39g (134 mg) as starting material. The desired product (Cpd034) was obtained as a beige solid (15 mg, 11% yield over 2 steps). ¹H NMR (500 MHz, DMSO-d6) δ 11.81 (s, 1H), 10.35 (s, 1H), 10.05 (s, 1H), 8.68 (s, 1H), 8.45 (d, J=2.0 Hz, 1H), 8.26 (br s, 1H), 7.93 (d, J=7.9 Hz, 1H), 7.83 (t, J=7.5 Hz, 1H), 7.77 (d, J=8.4 Hz, 2H), 7.64 (d, J=7.5 Hz, 1H), 7.48 (d, J=9.0 Hz, 1H), 2.32 (t, J=7.3 Hz, 2H), 1.94 (t, J=7.3 Hz, 2H), 1.67-1.56 (m, 2H), 1.54-1.45 (m, 2H), 1.35-1.21 (m, 4H). ESI-MS: 492.2 [M+H]⁺.

### Preparation of N¹-hydroxy-N⁹-(3-hydroxy-4-oxo-2-(2-(trifluoromethyl)phenyl)-1,4-dihydroquinolin-6-yl)nonanediamide (Cpd035)

Cpd035 was prepared following the general procedure described above using 39h (195 mg) as starting material. The desired product (Cpd035) was obtained as a white solid (168 mg, 71% yield over 2 steps). ¹H NMR (500 MHz, DMSO-d6) δ 11.81 (br s, 1H), 10.34 (br s, 1H), 10.05 (s, 1H), 8.67 (br s, 1H), 8.45 (d, J=2.1 Hz, 1H), 8.27 (br s, 1H), 7.93 (d, J=7.9 Hz, 1H), 7.83 (t, J=7.5 Hz, 1H), 7.80-7.71 (m, 2H), 7.64 (d, J=7.5 Hz, 1H), 7.48 (d, J=9.0 Hz, 1H), 2.33 (t, J=7.4 Hz, 2H), 1.94 (t, J=7.3 Hz, 2H), 1.66-1.55 (m, 2H), 1.54-1.43 (m, 2H), 1.36-1.18 (m, 6H). ESI-MS: 506.1 [M+H]⁺.

### Preparation of N¹-hydroxy-N⁶-(3-hydroxy-4-oxo-2-(2-(trifluoromethyl)phenyl)-1,4-dihydroquinolin-7-yl)adipamide (Cpd036)

Cpd036 was prepared following the general procedure described above using 39i (147 mg) as starting material. The desired product (Cpd036) was obtained as a white solid (73 mg, 34% yield over 2 steps). ¹H NMR (500 MHz, DMSO-d6) δ 11.70 (s, 1H), 10.37 (s, 1H), 10.20 (s, 1H), 8.69 (s, 1H), 8.17 (br s, 1H), 8.12 (d, J=1.4 Hz, 1H), 8.06 (d, J=8.9 Hz, 1H), 7.91 (d, J=7.9 Hz, 1H), 7.82 (t, J=7.4 Hz, 1H), 7.75 (t, J=7.7 Hz, 1H), 7.62 (d, J=7.5 Hz, 1H), 7.26 (dd, J=8.9, 1.7 Hz, 1H), 2.35 (t, J=6.9 Hz, 2H), 1.97 (t, J=6.9 Hz, 2H), 1.65-1.47 (m, 4H). ESI-MS: 464.2 [M+H]⁺.

### Preparation of N¹-hydroxy-N⁷-(3-hydroxy-4-oxo-2-(2-(trifluoromethyl)phenyl)-1,4-dihydroquinolin-7-yl)heptanediamide (Cpd037)

Cpd037 was prepared following the general procedure described above using 39j (1.2 g) as starting material. The desired product (Cpd037) was obtained as a white solid (1.2 g, 78% yield over 2 steps). ¹H NMR (500 MHz, DMSO-d6) δ 11.69 (s, 1H), 10.34 (br s, 1H), 10.19 (s, 1H), 8.68 (br s, 1H), 8.31-8.09 (m, 2H), 8.06 (d, J=8.9 Hz, 1H), 7.91 (d, J=7.8 Hz, 1H), 7.82 (t, J=7.5 Hz, 1H), 7.75 (t, J=7.7 Hz, 1H), 7.61 (d, J=7.5 Hz, 1H), 7.26 (dd, J=8.9, 1.7 Hz, 1H), 2.34 (t, J=7.4 Hz, 2H), 1.94 (t, J=7.3 Hz, 2H), 1.59 (dt, J=15.1, 7.5 Hz, 2H), 1.51 (dt, J=15.1, 7.5 Hz, 2H), 1.27 (dt, J=15.2, 7.7 Hz, 2H). ESI-MS: 478.2 [M+H]⁺.

### Preparation of N¹-hydroxy-N⁸-(3-hydroxy-4-oxo-2-(2-(trifluoromethyl)phenyl)-1,4-dihydroquinolin-7-yl)octanediamide (Cpd038)

Cpd038 was prepared following the general procedure described above using 39k (50 mg) as starting material. The desired product (Cpd038) was obtained as a brown solid (16 mg, 32% yield over 2 steps). ¹H NMR (500 MHz, DMSO-d6) δ 12.06 (s, 1H), 10.09 (s, 3H), 8.20 (s, 1H), 8.09 (d, J=8.9 Hz, 1H), 7.92 (d, J=7.8 Hz, 1H), 7.83 (t, J=7.5 Hz, 1H), 7.76 (t, J=7.7 Hz, 1H), 7.63 (d, J=7.5 Hz, 1H), 7.34 (d, J=9.1 Hz, 1H), 2.36 (t, J=7.4 Hz, 2H), 1.93 (t, J=7.3 Hz, 2H), 1.63-1.54 (m, 2H), 1.54-1.42 (m, 2H), 1.36-1.18 (m, 4H). ESI-MS: 492.4 [M+H]⁺.

### Preparation of N¹-hydroxy-N⁹-(3-hydroxy-4-oxo-2-(2-(trifluoromethyl)phenyl)-1,4-dihydroquinolin-7-yl)nonanediamide (Cpd039)

Cpd039 was prepared following the general procedure described above using 39l (185 mg) as starting material. The desired product (Cpd039) was obtained as a beige solid (142 mg, 60% yield over 2 steps). ¹H NMR (500 MHz, DMSO-d6) δ 11.69 (s, 1H), 10.33 (br s, 1H), 10.19 (s, 1H), 8.66 (s, 1H), 8.27-8.10 (m, 2H), 8.06 (d, J=8.9 Hz, 1H), 7.91 (d, J=7.9 Hz, 1H), 7.82 (t, J=7.5 Hz, 1H), 7.75 (t, J=7.7 Hz, 1H), 7.62 (d, J=7.5 Hz, 1H), 7.26 (dd, J=8.9, 1.7 Hz, 1H), 2.34 (t, J=7.4 Hz, 2H), 1.92 (t, J=7.3 Hz, 2H), 1.66-1.52 (m, 2H), 1.52-1.42 (m, 2H), 1.36-1.17 (m, 6H). ESI-MS: 506.1 [M+H]⁺.

### Preparation of N¹-hydroxy-N⁶-(3-hydroxy-4-oxo-2-(2-(trifluoromethyl)phenyl)-1,4-dihydroquinolin-8-yl)adipamide (Cpd040)

Cpd040 was prepared following the general procedure described above using 39m (179 mg) as starting material. The desired product (Cpd040) was obtained as a brown solid (58 mg, 27% yield over 2 steps). ¹H NMR (500 MHz, DMSO-d6) δ 10.94 (s, 1H), 10.36 (s, 1H), 9.41 (s, 1H), 8.68 (br s, 1H), 8.44 (br s, 1H), 8.03 (d, J=7.0 Hz, 1H), 7.94 (d, J=7.4 Hz, 1H), 7.89-7.72 (m, 3H), 7.64 (d, J=7.5 Hz, 1H), 7.33-7.21 (m, 1H), 2.45-2.30 (m, 2H), 2.03-1.87 (m, 2H), 1.67-1.41 (m, 4H). ESI-MS: 464.2 [M+H]⁺.

### Preparation of N¹-hydroxy-N⁷-(3-hydroxy-4-oxo-2-(2-(trifluoromethyl)phenyl)-1,4-dihydroquinolin-8-yl)heptanediamide (Cpd041)

Cpd041 was prepared following the general procedure described above using 39n (170 mg) as starting material. The desired product (Cpd041) was obtained as a brown solid (61 mg, 27% yield over 2 steps). ¹H NMR (500 MHz, DMSO-d6) δ 10.92 (s, 1H), 10.33 (s, 1H), 9.41 (s, 1H), 8.67 (br s, 1H), 8.45 (br s, 1H), 8.03 (d, J=8.0 Hz, 1H), 7.94 (d, J=7.9 Hz, 1H), 7.87-7.81 (m, 2H), 7.81-7.75 (m, 1H), 7.64 (d, J=7.5 Hz, 1H), 7.27 (t, J=7.9 Hz, 1H), 2.37-2.30 (m, 2H), 1.93 (t, J=7.3 Hz, 2H), 1.64-1.40 (m, 4H), 1.34-1.19 (m, 2H). ESI-MS: 478.6 [M+H]⁺.

### Preparation of N¹-hydroxy-N⁸-(3-hydroxy-4-oxo-2-(2-(trifluoromethyl)phenyl)-1,4-dihydroquinolin-8-yl)octanediamide (Cpd042)

Cpd042 was prepared following the general procedure described above using 39o (83 mg) as starting material. The desired product (Cpd042) was obtained as a brown solid (32 mg, 10% yield over 2 steps). ¹H NMR (500 MHz, DMSO-d6) δ 10.90 (s, 1H), 10.33 (s, 1H), 9.43 (s, 1H), 8.67 (s, 1H), 8.44 (s, 1H), 8.11-7.98 (m, 1H), 7.94 (d, J=7.4 Hz, 1H), 7.90-7.71 (m, 2H), 7.64 (d, J=7.5 Hz, 1H), 7.28 (s, 1H), 2.35 (t, J=6.7 Hz, 2H), 1.91 (t, J=6.9 Hz, 2H), 1.65-1.51 (m, 2H), 1.51-1.39 (m, 2H), 1.34-1.16 (m, 4H). ESI-MS: 492.2 [M+H]⁺.

### Preparation of N¹-hydroxy-N⁹-(3-hydroxy-4-oxo-2-(2-(trifluoromethyl)phenyl)-1,4-dihydroquinolin-8-yl)nonanediamide (Cpd043)

Cpd043 was prepared following the general procedure described above using 39p (176 mg) as starting material. The desired product (Cpd043) was obtained as a beige solid (144 mg, 61% yield over 2 steps). ¹H NMR (500 MHz, DMSO-d6) δ 10.87 (br s, 1H), 10.33 (br s, 1H), 9.47 (br s, 1H), 8.97-8.19 (m, 2H), 8.12-7.89 (m, 2H), 7.89-7.79 (m, 2H), 7.79-7.68 (m, 1H), 7.64 (d, J=7.5 Hz, 1H), 7.40-7.20 (m, 1H), 2.36 (t, J=7.4 Hz, 2H), 1.91 (t, J=7.3 Hz, 2H), 1.63-1.51 (m, 2H), 1.51-1.39 (m, 2H), 1.35-1.13 (m, 6H). ESI-MS: 506.2 [M+H]⁺.

### Preparation of N-hydroxy-4-(2-((3-hydroxy-4-oxo-2-(2-(trifluoromethyl)phenyl)-1,4-dihydroquinolin-5-yl)amino)-2-oxoethyl)benzamide (Cpd044)

Cpd044 was prepared following the general procedure described above using 39q (119 mg) as starting material. The desired product (Cpd044) was obtained as a brown solid (30 mg, 19% yield over 2 steps). ¹H NMR (500 MHz, DMSO-d6) δ 13.94 (s, 1H), 12.28 (s, 1H), 11.20 (s, 1H), 9.03 (s, 1H), 8.64 (s, 1H), 8.28 (d, J=8.0 Hz, 1H), 7.96 (d, J=7.8 Hz, 1H), 7.86 (t, J=7.5 Hz, 1H), 7.80 (t, J=7.6 Hz, 1H), 7.73 (d, J=8.0 Hz, 2H), 7.67 (d, J=7.5 Hz, 1H), 7.49 (t, J=8.2 Hz, 1H), 7.45 (d, J=8.0 Hz, 2H), 7.20 (d, J=8.5 Hz, 1H), 3.81 (s, 2H). ESI-MS: 498.4 [M+H]⁺.

### Preparation of N-hydroxy-4-(2-((3-hydroxy-4-oxo-2-(2-(trifluoromethyl)phenyl)-1,4-dihydroquinolin-6-yl)amino)-2-oxoethyl)benzamide (Cpd045)

Cpd045 was prepared following the general procedure described above using 39r (144 mg) as starting material. The desired product (Cpd045) was obtained as a brown solid (83 mg, 53% yield over 2 steps). ¹H NMR (500 MHz, DMSO-d6) δ 11.82 (s, 1H), 11.19 (s, 1H), 10.40 (s, 1H), 9.02 (s, 1H), 8.44 (d, J=2.3 Hz, 1H), 8.29 (br s, 1H), 7.93 (d, J=7.8 Hz, 1H), 7.83 (t, J=7.5 Hz, 1H), 7.81-7.74 (m, 2H), 7.72 (d, J=8.2 Hz, 2H), 7.64 (d, J=7.6 Hz, 1H), 7.49 (d, J=9.1 Hz, 1H), 7.43 (d, J=8.2 Hz, 2H), 3.73 (s, 2H). ESI-MS: 498.2 [M+H]⁺.

### Preparation of N-hydroxy-4-(2-((3-hydroxy-4-oxo-2-(2-(trifluoromethyl)phenyl)-1,4-dihydroquinolin-7-yl)amino)-2-oxoethyl)benzamide (Cpd046)

Cpd046 was prepared following the general procedure described above using 39s (130 mg) as starting material. The desired product (Cpd046) was obtained as a brown solid (14 mg, 9% yield over 2 steps). ¹H NMR (500 MHz, DMSO-d6) δ 11.70 (s, 1H), 11.18 (s, 1H), 10.52 (s, 1H), 9.02 (s, 1H), 8.20 (br s, 1H), 8.12-8.05 (m, 2H), 7.91 (d, J=7.9 Hz, 1H), 7.82 (t, J=7.5 Hz, 1H), 7.74 (t, J=7.7 Hz, 1H), 7.71 (d, J=8.1 Hz, 2H), 7.61 (d, J=7.5 Hz, 1H), 7.41 (d, J=8.1 Hz, 2H), 7.29 (dd, J=8.9, 1.6 Hz, 1H), 3.75 (s, 2H). ESI-MS: 498.2 [M+H]⁺.

### Preparation of N-hydroxy-4-(2-((3-hydroxy-4-oxo-2-(2-(trifluoromethyl)phenyl)-1,4-dihydroquinolin-8-yl)amino)-2-oxoethyl)benzamide (Cpd047)

Cpd047 was prepared following the general procedure described above using 39t (160 mg) as starting material. The desired product (Cpd047) was obtained as a brown solid (57 mg, 37% yield over 2 steps). ¹H NMR (500 MHz, DMSO-d6) δ 11.41-10.74 (m, 2H), 9.77 (br s, 1H), 9.01 (br s, 1H), 8.47 (br s, 1H), 8.03 (br s, 1H), 7.98-7.90 (m, 1H), 7.90-7.71 (m, 2H), 7.71-7.54 (m, 2H), 7.38 (d, J=7.6 Hz, 2H), 7.33-7.20 (m, 1H), 3.77 (s, 2H). ESI-MS: 498.5 [M+H]⁺.

### Example 9

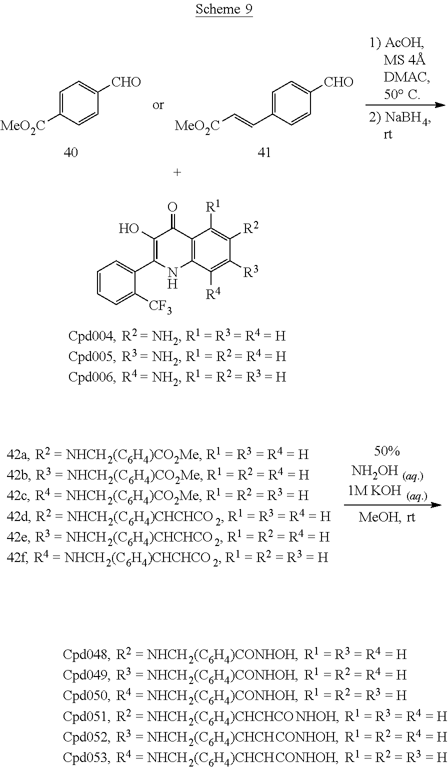

### General Procedure for the Preparation of Compounds 42a-f

To a mixture of anilines (1.0 equiv.), aldehyde 40 or 41 (1.2 equiv.) and 4 Å MS (molecular sieves) in DMAC (0.5 M) was added acetic acid (2.0 equiv.) at room temperature. The reaction mixture was stirred at 50° C. for 18 h under nitrogen atmosphere. The mixture was cooled on ice and crushed NaBH₄ (1.5 equiv.) was added with care. The resulting mixture was stirred at room temperature for 1 h. The mixture was poured into cold water and the precipitate was collected by filtration, rinsed with deionized water, and dried under vacuum. The crude product was purified by flash chromatography using a gradient elution of 1% to 10% MeOH in DCM. The desired product (42a-f) was obtained as a beige to brown solids.

### Preparation of methyl 4-(((3-hydroxy-4-oxo-2-(2-(trifluoromethyl)phenyl)-1,4-dihydroquinolin-6-yl)amino)methyl)benzoate (42a)

Compound 42a was prepared following the general procedure described above using Cpd004 (100 mg, 0.31 mmol) and aldehyde 40 (77 mg, 0.47 mmol) as starting materials. The desired product (42a) was obtained as a beige solid (52 mg, 36% yield). ESI-MS: 469.0 [M+H]⁺.

### Preparation of methyl 4-(((3-hydroxy-4-oxo-2-(2-(trifluoromethyl)phenyl)-1,4-dihydroquinolin-7-yl)amino)methyl)benzoate (42b)

Compound 42b was prepared following the general procedure described above using Cpd005 (1.0 g, 3.12 mmol) and aldehyde 40 (615 mg, 3.75 mmol) as starting materials. The desired product (42b) was obtained as a beige solid (1.4 g, 98% yield). ESI-MS: 469.2 [M+H]⁺.

### Preparation of methyl 4-(((3-hydroxy-4-oxo-2-(2-(trifluoromethyl)phenyl)-1,4-dihydroquinolin-8-yl)amino)methyl)benzoate (42c)

Compound 42c was prepared following the general procedure described above using Cpd006 (100 mg, 0.31 mmol) and aldehyde 40 (77 mg, 0.47 mmol) as starting materials. The desired product (42c) was obtained as a brown solid (91 mg, 62% yield). ESI-MS: 469.0 [M+H]⁺.

### Preparation of methyl (E)-3-(4-(((3-hydroxy-4-oxo-2-(2-(trifluoromethyl)phenyl)-1,4-dihydroquinolin-6-yl)amino)methyl)phenyl)acrylate (42d)

Compound 42d was prepared following the general procedure described above using Cpd004 (100 mg, 0.31 mmol) and aldehyde 41 (71 mg, 0.37 mmol) as starting materials. The desired product (42d) was obtained as a white solid (103 mg, 67% yield). ESI-MS: 495.0 [M+H]⁺.

### Preparation of methyl (E)-3-(4-(((3-hydroxy-4-oxo-2-(2-(trifluoromethyl)phenyl)-1,4-dihydroquinolin-7-yl)amino)methyl)phenyl)acrylate (42e)

Compound 42e was prepared following the general procedure described above using Cpd005 (100 mg, 0.31 mmol) and aldehyde 41 (71 mg, 0.37 mmol) as starting materials. The desired product (42e) was obtained as a pale yellow solid (83 mg, 54% yield). ESI-MS: 495.6 [M+H]⁺.

### Preparation of methyl (E)-3-(4-(((3-hydroxy-4-oxo-2-(2-(trifluoromethyl)phenyl)-1,4-dihydroquinolin-8-yl)amino)methyl)phenyl)acrylate (42f)

Compound 42f was prepared following the general procedure described above using Cpd006 (100 mg, 0.31 mmol) and aldehyde 41 (71 mg, 0.37 mmol) as starting materials. The desired product (42f) was obtained as a brown solid (27 mg, 17% yield). ESI-MS: 495.1 [M+H]⁺.

### Preparation of N-hydroxy-4-(((3-hydroxy-4-oxo-2-(2-(trifluoromethyl)phenyl)-1,4-dihydroquinolin-6-yl)amino)methyl)benzamide (Cpd048)

Cpd048 was prepared following the general procedure described above using 42a (52 mg, 0.11 mmol) as starting material. The desired product (Cpd048) was obtained as a brown solid (32 mg, 61% yield). ¹H NMR (500 MHz, DMSO-d6) δ 11.59 (s, 1H), 11.16 (s, 1H), 9.00 (br s, 1H), 7.91 (d, J=7.9 Hz, 1H), 7.81 (t, J=7.5 Hz, 1H), 7.75 (d, J=7.7 Hz, 1H), 7.71 (d, J=8.2 Hz, 2H), 7.58 (d, J=7.5 Hz, 1H), 7.46 (d, J=8.1 Hz, 2H), 7.34 (d, J=9.0 Hz, 1H), 7.11 (dd, J=9.0, 2.6 Hz, 1H), 7.01 (d, J=2.3 Hz, 1H), 6.53 (t, J=6.0 Hz, 1H), 4.48-4.32 (m, 2H). ESI-MS: 470.1 [M+H]⁺.

### Preparation of N-hydroxy-4-(((3-hydroxy-4-oxo-2-(2-(trifluoromethyl)phenyl)-1,4-dihydroquinolin-7-yl)amino)methyl)benzamide (Cpd049)

Cpd049 was prepared following the general procedure described above using 42b (900 mg, 1.92 mmol) as starting material. The desired product (Cpd049) was obtained as a brown solid (738 mg, 82% yield). ¹H NMR (500 MHz, DMSO-d6) δ 11.17 (s, 1H), 11.09 (s, 1H), 9.00 (br s, 1H), 7.88 (d, J=7.9 Hz, 1H), 7.83 (d, J=9.0 Hz, 1H), 7.78 (t, J=7.4 Hz, 1H), 7.74-7.68 (m, 3H), 7.54 (d, J=7.5 Hz, 1H), 7.41 (d, J=8.1 Hz, 2H), 7.09 (t, J=6.0 Hz, 1H), 6.71 (dd, J=9.0, 2.0 Hz, 1H), 6.26 (d, J=1.8 Hz, 1H), 4.41-4.28 (m, 2H). ESI-MS: 470.1 [M+H]⁺.

### Preparation of N-hydroxy-4-(((3-hydroxy-4-oxo-2-(2-(trifluoromethyl)phenyl)-1,4-dihydroquinolin-8-yl)amino)methyl)benzamide (Cpd050)

Cpd050 was prepared following the general procedure described above using 42c (91 mg, 0.19 mmol) as starting material. The desired product (Cpd050) was obtained as a brown solid (83 mg, 91% yield). ¹H NMR (500 MHz, DMSO-d6) δ 11.15 (br s, 1H), 10.80 (br s, 1H), 9.00 (br s, 1H), 8.27 (br s, 1H), 7.98-7.90 (m, 1H), 7.90-7.81 (m, 1H), 7.81-7.73 (m, 1H), 7.73-7.61 (m, 3H), 7.44 (d, J=7.0 Hz, 2H), 7.10-6.98 (m, 1H), 6.62 (d, J=6.3 Hz, 1H), 6.48 (s, 1H), 4.60-4.34 (m, 2H). ESI-MS: 470.1 [M+H]⁺.

### Preparation of (E)-N-hydroxy-3-(4-(((3-hydroxy-4-oxo-2-(2-(trifluoromethyl)phenyl)-1,4-dihydroquinolin-6-yl)amino)methyl)phenyl)acrylamide (Cpd051)

Cpd051 was prepared following the general procedure described above using 42d (103 mg, 0.21 mmol) as starting material. The desired product (Cpd051) was obtained as a yellow solid (39 mg, 38% yield). ¹H NMR (500 MHz, DMSO-d6) δ 11.58 (s, 1H), 10.75 (br s, 1H), 9.03 (br s, 1H), 7.91 (d, J=7.9 Hz, 1H), 7.81 (t, J=7.5 Hz, 1H), 7.74 (t, J=7.7 Hz, 1H), 7.58 (d, J=7.6 Hz, 1H), 7.53 (d, J=8.0 Hz, 2H), 7.47-7.39 (m, 3H), 7.33 (d, J=9.0 Hz, 1H), 7.11 (dd, J=9.0, 2.5 Hz, 1H), 7.02 (d, J=2.2 Hz, 1H), 6.50 (t, J=6.0 Hz, 1H), 6.42 (d, J=15.8 Hz, 1H), 4.38 (d, J=5.8 Hz, 2H). ESI-MS: 496.6 [M+H]⁺.

### Preparation of (E)-N-hydroxy-3-(4-(((3-hydroxy-4-oxo-2-(2-(trifluoromethyl)phenyl)-1,4-dihydroquinolin-7-yl)amino)methyl)phenyl)acrylamide (Cpd052)

Cpd052 was prepared following the general procedure described above using 42e (83 mg, 0.17 mmol) as starting material. The desired product (Cpd052) was obtained as a brown solid (77 mg, 93% yield). ¹H NMR (500 MHz, DMSO-d6) δ 11.09 (s, 1H), 10.74 (br s, 1H), 9.03 (br s, 1H), 7.88 (d, J=7.9 Hz, 1H), 7.83 (d, J=8.9 Hz, 1H), 7.78 (t, J=7.5 Hz, 1H), 7.71 (t, J=7.7 Hz, 1H), 7.57-7.49 (m, 3H), 7.46-7.35 (m, 3H), 7.07 (t, J=5.9 Hz, 1H), 6.73-6.68 (m, 1H), 6.41 (d, J=15.8 Hz, 1H), 6.26 (s, 1H), 4.39-4.26 (m, 2H). ESI-MS: 496.6 [M+H]⁺.

### Preparation of (E)-N-hydroxy-3-(4-(((3-hydroxy-4-oxo-2-(2-(trifluoromethyl)phenyl)-1,4-dihydroquinolin-8-yl)amino)methyl)phenyl)acrylamide (Cpd053)

Cpd053 was prepared following the general procedure described above using 42f (27 mg, 0.05 mmol) as starting material. The desired product (Cpd053) was obtained as a brown solid (22 mg, 81% yield). ¹H NMR (500 MHz, DMSO-d6) δ 10.94-10.56 (m, 2H), 9.03 (br s, 1H), 8.26 (br s, 1H), 7.98-7.89 (m, 1H), 7.89-7.80 (m, 1H), 7.80-7.71 (m, 1H), 7.69-7.60 (m, 1H), 7.52 (s, 2H), 7.47 (d, J=7.8 Hz, 1H), 7.44-7.34 (m, 3H), 7.09-7.00 (m, 1H), 6.69-6.60 (m, 1H), 6.50-6.36 (m, 2H), 4.53-4.36 (m, 2H). ESI-MS: 496.2 [M+H]⁺.

### Example 10

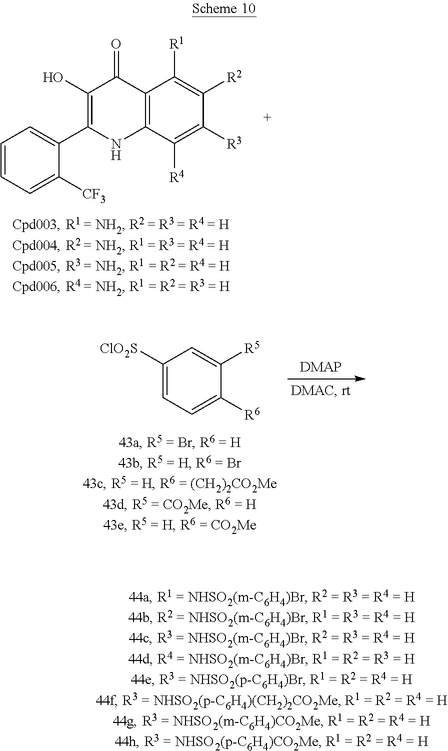

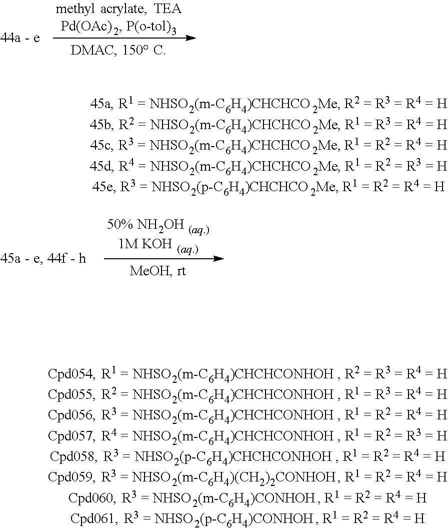

### General Procedure for the Preparation of Sulfonamide 44a-h

To a solution of anilines (1.0 equiv.) in DMAC (0.2 M) was added sulfonyl chloride 43a-e (1.1 equiv.) and DMAP (1.1 equiv.). The reaction mixture was stirred at room temperature for 16 h. Upon completion, the mixture was poured into cold water and the precipitate was collected by filtration, rinsed with deionized water, and dried under vacuum. The crude product was purified by flash chromatography using a gradient elution of 1% to 15% MeOH in DCM. The desired product (44a-h) was obtained as a beige to brown solids.

### Preparation of 3-bromo-N-(3-hydroxy-4-oxo-2-(2-(trifluoromethyl)phenyl)-1,4-dihydroquinolin-5-yl)benzenesulfonamide (44a)

Compound 44a was prepared following the general procedure described above using Cpd003 (100 mg, 0.31 mmol) and sulfonyl chloride 43a (88 mg, 0.34 mmol) as starting materials. The desired product (44a) was obtained as a brown solid (154 mg). ESI-MS: 539.4 [M+H]⁺.

### Preparation of 3-bromo-N-(3-hydroxy-4-oxo-2-(2-(trifluoromethyl)phenyl)-1,4-dihydroquinolin-6-yl)benzenesulfonamide (44b)

Compound 44b was prepared following the general procedure described above using Cpd004 (100 mg, 0.31 mmol) and sulfonyl chloride 43a (88 mg, 0.34 mmol) as starting materials. The desired product (44b) was obtained as a brown solid (171 mg). ESI-MS: 539.0 [M+H]⁺.

### Preparation of 3-bromo-N-(3-hydroxy-4-oxo-2-(2-(trifluoromethyl)phenyl)-1,4-dihydroquinolin-7-yl)benzenesulfonamide (44c)

Compound 44c was prepared following the general procedure described above using Cpd005 (1.0 g, 3.12 mmol) and sulfonyl chloride 43a (878 mg, 3.43 mmol) as starting materials. The desired product (44c) was obtained as a brown solid (790 mg). ESI-MS: 539.2 [M+H]⁺.

### Preparation of 3-bromo-N-(3-hydroxy-4-oxo-2-(2-(trifluoromethyl)phenyl)-1,4-dihydroquinolin-8-yl)benzenesulfonamide (44d)

Compound 44d was prepared following the general procedure described above using Cpd006 (300 mg, 0.94 mmol) and sulfonyl chloride 43a (263 mg, 1.03 mmol) as starting materials. The desired product (44d) was obtained as a brown solid (486 mg). ESI-MS: 539.6 [M+H]⁺.

### Preparation of 4-bromo-N-(3-hydroxy-4-oxo-2-(2-(trifluoromethyl)phenyl)-1,4-dihydroquinolin-7-yl)benzenesulfonamide (44e)

Compound 44e was prepared following the general procedure described above using Cpd005 (100 mg, 0.31 mmol) and sulfonyl chloride 43b (88 mg, 0.34 mmol) as starting materials. The desired product (44e) was obtained as a grey solid (148 mg). ESI-MS: 539.1 [M+H]⁺.

### Preparation of methyl 3-(4-(N-(3-hydroxy-4-oxo-2-(2-(trifluoromethyl)phenyl)-1,4-dihydroquinolin-7-yl)sulfamoyl)phenyl)propanoate (44f)

Compound 44f was prepared following the general procedure described above using Cpd005 (100 mg, 0.31 mmol) and sulfonyl chloride 43c (90 mg, 0.34 mmol) as starting materials. The desired product (44f) was obtained as a grey solid (164 mg). ESI-MS: 547.2 [M+H]⁺.

### Preparation of methyl 3-(N-(3-hydroxy-4-oxo-2-(2-(trifluoromethyl)phenyl)-1,4-dihydroquinolin-7-yl)sulfamoyl)benzoate (44g)

Compound 44g was prepared following the general procedure described above using Cpd005 (100 mg, 0.31 mmol) and sulfonyl chloride 43d (81 mg, 0.34 mmol) as starting materials. The desired product (44g) was obtained as a grey solid (154 mg). ESI-MS: 519.0 [M+H]⁺.

### Preparation of methyl 4-(N-(3-hydroxy-4-oxo-2-(2-(trifluoromethyl)phenyl)-1,4-dihydroquinolin-7-yl)sulfamoyl)benzoate (44h)

Compound 44h was prepared following the general procedure described above using Cpd005 (100 mg, 0.31 mmol) and sulfonyl chloride 43e (81 mg, 0.34 mmol) as starting materials. The desired product (44h) was obtained as a grey solid (156 mg). ESI-MS: 519.1 [M+H]⁺.

### General Procedure for the Preparation of Compounds 45a-e Via Heck Reaction

A mixture of aryl bromide 44a-e (1.0 equiv.), palladium acetate (5 mol %) and tris(o-tolyl)phosphine (10 mol %) in DMAC (0.3 M) was purged with nitrogen for 30 minutes. Methyl acrylate (2.0 equiv.) and triethylamine (2.5 equiv.) were then added. The reaction mixture was stirred at 150° C. for 7 h in a screw-cap tube. Upon completion, the mixture was cooled to room temperature and poured into cold water. The suspension was acidified to pH 5 with 2N HCl aqueous solution. The precipitate was collected by filtration, rinsed with deionized water, and dried under vacuum. The crude product was purified by flash chromatography using a gradient elution of 1% to 10% MeOH in DCM. The desired product (45a-e) was obtained as a beige to brown solids.

### Preparation of methyl (E)-3-(3-(N-(3-hydroxy-4-oxo-2-(2-(trifluoromethyl)phenyl)-1,4-dihydroquinolin-5-yl)sulfamoyl)phenyl)acrylate (45a)

Compound 45a was prepared following the general procedure described above using 44a (154 mg, 0.29 mmol) as starting material. The desired product (45a) was obtained as a brown solid (82 mg). ESI-MS: 545.5 [M+H]⁺.

### Preparation of methyl (E)-3-(3-(N-(3-hydroxy-4-oxo-2-(2-(trifluoromethyl)phenyl)-1,4-dihydroquinolin-6-yl)sulfamoyl)phenyl)acrylate (45b)

Compound 45b was prepared following the general procedure described above using 44b (171 mg, 0.32 mmol) as starting material. The desired product (45b) was obtained as a brown solid (123 mg). ESI-MS: 545.5 [M+H]⁺.

### Preparation of methyl (E)-3-(3-(N-(3-hydroxy-4-oxo-2-(2-(trifluoromethyl)phenyl)-1,4-dihydroquinolin-7-yl)sulfamoyl)phenyl)acrylate (45c)

Compound 45c was prepared following the general procedure described above using 44c (790 mg, 1.46 mmol) as starting material. The desired product (45c) was obtained as a white solid (313 mg). ESI-MS: 545.2 [M+H]⁺.

### Preparation of methyl (E)-3-(3-(N-(3-hydroxy-4-oxo-2-(2-(trifluoromethyl)phenyl)-1,4-dihydroquinolin-8-yl)sulfamoyl)phenyl)acrylate (45d)

Compound 45d was prepared following the general procedure described above using 44d (486 mg, 0.90 mmol) as starting material. The desired product (45d) was obtained as a brown solid (145 mg). ESI-MS: 545.7 [M+H]⁺.

### Preparation of methyl (E)-3-(4-(N-(3-hydroxy-4-oxo-2-(2-(trifluoromethyl)phenyl)-1,4-dihydroquinolin-7-yl)sulfamoyl)phenyl)acrylate (45e)

Compound 45e was prepared following the general procedure described above using 44e (148 mg, 0.27 mmol) as starting material. The desired product (45e) was obtained as a yellow solid (62 mg). ESI-MS: 545.1 [M+H]⁺.

### Preparation of (E)-N-hydroxy-3-(3-(N-(3-hydroxy-4-oxo-2-(2-(trifluoromethyl)phenyl)-1,4-dihydroquinolin-5-yl)sulfamoyl)phenyl)acrylamide (Cpd054)

Cpd054 was prepared following the general procedure described above using 45a (82 mg, 0.15 mmol) as starting material. The desired product (Cpd054) was obtained as a brown solid (36 mg, 21% yield over 3 steps). ¹H NMR (500 MHz, DMSO-d6) δ 12.43 (s, 1H), 10.79 (br s, 1H), 9.15 (br s, 1H), 8.76 (br s, 1H), 8.07 (s, 1H), 7.93 (d, J=7.8 Hz, 1H), 7.88-7.74 (m, 4H), 7.66-7.55 (m, 2H), 7.53-7.41 (m, 2H), 7.22-7.11 (m, 2H), 6.54 (d, J=15.9 Hz, 1H). ESI-MS: 546.6 [M+H]⁺.

### Preparation of (E)-N-hydroxy-3-(3-(N-(3-hydroxy-4-oxo-2-(2-(trifluoromethyl)phenyl)-1,4-dihydroquinolin-6-yl)sulfamoyl)phenyl)acrylamide (Cpd055)

Cpd055 was prepared following the general procedure described above using 45b (123 mg, 0.23 mmol) as starting material. The desired product (Cpd055) was obtained as a brown solid (48 mg, 28% yield over 3 steps). ¹H NMR (500 MHz, DMSO-d6) δ 11.86 (br s, 1H), 10.84 (br s, 1H), 10.43 (br s, 1H), 9.15 (br s, 1H), 8.31 (br s, 1H), 7.98 (s, 1H), 7.91 (d, J=7.8 Hz, 1H), 7.85-7.72 (m, 4H), 7.70 (d, J=7.9 Hz, 1H), 7.63-7.54 (m, 2H), 7.52-7.43 (m, 2H), 7.40 (dd, J=9.0, 1.9 Hz, 1H), 6.54 (d, J=15.9 Hz, 1H). ESI-MS: 546.4 [M+H]⁺.

### Preparation of (E)-N-hydroxy-3-(3-(N-(3-hydroxy-4-oxo-2-(2-(trifluoromethyl)phenyl)-1,4-dihydroquinolin-7-yl)sulfamoyl)phenyl)acrylamide (Cpd056)

Cpd056 was prepared following the general procedure described above using 45c (313 mg, 0.57 mmol) as starting material. The desired product (Cpd056) was obtained as a beige solid (303 mg, 18% yield over 3 steps). ¹H NMR (500 MHz, DMSO-d6) δ 11.65 (s, 1H), 11.05-10.69 (m, 2H), 9.15 (br s, 1H), 8.22 (br s, 1H), 8.04-7.97 (m, 2H), 7.91 (d, J=7.8 Hz, 1H), 7.85-7.77 (m, 3H), 7.75 (t, J=7.6 Hz, 1H), 7.63-7.56 (m, 2H), 7.48 (d, J=15.7 Hz, 1H), 7.30 (s, 1H), 7.02 (dd, J=8.9, 1.5 Hz, 1H), 6.52 (d, J=15.9 Hz, 1H). ESI-MS: 546.6 [M+H]⁺.

### Preparation of (E)-N-hydroxy-3-(3-(N-(3-hydroxy-4-oxo-2-(2-(trifluoromethyl)phenyl)-1,4-dihydroquinolin-8-yl)sulfamoyl)phenyl)acrylamide (Cpd057)

Cpd057 was prepared following the general procedure described above using 45d (145 mg, 0.27 mmol) as starting material. The desired product (Cpd057) was obtained as a grey solid (96 mg, 19% yield over 3 steps). ¹H NMR (500 MHz, DMSO-d6) δ 10.86-10.75 (m, 2H), 9.81 (br s, 1H), 9.17 (s, 1H), 8.50 (br s, 1H), 8.11-7.97 (m, 1H), 7.91 (d, J=7.7 Hz, 1H), 7.88-7.72 (m, 4H), 7.53-7.42 (m, 3H), 7.36 (d, J=6.7 Hz, 1H), 7.30-7.24 (m, 1H), 7.24-7.17 (m, 1H), 6.48 (d, J=15.8 Hz, 1H). ESI-MS: 546.1 [M+H]⁺.

### Preparation of (E)-N-hydroxy-3-(4-(N-(3-hydroxy-4-oxo-2-(2-(trifluoromethyl)phenyl)-1,4-dihydroquinolin-7-yl)sulfamoyl)phenyl)acrylamide (Cpd058)

Cpd058 was prepared following the general procedure described above using 45e (62 mg, 0.11 mmol) as starting material. The desired product (Cpd058) was obtained as a beige solid (35 mg, 21% yield over 3 steps). ¹H NMR (500 MHz, DMSO-d6) δ 11.67 (s, 1H), 10.99-10.76 (m, 2H), 9.15 (s, 1H), 8.24 (br s, 1H), 8.00 (d, J=8.9 Hz, 1H), 7.91 (d, J=7.8 Hz, 1H), 7.85 (d, J=8.2 Hz, 2H), 7.81 (t, J=7.5 Hz, 1H), 7.78-7.71 (m, 3H), 7.60 (d, J=7.5 Hz, 1H), 7.47 (d, J=15.8 Hz, 1H), 7.31 (s, 1H), 7.00 (dd, J=8.9, 1.7 Hz, 1H), 6.53 (d, J=15.8 Hz, 1H). ESI-MS: 546.2 [M+H]⁺.

### Preparation of N-hydroxy-3-(4-(N-(3-hydroxy-4-oxo-2-(2-(trifluoromethyl)phenyl)-1,4-dihydroquinolin-7-yl)sulfamoyl)phenyl)propanamide (Cpd059)

Cpd059 was prepared following the general procedure described above using 44f (164 mg, 0.30 mmol) as starting material. The desired product (Cpd059) was obtained as a beige solid (76 mg, 45% yield over 2 steps). ¹H NMR (500 MHz, DMSO-d6) δ 11.67 (s, 1H), 10.81 (s, 1H), 10.36 (s, 1H), 8.72 (s, 1H), 8.23 (br s, 1H), 7.99 (d, J=8.9 Hz, 1H), 7.91 (d, J=7.8 Hz, 1H), 7.82 (t, J=7.5 Hz, 1H), 7.79-7.72 (m, 3H), 7.60 (d, J=7.5 Hz, 1H), 7.39 (d, J=8.2 Hz, 2H), 7.33 (d, J=1.7 Hz, 1H), 7.00 (dd, J=8.9, 1.9 Hz, 1H), 2.84 (t, J=7.5 Hz, 2H), 2.25 (t, J=7.6 Hz, 2H). ESI-MS: 548.1 [M+H]⁺.

### Preparation of N-hydroxy-3-(N-(3-hydroxy-4-oxo-2-(2-(trifluoromethyl)phenyl)-1,4-dihydroquinolin-7-yl)sulfamoyl)benzamide (Cpd060)

Cpd060 was prepared following the general procedure described above using 44g (154 mg, 0.30 mmol) as starting material. The desired product (Cpd060) was obtained as a beige solid (61 mg, 38% yield over 2 steps). ¹H NMR (500 MHz, DMSO-d6) δ 11.68 (s, 1H), 11.45 (br s, 1H), 10.86 (br s, 1H), 9.22 (s, 1H), 8.31-8.17 (m, 2H), 8.00 (d, J=8.9 Hz, 1H), 7.98-7.93 (m, 2H), 7.91 (d, J=7.8 Hz, 1H), 7.81 (t, J=7.5 Hz, 1H), 7.75 (t, J=7.7 Hz, 1H), 7.65 (t, J=7.8 Hz, 1H), 7.59 (d, J=7.5 Hz, 1H), 7.30 (d, J=1.8 Hz, 1H), 7.02 (dd, J=8.9, 2.0 Hz, 1H). ESI-MS: 520.1 [M+H]⁺.

### Preparation of N-hydroxy-4-(N-(3-hydroxy-4-oxo-2-(2-(trifluoromethyl)phenyl)-1,4-dihydroquinolin-7-yl)sulfamoyl)benzamide (Cpd061)

Cpd061 was prepared following the general procedure described above using 44h (156 mg, 0.30 mmol) as starting material. The desired product (Cpd061) was obtained as a beige solid (58 mg, 36% yield over 2 steps). ¹H NMR (500 MHz, DMSO-d6) δ 11.67 (s, 1H), 11.38 (s, 1H), 10.96 (s, 1H), 9.23 (s, 1H), 8.24 (br s, 1H), 8.01 (d, J=8.9 Hz, 1H), 7.94-7.90 (m, 3H), 7.88 (d, J=8.5 Hz, 2H), 7.81 (t, J=7.6 Hz, 1H), 7.75 (t, J=7.7 Hz, 1H), 7.60 (d, J=7.5 Hz, 1H), 7.33 (d, J=1.7 Hz, 1H), 7.01 (dd, J=8.9, 1.9 Hz, 1H). ESI-MS: 520.5 [M+H]⁺.

### Example 11

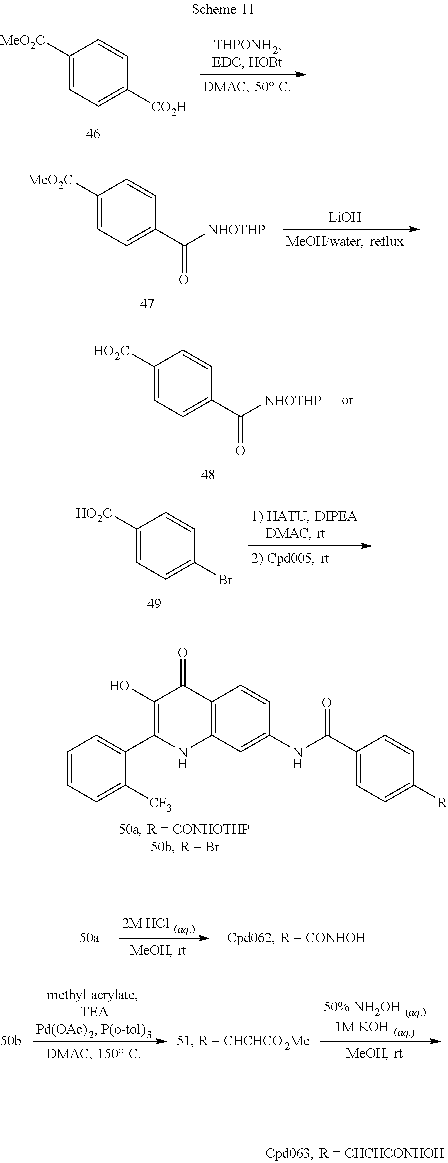

### Preparation of methyl 4-(((tetrahydro-2H-pyran-2-yl)oxy)carbamoyl)benzoate (47)

A mixture of 46 (1.0 g, 5.55 mmol), EDC (1.3 g, 6.66 mmol) and HOBt (1.4 g, 8.33 mmol) in 10 mL of DMAC was stirred at room temperature for 30 mins. NH₂OTHP (975 mg, 8.33 mmol) was added to the mixture and stirred at 50° C. for 24 h. Upon completion, the reaction mixture was poured into 100 mL of cold water. The resulting white precipitate was collected by filtration, rinsed with deionized water, and dried under vacuum. The desired product (47) was obtained as a white solid (1.7 g, >99% yield).

### Preparation of 4-(((tetrahydro-2H-pyran-2-yl)oxy)carbamoyl)benzoic acid (48)

A mixture of 47 (800 mg, 2.86 mmol) and LiOH (343 mg, 14.32 mmol) in 8 mL of 3:1 water/MeOH was refluxed for 1 h. Upon completion, the reaction solution was cooled to rt and washed with DCM. The aqueous layer was acidified to pH 4 with 2N HCl aqueous solution with care. The resulting white precipitate was collected by filtration, rinsed with small amounts of deionized water, and dried under vacuum. The desired product (48) was obtained as a white solid (267 mg, 35% yield).

### General Procedure for the Preparation of Amides 50a, b

A mixture of benzoic acids 48 or 49 (1.2 equiv.), DIPEA (1.2 equiv.) and HATU (1.2 equiv.) in DMAC (0.5 M) was stirred at room temperature for 30 minutes. Cpd005 (1.0 equiv.) was then added to the solution and stirred at rt for 16 h. Upon completion, the reaction mixture was poured into 10 mL of cold water. The resulting precipitate was collected by filtration, rinsed with deionized water, and dried under vacuum. The crude product was purified by flash chromatography using a gradient elution of 0% to 10% MeOH in DCM. The desired product (50a, b) was obtained as grey solids.

### Preparation of N¹-(3-hydroxy-4-oxo-2-(2-(trifluoromethyl)phenyl)-1,4-dihydroquinolin-7-yl)-N⁴-((tetrahydro-2H-pyran-2-yl)oxy)terephthalamide (50a)

Compound 50a was prepared following the general procedure described above using Cpd005 (100 mg, 0.31 mmol) and 48 (99 mg, 0.37 mmol) as starting materials. The desired product (50a) was obtained as a grey solid (181 mg). ESI-MS: 568.3 [M+H]⁺.

### Preparation of 4-bromo-N-(3-hydroxy-4-oxo-2-(2-(trifluoromethyl)phenyl)-1,4-dihydroquinolin-7-yl)benzamide (50b)

Compound 50b was prepared following the general procedure described above using Cpd005 (150 mg, 0.47 mmol) and 49 (113 mg, 0.56 mmol) as starting materials. The desired product (50b) was obtained as a grey solid (272 mg). ESI-MS: 503.1 [M+H]⁺.

### Preparation of N¹-hydroxy-N⁴-(3-hydroxy-4-oxo-2-(2-(trifluoromethyl)phenyl)-1,4-dihydroquinolin-7-yl)terephthalamide (Cpd062)

To a solution of 50a (181 mg, 0.32 mmol) in 1 mL of MeOH was added dropwise 2N HCl aqueous solution on an ice bath. The reaction mixture was stirred at room temperature for 1 h. Upon completion, the solvent was removed by reduced pressure and the residue was suspended in water. The suspension was adjusted to pH 6 with 1N KOH aqueous solution on ice. The precipitate was collected by filtration, rinsed with deionized water, and dried under vacuum. The crude product was purified by flash chromatography using a gradient elution of 10% to 30% MeOH in DCM. The desired product (Cpd062) was obtained as a beige solid (50 mg, 33% yield over 2 steps). ESI-MS: 484.2 [M+H]⁺.

### Preparation of methyl (E)-3-(4-((3-hydroxy-4-oxo-2-(2-(trifluoromethyl)phenyl)-1,4-dihydroquinolin-7-yl)carbamoyl)phenyl)acrylate (51)

Compound 51 was prepared following the general procedure of Heck reaction described above using 50b (272 mg, 0.54 mmol) as starting material. The desired product (51) was obtained as a beige solid (14 mg, 6% yield over 2 steps). ESI-MS: 509.6 [M+H]⁺.

### Preparation of (E)-N-(3-hydroxy-4-oxo-2-(2-(trifluoromethyl)phenyl)-1,4-dihydroquinolin-7-yl)-4-(3-(hydroxyamino)-3-oxoprop-1-en-1-yl)benzamide (Cpd063)

Compound Cpd063 was prepared following the general procedure described above using 51 (14 mg, 0.03 mmol) as starting material. The desired product (Cpd063) was obtained as a brown solid (11 mg, 82% yield). ¹H NMR (500 MHz, DMSO-d6) δ 11.80 (s, 1H), 10.86 (br s, 1H), 10.59 (s, 1H), 9.15 (br s, 1H), 8.30 (s, 1H), 8.24 (br s, 1H), 8.13 (d, J=8.9 Hz, 1H), 8.01 (d, J=8.1 Hz, 2H), 7.93 (d, J=7.9 Hz, 1H), 7.84 (t, J=7.5 Hz, 1H), 7.79-7.70 (m, 3H), 7.65 (d, J=7.5 Hz, 1H), 7.58-7.50 (m, 2H), 6.59 (d, J=15.9 Hz, 1H). ESI-MS: 510.2 [M+H]⁺.

### Example 12

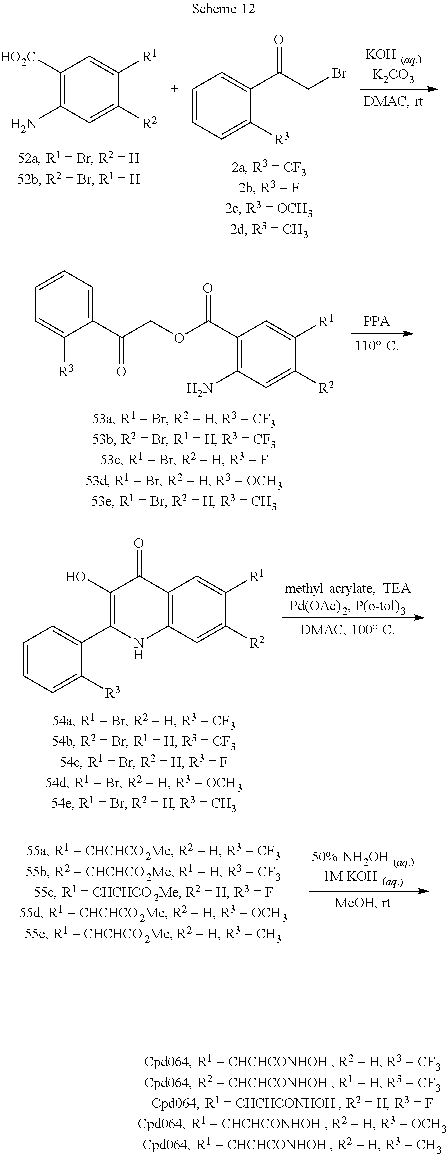

### Preparation of 2-oxo-2-(2-(trifluoromethyl)phenyl)ethyl 2-amino-5-bromobenzoate (53a)

To a solution of 2-amino-5-bromobenzoic acid (52a, 2.8 g, 13 mmol) in 5 mL of DMAC, an aqueous solution of KOH (820 mg, 17 mmol, dissolved in 1.3 mL of water) and K₂CO₃ (512 mg, 4.3 mmol) was added while stirring vigorously on an ice bath. The telescoped 2-bromo-1-(2-(trifluoromethyl)phenyl)ethan-1-one (2a, 3.0 g) was then added dropwise to the reaction mixture, which was maintained at ice bath temperature and stirred at room temperature for one hour. Upon completion, the reaction mixture was poured into 50 mL of ice-cold water and the resulting precipitate was collected by filtration. The wet crude product was suspended in 50 mL of 4:6 MeOH/water solution and stirred at room temperature for one hour. The crude product was collected by filtration and dried under reduced pressure. After repeating the process, the desired product (53a) was obtained as a brown solid (1.6 g) and was used directly in the next step without further purification.

### Preparation of 2-oxo-2-(2-(trifluoromethyl)phenyl)ethyl 2-amino-4-bromobenzoate (53b)

The procedure for 53b was identical to that described for 53a, except that 2-amino-4-bromobenzoic acid (52b, 2.8 g, 13 mmol) was used as the starting material. The desired product (53b) was obtained as a brown solid (3.9 g).

### Preparation of 2-(2-fluorophenyl)-2-oxoethyl 2-amino-5-bromobenzoate (53c)

The procedure for 53c was identical to that described for 53a, except that 2-bromo-1-(2-fluorophenyl)ethan-1-one (2b, 3.0 g, 14 mmol) was used as the starting material. The desired product (53c) was obtained as a yellow solid (3.8 g, 78% yield). ESI-MS: 352.5 [M+H]⁺.

### Preparation of 2-(2-methoxyphenyl)-2-oxoethyl 2-amino-5-bromobenzoate (53d)

The procedure for 53d was identical to that described for 53a, except that 2-bromo-1-(2-methoxyphenyl)ethan-1-one (2c, 3.0 g, 13 mmol) was used as the starting material. The desired product (53d) was obtained as a beige solid (3.8 g, 79% yield). ESI-MS: 364.5 [M+H]⁺.

### Preparation of 2-oxo-2-(o-tolyl)ethyl 2-amino-5-bromobenzoate (53e)

The procedure for 53e was identical to that described for 53a, except that 2-bromo-1-(o-tolyl)ethan-1-one (2d, 3.0 g, 14 mmol) was used as the starting material. The desired product (53e) was obtained as a beige solid (4.3 g, 87% yield). ESI-MS: 348.5 [M+H]⁺.

### Preparation of 6-bromo-3-hydroxy-2-(2-(trifluoromethyl)phenyl)quinolin-4(1H)-one (54a)

A mixture of 2-oxo-2-(2-(trifluoromethyl)phenyl)ethyl 2-amino-5-bromobenzoate (53a, 1.6 g) and 5 mL of PPA was stirred at 110° C. for 3 hours. Upon completion, the reaction mixture was poured into crushed ice and stirred for one hour. The resulting precipitate was collected by filtration, rinsed with deionized water, and dried under reduced pressure. The crude product was then suspended in 40 mL of 1:1 EtOAc/hexanes solution and stirred at room temperature for one hour. The solid was collected by filtration and dried under reduced pressure to obtain the desired product (54a) as a white solid (2.2 g, 53% yield over 3 steps). ESI-MS: 384.5 [M+H]⁺.

### Preparation of 7-bromo-3-hydroxy-2-(2-(trifluoromethyl)phenyl)quinolin-4(1H)-one (54b)

The procedure for 54b was identical to that described for 54a, except that 2-oxo-2-(2-(trifluoromethyl)phenyl)ethyl 2-amino-4-bromobenzoate (53b, 3.9 g) was used as the starting material. The desired product (54b) was obtained as a grey solid (2.8 g, 66% yield over 3 steps). ESI-MS: 384.4 [M+H]⁺.

### Preparation of 6-bromo-2-(2-fluorophenyl)-3-hydroxyquinolin-4(1H)-one (54c)

The procedure for 54c was identical to that described for 54a, except that 2-(2-fluorophenyl)-2-oxoethyl 2-amino-5-bromobenzoate (53c, 3.8 g) was used as the starting material. The desired product (54c) was obtained as a pale brown solid (2.9 g, 82% yield). ESI-MS: 334.3 [M+H]⁺.

### Preparation of 6-bromo-3-hydroxy-2-(2-methoxyphenyl)quinolin-4(1H)-one (54d)

The procedure for 54d was identical to that described for 54a, except that 2-(2-methoxyphenyl)-2-oxoethyl 2-amino-5-bromobenzoate (53d, 3.8 g) was used as the starting material. The desired product (54d) was obtained as a brown solid (3.6 g, 99% yield). ESI-MS: 346.3 [M+H]⁺.

### Preparation of 6-bromo-3-hydroxy-2-(o-tolyl)quinolin-4(1H)-one (54e)

The procedure for 54e was identical to that described for 54a, except that 2-oxo-2-(o-tolyl)ethyl 2-amino-5-bromobenzoate (53e, 4.2 g) was used as the starting material. The desired product (54e) was obtained as a beige solid (3.9 g, 98% yield). ESI-MS: 330.3 [M+H]⁺.

### Preparation of methyl (E)-3-(3-hydroxy-4-oxo-2-(2-(trifluoromethyl)phenyl)-1,4-dihydroquinolin-6-yl)acrylate (55a)

A mixture of 6-bromo-3-hydroxy-2-(2-(trifluoromethyl)phenyl)quinolin-4(1H)-one (54a, 200 mg, 0.52 mmol), palladium acetate (6 mg, 5 mol %) and tris(o-tolyl)phosphine (16 mg, 10 mol %) in 2 mL of DMAC was purged with nitrogen for 30 minutes. Methyl acrylate (94 μL, 1.04 mmol) and triethylamine (181 μL, 1.30 mmol) were then added. The reaction mixture was stirred at 100° C. for 3 h in a screw-cap tube. Upon completion, the mixture was cooled to room temperature, filtered through a pad of silica gel, and eluted with MeOH. The collected suspension was concentrated by rotavapor, and the residue was washed sequentially with deionized water, a 1:1 EtOAc/hexanes solution, and a 1:1 MeOH/water solution, then dried under vacuum. The desired product (55a) was obtained as a brown solid (101 mg, 50% yield). ESI-MS: 390.0 [M+H]⁺.

### Preparation of methyl (E)-3-(3-hydroxy-4-oxo-2-(2-(trifluoromethyl)phenyl)-1,4-dihydroquinolin-7-yl)acrylate (55b)

The procedure for 55b was identical to that described for 55a, except that 7-bromo-3-hydroxy-2-(2-(trifluoromethyl)phenyl)quinolin-4(1H)-one (54b, 1.4 g) was used as the starting material. The desired product (55b) was obtained as a brown solid (823 mg, 58% yield). ESI-MS: 390.0 [M+H]⁺.

### Preparation of methyl (E)-3-(2-(2-fluorophenyl)-3-hydroxy-4-oxo-1,4-dihydroquinolin-6-yl)acrylate (55c)

The procedure for 55c was identical to that described for 55a, except that 6-bromo-2-(2-fluorophenyl)-3-hydroxyquinolin-4(1H)-one (54c, 2.9 g) was used as the starting material. After the reaction was complete, the reaction mixture was allowed to cool to room temperature and subsequently poured into 150 mL of water. The resulting precipitate was filtered and re-suspended in a solution containing 3 g of citric acid and 30 mL of a 4:1 THF/MeOH mixture. The suspension was stirred at room temperature for 2 hours. The crude product was then collected by filtration and washed with a small amount of the 4:1 THF/MeOH mixture. The desired product (55c) was obtained as a green solid (2.6 g, 85% yield). ESI-MS: 340.1 [M+H]⁺.

### Preparation of methyl (E)-3-(3-hydroxy-2-(2-methoxyphenyl)-4-oxo-1,4-dihydroquinolin-6-yl)acrylate (55d)

The procedure for 55d was identical to that described for 55a, except that 6-bromo-3-hydroxy-2-(2-methoxyphenyl)quinolin-4(1H)-one (54d, 2.0 g) was used as the starting material. After the reaction was complete, the reaction mixture was filtered through a silica gel pad while hot. The filtrate was then poured into 100 mL of water, the resulting precipitate was collected by filtration. The desired product (55d) was obtained as a brown solid (738 mg, 36% yield). ESI-MS: 352.0 [M+H]⁺.

### Preparation of methyl (E)-3-(3-hydroxy-4-oxo-2-(o-tolyl)-1,4-dihydroquinolin-6-yl)acrylate (55e)

The procedure for 55e was identical to that described for 55a, except that 6-bromo-3-hydroxy-2-(o-tolyl)quinolin-4(1H)-one (54e, 2.0 g) was used as the starting material. After the reaction was completed, the reaction mixture was allowed to cool to room temperature and subsequently poured into 100 mL of water. The resulting precipitate was filtered and re-suspended in a solution containing 2 g of citric acid and 20 mL of a 4:1 THF/MeOH mixture. The suspension was stirred at room temperature for 2 hours. The crude product was then collected by filtration and washed with a small amount of the 4:1 THF/MeOH mixture. The desired product (55e) was obtained as a green solid (1.6 g, 78% yield). ESI-MS: 336.0 [M+H]⁺.

### Preparation of (E)-N-hydroxy-3-(3-hydroxy-4-oxo-2-(2-(trifluoromethyl)phenyl)-1,4-dihydroquinolin-6-yl)acrylamide (Cpd064)

Cpd064 was prepared following the general procedure described above using 55a (101 mg, 0.26 mmol) as starting material. The desired product (Cpd064) was obtained as a brown solid (90 mg, 89% yield). ¹H NMR (500 MHz, DMSO-d6) δ 11.99 (br s, 1H), 10.75 (br s, 1H), 9.07 (br s, 1H), 8.50 (br s, 1H), 8.30 (s, 1H), 7.94 (d, J=7.9 Hz, 1H), 7.85 (t, J=7.5 Hz, 1H), 7.81-7.75 (m, 2H), 7.66 (d, J=7.5 Hz, 1H), 7.60-7.50 (m, 2H), 6.53 (d, J=15.8 Hz, 1H). ESI-MS: 391.6 [M+H]⁺.

### Preparation of (E)-N-hydroxy-3-(3-hydroxy-4-oxo-2-(2-(trifluoromethyl)phenyl)-1,4-dihydroquinolin-7-yl)acrylamide (Cpd065)

Cpd065 was prepared following the general procedure described above using 55b (100 mg, 0.26 mmol) as starting material. The desired product (Cpd065) was obtained as a brown solid (84 mg, 84% yield). ¹H NMR (500 MHz, DMSO-d6) δ 11.96 (br s, 1H), 10.88 (br s, 1H), 9.14 (br s, 1H), 8.46 (br s, 1H), 8.18 (d, J=8.6 Hz, 1H), 7.95 (d, J=7.9 Hz, 1H), 7.85 (t, J=7.5 Hz, 1H), 7.78 (t, J=7.7 Hz, 1H), 7.68-7.59 (m, 2H), 7.52 (d, J=15.8 Hz, 1H), 7.47 (d, J=8.6 Hz, 1H), 6.57 (d, J=15.8 Hz, 1H). ESI-MS: 391.5 [M+H]⁺.

### Preparation of (E)-3-(2-(2-fluorophenyl)-3-hydroxy-4-oxo-1,4-dihydroquinolin-6-yl)-N-hydroxyacrylamide (Cpd066)

Cpd066 was prepared following the general procedure described above using 55c (100 mg, 0.29 mmol) as starting material. The desired product (Cpd066) was obtained as a beige solid (80 mg, 80% yield). ¹H NMR (500 MHz, DMSO-d6) δ 11.78 (br s, 1H), 10.71 (br s, 1H), 9.03 (br s, 2H), 8.29 (s, 1H), 7.78 (d, J=8.1 Hz, 1H), 7.69-7.51 (m, 4H), 7.45-7.35 (m, 2H), 6.53 (d, J=15.8 Hz, 1H). ESI-MS: 341.4 [M+H]⁺.

### Preparation of (E)-N-hydroxy-3-(3-hydroxy-2-(2-methoxyphenyl)-4-oxo-1,4-dihydroquinolin-6-yl)acrylamide (Cpd067)

Cpd067 was prepared following the general procedure described above using 55d (738 mg, 2.10 mmol) as starting material. The desired product (Cpd067) was obtained as a brown solid (527 mg, 71% yield). ¹H NMR (500 MHz, DMSO-d6) δ 11.75 (s, 1H), 10.72 (s, 1H), 9.03 (s, 1H), 8.28 (s, 1H), 8.20 (br s, 1H), 7.75 (d, J=8.4 Hz, 1H), 7.61-7.48 (m, 3H), 7.43 (dd, J=7.5, 1.6 Hz, 1H), 7.21 (d, J=8.4 Hz, 1H), 7.10 (t, J=7.5 Hz, 1H), 6.52 (d, J=15.8 Hz, 1H), 3.78 (s, 3H). ESI-MS: 353.4 [M+H]⁺.

### Preparation of (E)-N-hydroxy-3-(3-hydroxy-4-oxo-2-(o-tolyl)-1,4-dihydroquinolin-6-yl)acrylamide (Cpd068)

Cpd068 was prepared following the general procedure described above using 55e (200 mg, 0.60 mmol) as starting material. The desired product (Cpd068) was obtained as a white solid (181 mg, 90% yield). ¹H NMR (500 MHz, DMSO-d6) δ 11.75 (br s, 1H), 10.74 (br s, 1H), 9.05 (br s, 1H), 8.61-8.10 (m, 2H), 7.77 (d, J=8.7 Hz, 1H), 7.60 (d, J=8.8 Hz, 1H), 7.55 (d, J=15.8 Hz, 1H), 7.47-7.31 (m, 4H), 6.53 (d, J=15.8 Hz, 1H), 2.22 (s, 3H). ESI-MS: 337.3 [M+H]⁺.

### Example 13

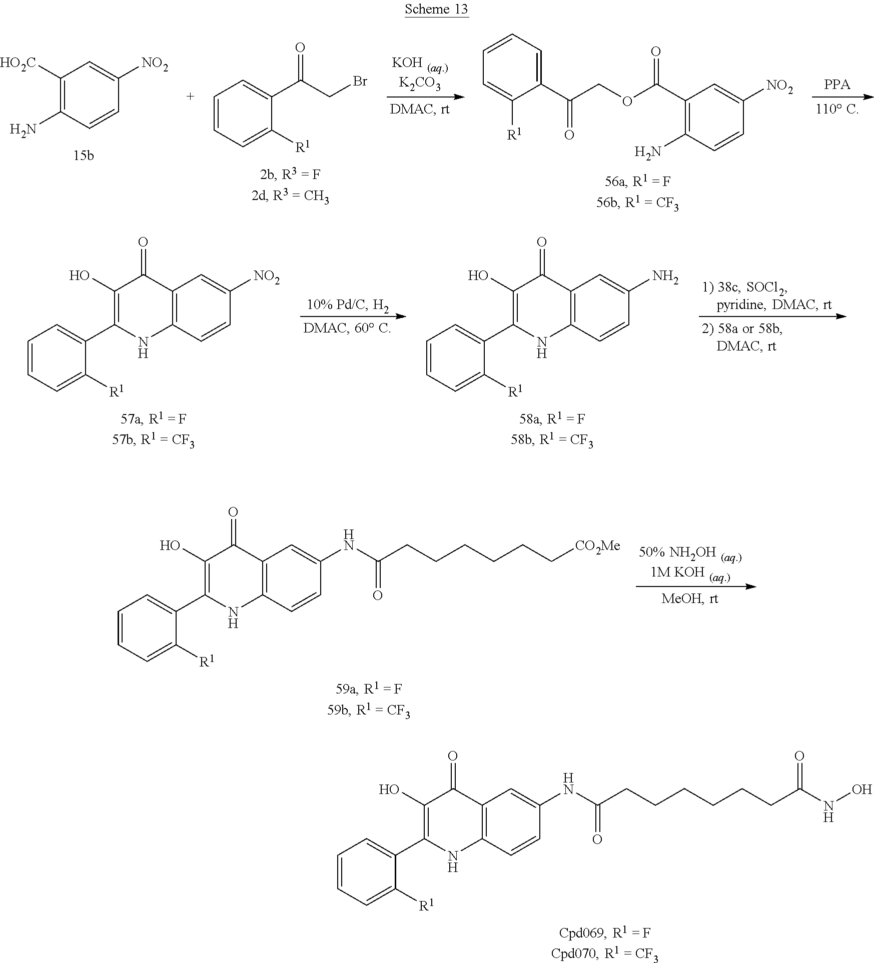

### Preparation of 2-(2-fluorophenyl)-2-oxoethyl 2-amino-5-nitrobenzoate (56a)

The procedure for 56a was identical to that described for 53a, except that 2-amino-5-nitrobenzoic acid (15b, 1.8 g, 9.7 mmol) and 2-bromo-1-(2-fluorophenyl)ethan-1-one (2b, 2.0 g, 9.2 mmol) were used as the starting materials. The desired product (56a) was obtained as a pale yellow solid (2.3 g, 78% yield). ESI-MS: 319.4 [M+H]⁺.

### Preparation of 2-oxo-2-(o-tolyl)ethyl 2-amino-5-nitrobenzoate (56b)

The procedure for 56b was identical to that described for 53a, except that 2-amino-5-nitrobenzoic acid (15b, 3.0 g, 14 mmol) and 2-bromo-1-(o-tolyl)ethan-1-one (2d, 2.7 g, 15 mmol) were used as the starting materials. The desired product (56b) was obtained as a pale yellow solid (4.0 g, 90% yield). ESI-MS: 315.5 [M+H]⁺.

### Preparation of 2-(2-fluorophenyl)-3-hydroxy-6-nitroquinolin-4(1H)-one (57a)

The procedure for 57a was identical to that described for 54a, except that 2-(2-fluorophenyl)-2-oxoethyl 2-amino-5-nitrobenzoate (56a, 2.3 g) was used as the starting material. The desired product (57a) was obtained as a yellow solid (2.2 g, 99% yield). ESI-MS: 301.6 [M+H]⁺.

### Preparation of 3-hydroxy-6-nitro-2-(o-tolyl)quinolin-4(1H)-one (57b)

The procedure for 57b was identical to that described for 54a, except that 2-oxo-2-(o-tolyl)ethyl 2-amino-5-nitrobenzoate (56b, 4.0 g) was used as the starting material. The desired product (57b) was obtained as a yellow solid (3.6 g, 95% yield). ESI-MS: 297.4 [M+H]⁺.

### Preparation of 6-amino-2-(2-fluorophenyl)-3-hydroxyquinolin-4(1H)-one (58a)

A suspension of 2-(2-fluorophenyl)-3-hydroxy-6-nitroquinolin-4(1H)-one (57a, 2.2 g, 7.3 mmol) and 10% Pd/C (460 mg, 50% wet) in 30 mL of DMAC was stirred at room temperature for 3 hours under a H₂ atmosphere. Upon completion, the reaction mixture was filtered through a Celite® pad. The filtrate was poured into water and the resulting precipitate was collected by filtration. The desired product (58a) was obtained as a brown solid (1.2 g, 61% yield). ESI-MS: 271.6 [M+H]⁺.

### Preparation of 6-amino-3-hydroxy-2-(o-tolyl)quinolin-4(1H)-one (58b)

The procedure for 58b was identical to that described for 58a, except that 3-hydroxy-6-nitro-2-(o-tolyl)quinolin-4(1H)-one (57b, 1.0 g) was used as the starting material and the reaction was stirred in DMAC at 60° C. for 3 hours. The desired product (58b) was obtained as a brown solid (828 mg, 92% yield). ESI-MS: 267.1 [M+H]⁺.

### Preparation of methyl 8-((2-(2-fluorophenyl)-3-hydroxy-4-oxo-1,4-dihydroquinolin-6-yl)amino)-8-oxooctanoate (59a)

Compound 59a was prepared following the general procedure described above using 58a (200 mg, 0.74 mmol) and 38c (209 mg, 1.11 mmol) as starting materials. The desired product (59a) was obtained as a brown solid (280 mg). ESI-MS: 441.2 [M+H]⁺.

### Preparation of methyl 8-((3-hydroxy-4-oxo-2-(o-tolyl)-1,4-dihydroquinolin-6-yl)amino)-8-oxooctanoate (59b)

Compound 59b was prepared following the general procedure described above using 58b (400 mg, 1.50 mmol) and 38c (424 mg, 2.25 mmol) as starting materials. The desired product (59b) was obtained as a brown solid (620 mg). ESI-MS: 437.1 [M+H]⁺.

### Preparation of N¹-(2-(2-fluorophenyl)-3-hydroxy-4-oxo-1,4-dihydroquinolin-6-yl)-N¹-hydroxyoctanediamide (Cpd069)

Cpd069 was prepared following the general procedure described above using 59a (280 mg) as starting material. The desired product (Cpd069) was obtained as a brown solid (226 mg, 69% yield over 2 steps). ¹H NMR (500 MHz, DMSO-d6) δ 11.75 (br s, 1H), 10.33 (br s, 1H), 10.03 (s, 1H), 8.65 (br s, 1H), 8.45 (d, J=2.2 Hz, 1H), 7.78 (dd, J=9.0, 1.9 Hz, 1H), 7.66-7.51 (m, 3H), 7.44-7.34 (m, 2H), 2.32 (t, J=7.4 Hz, 2H), 1.95 (t, J=7.4 Hz, 2H), 1.66-1.56 (m, 2H), 1.55-1.45 (m, 2H), 1.36-1.23 (m, 4H). ESI-MS: 442.3 [M+H]⁺.

### Preparation of N¹-hydroxy-N⁸-(3-hydroxy-4-oxo-2-(o-tolyl)-1,4-dihydroquinolin-6-yl)octanediamide (Cpd070)

Cpd070 was prepared following the general procedure described above using 59b (620 mg) as starting material. The desired product (Cpd070) was obtained as a beige solid (519 mg, 79% yield over 2 steps). ¹H NMR (500 MHz, DMSO-d6) δ 11.61 (br s, 1H), 10.33 (br s, 1H), 10.01 (s, 1H), 8.66 (br s, 1H), 8.44 (d, J=2.2 Hz, 1H), 8.12 (br s, 1H), 7.76 (dd, J=9.1, 2.2 Hz, 1H), 7.53 (d, J=9.0 Hz, 1H), 7.45-7.31 (m, 4H), 2.32 (t, J=7.4 Hz, 2H), 2.22 (s, 3H), 1.95 (t, J=7.4 Hz, 2H), 1.66-1.56 (m, 2H), 1.55-1.45 (m, 2H), 1.36-1.22 (m, 4H). ESI-MS: 438.3 [M+H]⁺.

### Example 14

### NOX Inhibition Assay

### Nox1 HT29 Cell-Based Assay

HT29 cells were cultured in T75 flask (Corning) until 70-80% confluence was reached. Cells were trypsinized, harvested in Hank's Balanced Salt Solution (HBSS) and counted. 1-2×10⁵ cells were seeded into individual wells in 70 μl final volume (96 well plates, Corning) and treated for 30-60 mins at 37° C. with 10 μl of test compounds and DMSO. After incubation, 20 μl of a mixture containing 100 μM chemiluminescent probe L012 plus 0.4 units of HRP (final concentration) was added. Luminescence was recorded every 5 min over a period of 60 min by using a SpectraMax i3 Multiplate reader (Molecular Devices, Sunnyvale, CA, USA). Compounds were tested in duplicate and data analysis was performed using GraphPad Prism (GraphPad, La Jolla CA, USA).

### Nox4 HEK293 Overexpression Cell-Based Assay

Stably overexpressing NOX4 HEK293 cells in log-phase growth were trypsinized, and dispersed with a density of 3×10⁴ in 100 μl of 1× Krebs-Ringer phosphate glucose (KRPG) buffer containing test compounds and incubated at 37° C. for 30 min. After incubation, cells were mixed with 100 μl of Amplex Red reagent solution (Catalog number: A22188; Invitrogen, Carlsbad, CA, USA) containing 50 μM Amplex Red and 0.1 units/ml of HRP in KRPG buffer. The samples were incubated at 37° C. for 90 mins in the dark, and then centrifuged at 2,500g for 5 mins. The supernatants were loaded to a 96-well black plate, and then measured using a SpectraMax i3 Multiplate reader (Molecular Devices, Sunnyvale, CA, USA) at 530 nm excitation and 590 nm emission respectively. Compounds were tested in duplicate and data analysis was performed using GraphPad Prism (GraphPad, La Jolla CA, USA).

### HDAC Enzyme Assays

The pan-HDAC in vitro activity is detected by HDAC-Glo™/II Assay and Screening System (#G6420, Promega, WI, USA). This system is single-reagent-addition, homogeneous, luminescent assays that measure the relative activity of histone deacetylase (HDAC) class I and II enzymes from cells, extracts or purified enzyme sources. The assay procedure is as follows:
1. Seed cells in 100 mL culture medium with 10000 cells/well in white 96-well culture plate (Corning) and incubate overnight.
2. Prepare test compounds and SAHA stock in DMSO and dilute stock to ten-fold of working concentration by HDAC-Glo™ I/II Buffer (Provided in HDAC-Glo™ I/II Assay and Screening System). Use 10% DMSO in HDAC-Glo™ I/II Buffer as non-inhibitor control.
3. Remove medium in white 96-well plate, wash cells with PBS, and add 90 mL fresh serum free medium to wells.
4. 10 mL of non-inhibitor control and ten-fold working concentration compounds were directly added to the wells and incubate for 2 hours at 37° C.
5. Add 25 mL substrate (HDAC-Glo substrate in HDAC-Glo™ I/II Buffer+0.1% Developer reagent+1% Triton X-100) to wells and the chemiluminescence signal was recorded every 5 min over a period of 60 mins in a SpectraMax i3 Multiplate reader (Molecular Devices, Sunnyvale, Calif., USA) for 1 h. The activities are expressed in percentage of non-inhibitor control minus blank control.

As set forth in Table 1-7, below, IC₅₀ values are defined as follows: IC₅₀≤0.1 μM (+++); IC₅₀>0.1 μM and 0.5 μM (++); IC₅₀>0.5 μM (+).

TABLE 1
Activity of compounds of Formula (IIIa)
(IIIa)

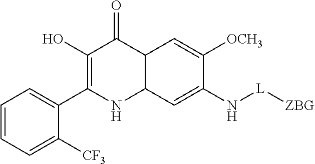

| | | | | | pan- |
|---|---|---|---|---|---|
| | | | NOX1 | NOX4 | HDAC |
| Compd | L | ZBG | IC₅₀ | IC₅₀ | IC₅₀ |
| Cpd007 | *—CH₂—* | 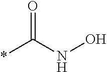 [SMILES: *C(=O)NO] | +++ | +++ | |
| Cpd009 | *—CH₂—C(O)—NH— (CH₂)₄—* | 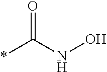 [SMILES: *C(=O)NO] | +++ | +++ | + |
| Cpd010 | *—CH₂—C(O)—NH— (CH₂)₅—* | 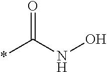 [SMILES: *C(=O)NO] | +++ | +++ | + |
| Cpd011 | *—CH₂—C(O)—NH— (CH₂)₆—* | 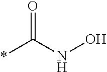 [SMILES: *C(=O)NO] | +++ | +++ | ++ |
| Cpd012 | *—CH₂—C(O)—NH— (CH₂)₇—* | 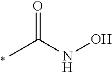 | +++ | ++ | + |
| Cpd019 | *—CH₂—* | 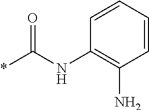 [SMILES: *C(=O)Nc1ccccc1N] | +++ | +++ | |
| Cpd020 | *—CH₂—* | 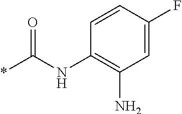 [SMILES: *C(=O)Nc1ccc(F)cc1N] | +++ | +++ | |

TABLE 2
Activity of compounds of Formula (IIIb)
(IIIb)

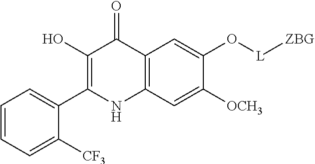

| | | | | | pan- |
|---|---|---|---|---|---|
| | | | NOX1 | NOX4 | HDAC |
| Compd | L | ZBG | IC₅₀ | IC₅₀ | IC₅₀ |
| Cpd008 | *—CH₂—* | 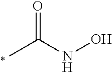 | +++ | +++ | |
| Cpd013 | *—CH₂—C(O)—NH— (CH₂)₂—* | 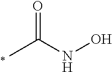 | +++ | +++ | + |
| Cpd014 | *—CH₂—C(O)—NH— (CH₂)₃—* | 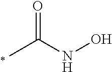 | +++ | +++ | + |
| Cpd015 | *—CH₂—C(O)—NH— (CH₂)₄—* | 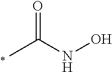 | +++ | +++ | + |
| Cpd016 | *—CH₂—C(O)—NH— (CH₂)₅—* | 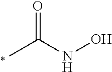 | +++ | +++ | + |
| Cpd017 | *—CH₂—C(O)—NH— (CH₂)₆—* | 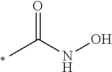 | +++ | +++ | ++ |
| Cpd018 | *—CH₂—C(O)—NH— (CH₂)₇—* | 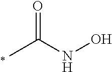 | +++ | +++ | + |
| Cpd025 | 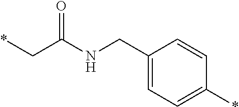 [SMILES: *CC(=O)NCc1ccc(*)cc1] | 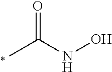 | +++ | ++ | + |
| Cpd027 | 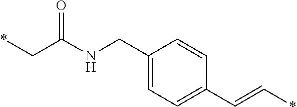 [SMILES: */C=C/c1ccc(CNC(=O)C*)cc1] | 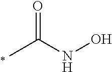 | +++ | +++ | + |
| Cpd021 | *—CH₂—* | 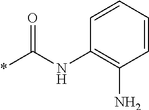 [SMILES: *C(=O)Nc1ccccc1N] | +++ | +++ | |
| Cpd022 | *—CH₂—* | 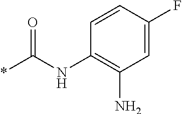 [SMILES: *C(=O)Nc1ccc(F)cc1N] | +++ | ++ | |
| Cpd023 | 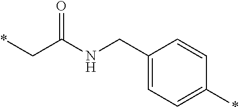 [SMILES: *CC(=O)NCc1ccc(*)cc1] | 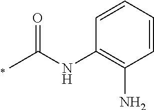 | +++ | +++ | + |
| Cpd024 | 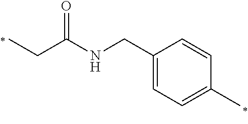 | 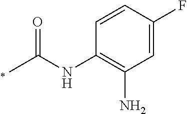 | +++ | ++ | |
| Cpd026 | 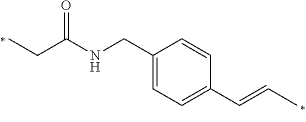 | 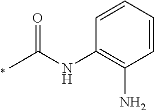 | ++ | ++ | |

TABLE 3
Activity of compounds of formula (IVa)
(IVa)

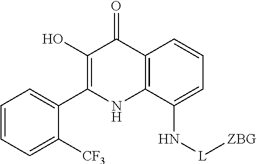

| | | | | | pan- |
|---|---|---|---|---|---|
| | | | NOX1 | NOX4 | HDAC |
| Compd | L | ZBG | IC₅₀ | IC₅₀ | IC₅₀ |
| Cpd040 | *—C(O)—(CH₂)₄—* | 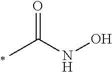 | +++ | +++ | + |
| Cpd041 | *—C(O)—(CH₂)₅—* | 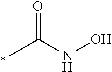 | +++ | +++ | +++ |
| Cpd042 | *—C(O)—(CH₂)₆—* | 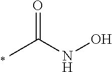 | +++ | +++ | ++ |
| Cpd043 | *—C(O)—(CH₂)₇—* | 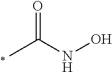 | +++ | +++ | + |
| Cpd050 | 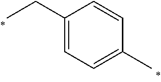 | 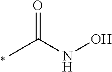 | +++ | +++ | + |
| Cpd053 | 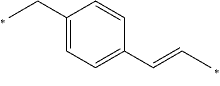 | 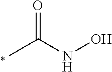 | ++ | +++ | + |
| Cpd047 | 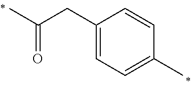 | 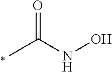 | +++ | +++ | + |
| Cpd057 | 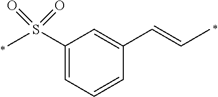 | 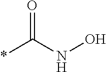 [SMILES: *C(=O)NO] | +++ | +++ | +++ |

TABLE 4
Activity of compounds of formula (IVb)
(IVb)

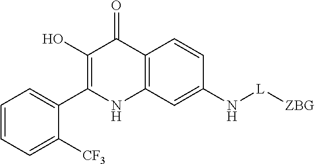

| | | | | | pan- |
|---|---|---|---|---|---|
| | | | NOX1 | NOX4 | HDAC |
| Compd | L | ZBG | IC₅₀ | IC₅₀ | IC₅₀ |
| Cpd036 | *—C(O)—(CH₂)₄—* | 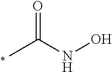 | +++ | +++ | ++ |
| Cpd037 | *—C(O)—(CH₂)₅—* | 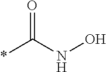 [SMILES: *C(=O)NO] | +++ | +++ | +++ |
| Cpd038 | *—C(O)—(CH₂)₆—* | 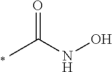 | +++ | +++ | +++ |
| Cpd039 | *—C(O)—(CH₂)₇—* | 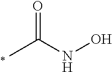 | +++ | +++ | ++ |
| Cpd049 | 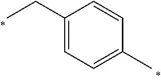 | 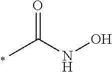 | +++ | +++ | + |
| Cpd052 | 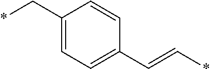 [SMILES: */C=C/c1ccc(C*)cc1] | 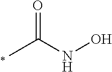 | +++ | +++ | + |
| Cpd062 | 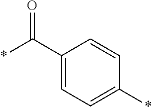 [SMILES: *C(=O)c1ccc(*)cc1] | 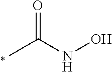 | +++ | +++ | ++ |
| Cpd046 | 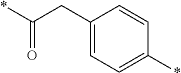 [SMILES: *C(=O)Cc1ccc(*)cc1] | 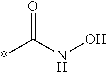 [SMILES: *C(=O)NO] | +++ | +++ | ++ |
| Cpd063 | 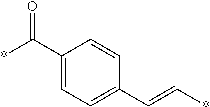 [SMILES: */C=C/c1ccc(C(*)=O)cc1] | 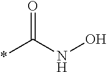 [SMILES: *C(=O)NO] | +++ | +++ | + |
| Cpd060 | 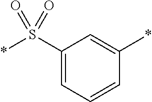 [SMILES: *c1cccc(S(*)(=O)=O)c1] | 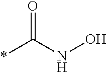 [SMILES: *C(=O)NO] | +++ | +++ | + |
| Cpd056 | 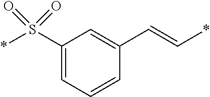 [SMILES: */C=C/c1cccc(S(*)(=O)=O)c1] | 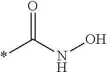 [SMILES: *C(=O)NO] | +++ | +++ | +++ |
| Cpd061 | 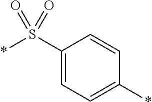 [SMILES: *c1ccc(S(*)(=O)=O)cc1] | 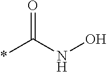 [SMILES: *C(=O)NO] | +++ | +++ | + |
| Cpd058 | 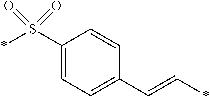 [SMILES: */C=C/c1ccc(S(*)(=O)=O)cc1] | 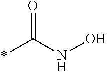 [SMILES: *C(=O)NO] | +++ | +++ | + |
| Cpd059 | 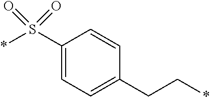 [SMILES: *CCc1ccc(S(*)(=O)=O)cc1] | 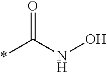 [SMILES: *C(=O)NO] | +++ | +++ | + |

TABLE 5
Activity of compounds of formula (IVc)
(IVc)

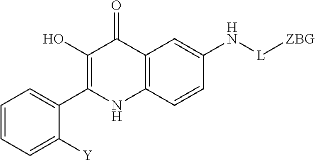

| | | | | | | pan- |
|---|---|---|---|---|---|---|
| | | | | NOX1 | NOX4 | HDAC |
| Compd | Y | L | ZBG | IC₅₀ | IC₅₀ | IC₅₀ |
| Cpd032 | CF₃ | *—C(O)—(CH₂)₄—* | 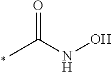 | +++ | +++ | + |
| Cpd033 | CF₃ | *—C(O)—(CH₂)₅—* | 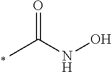 | +++ | +++ | ++ |
| Cpd034 | CF₃ | *—C(O)—(CH₂)₆—* | 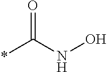 [SMILES: *C(=O)NO] | +++ | +++ | ++ |
| Cpd069 | F | *—C(O)—(CH₂)₆—* | 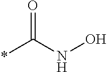 [SMILES: *C(=O)NO] | +++ | +++ | +++ |
| Cpd070 | CH₃ | *—C(O)—(CH₂)₆—* | 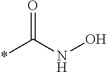 [SMILES: *C(=O)NO] | +++ | +++ | ++ |
| Cpd035 | CF₃ | *—C(O)—(CH₂)₇—* | 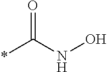 [SMILES: *C(=O)NO] | +++ | +++ | ++ |
| Cpd048 | CF₃ | 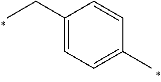 | 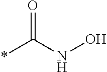 [SMILES: *C(=O)NO] | +++ | +++ | ++ |
| Cpd051 | CF₃ | 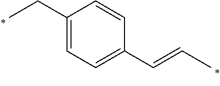 | 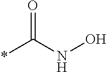 [SMILES: *C(=O)NO] | +++ | +++ | ++ |
| Cpd045 | CF₃ | 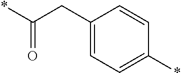 [SMILES: *C(=O)Cc1ccc(*)cc1] | 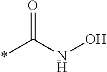 [SMILES: *C(=O)NO] | +++ | +++ | ++ |
| Cpd055 | CF₃ | 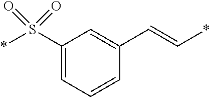 [SMILES: */C=C/c1cccc(S(*)(=O)=O)c1] | 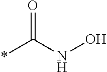 [SMILES: *C(=O)NO] | +++ | +++ | ++ |

TABLE 6
Activity of compounds of formula (IVd)
(IVd)

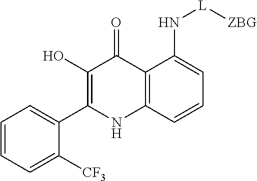

| | | | | | pan- |
|---|---|---|---|---|---|
| | | | NOX1 | NOX4 | HDAC |
| Compd | L | ZBG | IC₅₀ | IC₅₀ | IC₅₀ |
| Cpd028 | *—C(O)—(CH₂)₄—* | 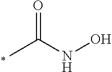 | +++ | +++ | + |
| Cpd029 | *—C(O)—(CH₂)₅—* | 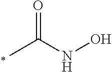 | +++ | ++ | ++ |
| Cpd030 | *—C(O)—(CH₂)₆—* | 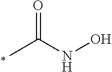 | +++ | ++ | ++ |
| Cpd031 | *—C(O)—(CH₂)₇—* | 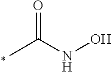 | ++ | ++ | + |
| Cpd044 | 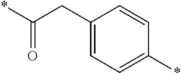 [SMILES: *C(=O)Cc1ccc(*)cc1] | 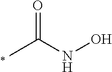 | ++ | +++ | + |
| Cpd054 | 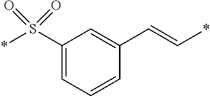 [SMILES: */C=C/c1cccc(S(*)(=O)=O)c1] | 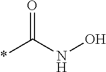 [SMILES: *C(=O)NO] | ++ | ++ | + |

TABLE 7
Table 7 Activity of compounds of formula (V)
(V)

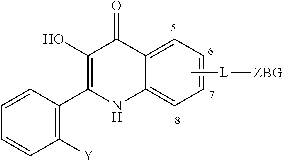

| | | | | | | pan- |
|---|---|---|---|---|---|---|
| | | | | NOX1 | NOX4 | HDAC |
| Compd | Y | L | ZBG | IC₅₀ | IC₅₀ | IC₅₀ |
| Cpd064 | CF₃ | 6*—CH═CH—* | 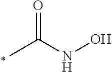 | +++ | +++ | +++ |
| Cpd066 | F | 6*—CH═CH—* | 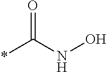 [SMILES: *C(=O)NO] | +++ | +++ | +++ |
| Cpd068 | CH3 | 6*—CH═CH—* | 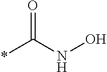 [SMILES: *C(=O)NO] | +++ | +++ | ++ |
| Cpd067 | OCH₃ | 6*—CH═CH—* | 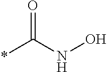 [SMILES: *C(=O)NO] | +++ | +++ | ++ |
| Cpd065 | CF₃ | 7*—CH═CH—* | 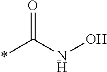 [SMILES: *C(=O)NO] | +++ | +++ | + |

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

1. A compound of the following Formula (I): 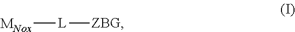
or a pharmaceutically acceptable salt thereof,
wherein,
ZBG is a hydroxamic acid group, an ortho-aminoanilide group or a derivative thereof;
M_{Nox} is or comprises a binding moiety that binds to Nox, and has a formula derived from a compound having the following Formula (II): 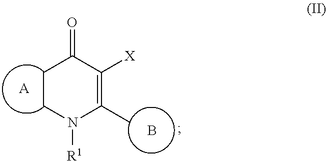
and
L is a bond or a linker covalently coupling M_{Nox} and ZBG, and is covalently linked to any location on the M_{Nox},
wherein,
A is an aminobenzene moiety, an ortho-dimethoxybenzene moiety, or a benzene moiety;
B is a phenyl group substituted with one or more nitro, methyl, CF₃, halogen, pyrrolidine, OR², NR³R⁴, or SO₂R³;
R¹ is H or C₁-C₁₀ alkyl;
R² is methyl, ethyl, n-propyl, or CF₃;
R³ is methyl, ethyl, n-propyl, or CF₃;
R⁴ is H; and
X is OH or NH₂.

2. The compound of claim 1, wherein ZBG is 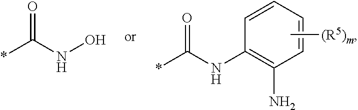
wherein each R⁵ is independently hydrogen or halogen; and
m is 1, 2, 3 or 4.

3. The compound of claim 2, wherein ZBG is

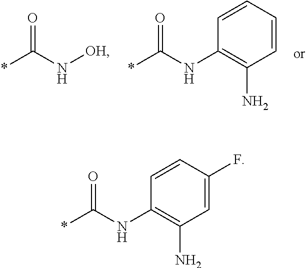

4. The compound of claim 1, wherein L is selected from the group consisting of:
*—(CH₂)ₙ—*, *—CH₂—C(O)—NH—(CH₂)ₙ—*, *—C(O)—(CH₂)ₙ—*, *—NH—C(O)—(CH₂)ₙ—*, *—CH═CH *, 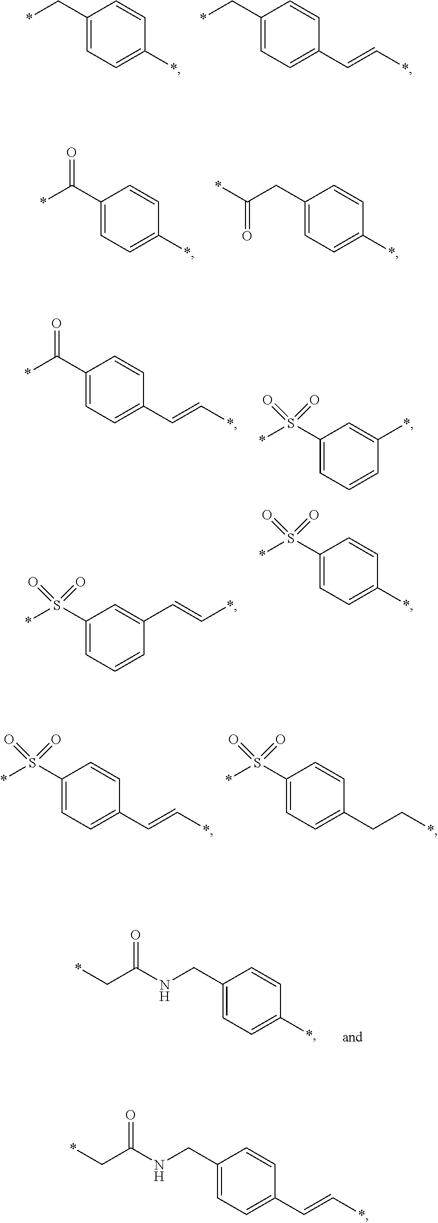
and wherein n is 1, 2, 3, 4, 5, 6, 7, 8, 9, or 10.

5. The compound of claim 1, which has the following Formula (Ia): 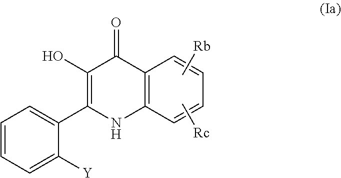
wherein,
one of Rb and Rc is *—O-L-ZBG, *—NH-L-ZBG, or *-L-ZBG;
the other of Rb and Rc is H or OCH₃; and
Y is halogen, methyl, CF₃, OCH₃, OCH₂CH₃, OCH₂CH₂CH₃ or OCF₃, or a pharmaceutically acceptable salt thereof.

6. The compound of claim 5, which has the following Formula (III): 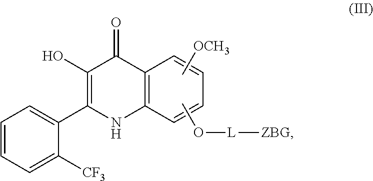
or a pharmaceutically acceptable salt thereof.

7. The compound of claim 6, which has the following Formula (IIIa): 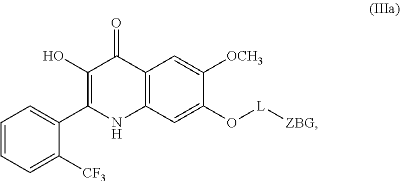
or a pharmaceutically acceptable salt thereof.

8. The compound of claim 6, which has the following Formula (IIIb): 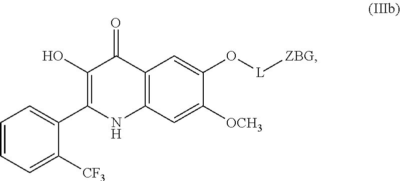
or a pharmaceutically acceptable salt thereof.

9. The compound of claim 5, which has the following Formula (IV): 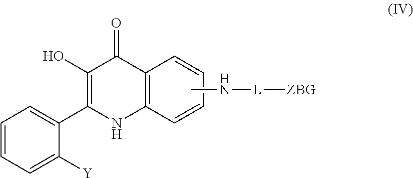
wherein Y is halogen, methyl, CF₃, OCH₃, OCH₂CH₃, OCH₂CH₂CH₃ or OCF₃,
or a pharmaceutically acceptable salt thereof.

10. The compound of claim 9, wherein Y is CF₃, F, CH₃, or OCH₃.

11. The compound of claim 9, which has the following Formula (IVa): 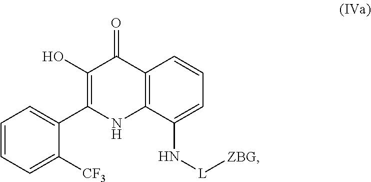
or a pharmaceutically acceptable salt thereof.

12. The compound of claim 9, which has the following Formula (IVb): 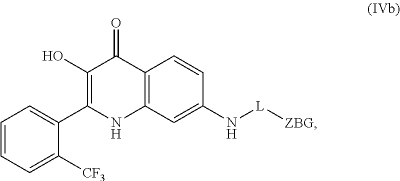
or a pharmaceutically acceptable salt thereof.

13. The compound of claim 9, which has the following Formula (IVc): 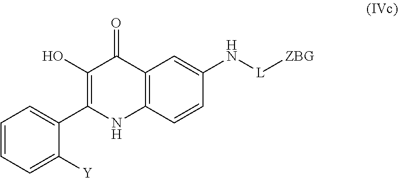
wherein Y is CF₃, F, CH₃, or OCH₃,
or a pharmaceutically acceptable salt thereof.

14. The compound of claim 9, which has the following Formula (IVd): 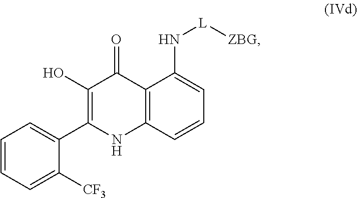
or a pharmaceutically acceptable salt thereof.

15. The compound of claim 5, which has the following Formula (V): 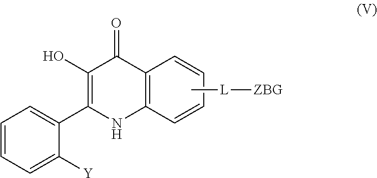
wherein Y is halogen, methyl, CF₃, OCH₃, OCH₂CH₃, OCH₂CH₂CH₃ or OCF₃,
or a pharmaceutically acceptable salt thereof.

16. The compound of claim 15, wherein Y is CF₃, F, CH₃, or OCH₃.

17. The compound of claim 1, which is any one selected from the group consisting of:
N-hydroxy-2-((3-hydroxy-6-methoxy-4-oxo-2-(2-(trifluoromethyl)phenyl)-1,4-dihydroquinolin-7-yl)oxy)acetamide,
N-hydroxy-5-(2-((3-hydroxy-6-methoxy-4-oxo-2-(2-(trifluoromethyl)phenyl)-1,4-dihydroquinolin-7-yl)oxy)acetamido)pentanamide,
N-hydroxy-6-(2-((3-hydroxy-6-methoxy-4-oxo-2-(2-(trifluoromethyl)phenyl)-1,4-dihydroquinolin-7-yl)oxy)acetamido)hexanamide,
N-hydroxy-7-(2-((3-hydroxy-6-methoxy-4-oxo-2-(2-(trifluoromethyl)phenyl)-1,4-dihydroquinolin-7-yl)oxy)acetamido)heptanamide,
N-hydroxy-8-(2-((3-hydroxy-6-methoxy-4-oxo-2-(2-(trifluoromethyl)phenyl)-1,4-dihydroquinolin-7-yl)oxy)acetamido)octanamide,
N-(2-aminophenyl)-2-((3-hydroxy-6-methoxy-4-oxo-2-(2-(trifluoromethyl)phenyl)-1,4-dihydroquinolin-7-yl)oxy)acetamide,
N-(2-amino-4-fluorophenyl)-2-((3-hydroxy-6-methoxy-4-oxo-2-(2-(trifluoromethyl)phenyl)-1,4-dihydroquinolin-7-yl)oxy)acetamide,
N-hydroxy-2-((3-hydroxy-7-methoxy-4-oxo-2-(2-(trifluoromethyl)phenyl)-1,4-dihydroquinolin-6-yl)oxy)acetamide,
N-hydroxy-3-(2-((3-hydroxy-7-methoxy-4-oxo-2-(2-(trifluoromethyl)phenyl)-1,4-dihydroquinolin-6-yl)oxy)acetamido)propenamide,
N-hydroxy-4-(2-((3-hydroxy-7-methoxy-4-oxo-2-(2-(trifluoromethyl)phenyl)-1,4-dihydroquinolin-6-yl)oxy)acetamido)butanamide,
N-hydroxy-4-(2-((3-hydroxy-7-methoxy-4-oxo-2-(2-(trifluoromethyl)phenyl)-1,4-dihydroquinolin-6-yl)oxy)acetamido)pentanamide,
N-hydroxy-4-(2-((3-hydroxy-7-methoxy-4-oxo-2-(2-(trifluoromethyl)phenyl)-1,4-dihydroquinolin-6-yl)oxy)acetamido)hexanamide,
N-hydroxy-4-(2-((3-hydroxy-7-methoxy-4-oxo-2-(2-(trifluoromethyl)phenyl)-1,4-dihydroquinolin-6-yl)oxy)acetamido)heptanamide,
N-hydroxy-4-(2-((3-hydroxy-7-methoxy-4-oxo-2-(2-(trifluoromethyl)phenyl)-1,4-dihydroquinolin-6-yl)oxy)acetamido)octanamide,
N-(2-aminophenyl)-2-((3-hydroxy-7-methoxy-4-oxo-2-(2-(trifluoromethyl)phenyl)-1,4-dihydroquinolin-6-yl)oxy)acetamide,
N-(2-amino-4-fluorophenyl)-2-((3-hydroxy-7-methoxy-4-oxo-2-(2-(trifluoromethyl)phenyl)-1,4-dihydroquinolin-6-yl)oxy)acetamide,
N-(2-aminophenyl)-4-((2-((3-hydroxy-7-methoxy-4-oxo-2-(2-(trifluoromethyl)phenyl)-1,4-dihydroquinolin-6-yl)oxy)acetamido)methyl)benzamide,
N-(2-amino-4-fluorophenyl)-4-((2-((3-hydroxy-7-methoxy-4-oxo-2-(2-(trifluoromethyl)phenyl)-1,4-dihydroquinolin-6-yl)oxy)acetamido)methyl)benzamide,
N-hydroxy-4-((2-((3-hydroxy-7-methoxy-4-oxo-2-(2-(trifluoromethyl)phenyl)-1,4-dihydroquinolin-6-yl)oxy)acetamido)methyl)benzamide,
(E)-N-(2-aminophenyl)-3-(4-((2-((3-hydroxy-7-methoxy-4-oxo-2-(2-(trifluoromethyl)phenyl)-1,4-dihydroquinolin-6-yl)oxy)acetamido)methyl)phenyl)acrylamide,
(E)-N-hydroxy-3-(4-((2-((3-hydroxy-7-methoxy-4-oxo-2-(2-(trifluoromethyl)phenyl)-1,4-dihydroquinolin-6-yl)oxy)acetamido)methyl)phenyl)acrylamide,
N¹-hydroxy-N⁶-(3-hydroxy-4-oxo-2-(2-(trifluoromethyl)phenyl)-1,4-dihydroquinolin-8-yl)adipamide,
N¹-hydroxy-N⁷-(3-hydroxy-4-oxo-2-(2-(trifluoromethyl)phenyl)-1,4-dihydroquinolin-8-yl)heptanediamide,
N¹-hydroxy-N⁸-(3-hydroxy-4-oxo-2-(2-(trifluoromethyl)phenyl)-1,4-dihydroquinolin-8-yl)octanediamide,
N¹-hydroxy-N⁹-(3-hydroxy-4-oxo-2-(2-(trifluoromethyl)phenyl)-1,4-dihydroquinolin-8-yl)nonanediamide,
N-hydroxy-4-(2-((3-hydroxy-4-oxo-2-(2-(trifluoromethyl)phenyl)-1,4-dihydroquinolin-8-yl)amino)-2-oxoethyl)benzamide,
N-hydroxy-4-(((3-hydroxy-4-oxo-2-(2-(trifluoromethyl)phenyl)-1,4-dihydroquinolin-8-yl)amino)methyl)benzamide,
(E)-N-hydroxy-3-(4-(((3-hydroxy-4-oxo-2-(2-(trifluoromethyl)phenyl)-1,4-dihydroquinolin-8-yl)amino)methyl)phenyl)acrylamide,
(E)-N-hydroxy-3-(3-(N-(3-hydroxy-4-oxo-2-(2-(trifluoromethyl)phenyl)-1,4-dihydroquinolin-8-yl)sulfamoyl)phenyl)acrylamide,
N¹-hydroxy-N⁶-(3-hydroxy-4-oxo-2-(2-(trifluoromethyl)phenyl)-1,4-dihydroquinolin-7-yl)adipamide,
N¹-hydroxy-N⁷-(3-hydroxy-4-oxo-2-(2-(trifluoromethyl)phenyl)-1,4-dihydroquinolin-7-yl)heptanediamide,
N¹-hydroxy-N⁸-(3-hydroxy-4-oxo-2-(2-(trifluoromethyl)phenyl)-1,4-dihydroquinolin-7-yl)octanediamide,
N¹-hydroxy-N⁹-(3-hydroxy-4-oxo-2-(2-(trifluoromethyl)phenyl)-1,4-dihydroquinolin-7-yl)nonanediamide,
N-hydroxy-4-(2-((3-hydroxy-4-oxo-2-(2-(trifluoromethyl)phenyl)-1,4-dihydroquinolin-7-yl)amino)-2-oxoethyl)benzamide,
N-hydroxy-4-(((3-hydroxy-4-oxo-2-(2-(trifluoromethyl)phenyl)-1,4-dihydroquinolin-7-yl)amino)methyl)benzamide,
(E)-N-hydroxy-3-(4-(((3-hydroxy-4-oxo-2-(2-(trifluoromethyl)phenyl)-1,4-dihydroquinolin-7-yl)amino)methyl)phenyl)acrylamide,
(E)-N-hydroxy-3-(3-(N-(3-hydroxy-4-oxo-2-(2-(trifluoromethyl)phenyl)-1,4-dihydroquinolin-7-yl)sulfamoyl)phenyl)acrylamide,
(E)-N-hydroxy-3-(4-(N-(3-hydroxy-4-oxo-2-(2-(trifluoromethyl)phenyl)-1,4-dihydroquinolin-7-yl)sulfamoyl)phenyl)acrylamide,
N-hydroxy-3-(4-(N-(3-hydroxy-4-oxo-2-(2-(trifluoromethyl)phenyl)-1,4-dihydroquinolin-7-yl)sulfamoyl)phenyl)propanamide,
N-hydroxy-3-(N-(3-hydroxy-4-oxo-2-(2-(trifluoromethyl)phenyl)-1,4-dihydroquinolin-7-yl)sulfamoyl)benzamide,
N-hydroxy-4-(N-(3-hydroxy-4-oxo-2-(2-(trifluoromethyl)phenyl)-1,4-dihydroquinolin-7-yl)sulfamoyl)benzamide,
N¹-hydroxy-N⁴-(3-hydroxy-4-oxo-2-(2-(trifluoromethyl)phenyl)-1,4-dihydroquinolin-7-yl)terephthalamide,
(E)-N-(3-hydroxy-4-oxo-2-(2-(trifluoromethyl)phenyl)-1,4-dihydroquinolin-7-yl)-4-(3-(hydroxyamino)-3-oxoprop-1-en-1-yl)benzamide,
N¹-hydroxy-N⁶-(3-hydroxy-4-oxo-2-(2-(trifluoromethyl)phenyl)-1,4-dihydroquinolin-6-yl)adipamide,
N¹-hydroxy-N⁷-(3-hydroxy-4-oxo-2-(2-(trifluoromethyl)phenyl)-1,4-dihydroquinolin-6-yl)heptanediamide,
N¹-hydroxy-N⁸-(3-hydroxy-4-oxo-2-(2-(trifluoromethyl)phenyl)-1,4-dihydroquinolin-6-yl)octanediamide,
N¹-hydroxy-N⁹-(3-hydroxy-4-oxo-2-(2-(trifluoromethyl)phenyl)-1,4-dihydroquinolin-6-yl)nonanediamide,
N-hydroxy-4-(2-((3-hydroxy-4-oxo-2-(2-(trifluoromethyl)phenyl)-1,4-dihydroquinolin-6-yl)amino)-2-oxoethyl)benzamide,
N-hydroxy-4-(((3-hydroxy-4-oxo-2-(2-(trifluoromethyl)phenyl)-1,4-dihydroquinolin-6-yl)amino)methyl)benzamide,
(E)-N-hydroxy-3-(4-(((3-hydroxy-4-oxo-2-(2-(trifluoromethyl)phenyl)-1,4-dihydroquinolin-6-yl)amino)methyl)phenyl)acrylamide,
(E)-N-hydroxy-3-(3-(N-(3-hydroxy-4-oxo-2-(2-(trifluoromethyl)phenyl)-1,4-dihydroquinolin-6-yl)sulfamoyl)phenyl)acrylamide,
N¹-(2-(2-fluorophenyl)-3-hydroxy-4-oxo-1,4-dihydroquinolin-6-yl)-N⁸-hydroxyoctanediamide,
N¹-hydroxy-N⁸-(3-hydroxy-4-oxo-2-(o-tolyl)-1,4-dihydroquinolin-6-yl)octanediamide,
N¹-hydroxy-N⁶-(3-hydroxy-4-oxo-2-(2-(trifluoromethyl)phenyl)-1,4-dihydroquinolin-5-yl)adipamide,
N¹-hydroxy-N⁷-(3-hydroxy-4-oxo-2-(2-(trifluoromethyl)phenyl)-1,4-dihydroquinolin-5-yl)heptanediamide,
N¹-hydroxy-N⁸-(3-hydroxy-4-oxo-2-(2-(trifluoromethyl)phenyl)-1,4-dihydroquinolin-5-yl)octanediamide,
N¹-hydroxy-N⁹-(3-hydroxy-4-oxo-2-(2-(trifluoromethyl)phenyl)-1,4-dihydroquinolin-5-yl)nonanediamide,
N-hydroxy-4-(2-((3-hydroxy-4-oxo-2-(2-(trifluoromethyl)phenyl)-1,4-dihydroquinolin-5-yl)amino)-2-oxoethyl)benzamide,
(E)-N-hydroxy-3-(3-(N-(3-hydroxy-4-oxo-2-(2-(trifluoromethyl)phenyl)-1,4-dihydroquinolin-5-yl)sulfamoyl)phenyl)acrylamide,
(E)-N-hydroxy-3-(3-hydroxy-4-oxo-2-(2-(trifluoromethyl)phenyl)-1,4-dihydroquinolin-6-yl)acrylamide,
(E)-N-hydroxy-3-(3-hydroxy-4-oxo-2-(2-(trifluoromethyl)phenyl)-1,4-dihydroquinolin-7-yl)acrylamide,
(E)-3-(2-(2-fluorophenyl)-3-hydroxy-4-oxo-1,4-dihydroquinolin-6-yl)-N-hydroxyacrylamide,
(E)-N-hydroxy-3-(3-hydroxy-2-(2-methoxyphenyl)-4-oxo-1,4-dihydroquinolin-6-yl)acrylamide, and
(E)-N-hydroxy-3-(3-hydroxy-4-oxo-2-(o-tolyl)-1,4-dihydroquinolin-6-yl)acrylamide,
or a pharmaceutically acceptable salt thereof.

18. A compound of the following Formula (IIb): 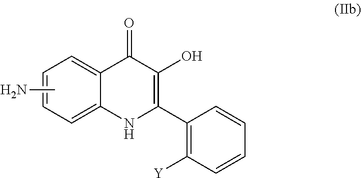
wherein Y is halogen, methyl, CF₃, OCH₃, OCH₂CH₃, OCH₂CH₂CH₃ or OCF₃,
or a pharmaceutically acceptable salt thereof.

19. The compound of claim 18, wherein Y is CF₃, F, CH₃, or OCH₃.

20. The compound of claim 18, which is any one selected from the group consisting of:
5-amino-3-hydroxy-2-(2-(trifluoromethyl)phenyl)quinolin-4(1H)-one,
6-amino-3-hydroxy-2-(2-(trifluoromethyl)phenyl)quinolin-4(1H)-one,
7-amino-3-hydroxy-2-(2-(trifluoromethyl)phenyl)quinolin-4(1H)-one,
8-amino-3-hydroxy-2-(2-(trifluoromethyl)phenyl)quinolin-4(1H)-one,
6-amino-2-(2-fluorophenyl)-3-hydroxyquinolin-4(1H)-one, and
6-amino-3-hydroxy-2-(o-tolyl)quinolin-4(1H)-one,
or a pharmaceutically acceptable salt thereof.
